SEQUENCE LISTING (9 sequences)

SEQ ID NO: 1 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHINQLQQIMRLTGTPPAYLINRMPSHEARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 2 - DNA, 95330 bp, Homo sapiens
agttattgttctcttctcgcgagaactgctcctcccatcctcttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtggtgagtgtcgctgggcctggggccgctgtgggcagggtggcccctcgcgcccgagggccaggcctgctccactgctcagcgttgcgtcaagtggcaggaattttcctcgggggagggcattgctgccccttcgagctctgcccgttctgcacctccagcacccctcgccctgcactcacgcaggtatgcggtccaccgtgtgcagcactcaggtccgctgcgagaggtaggtggtggtgctggagctcggttctggctagcaccctgcgccttcccctctcggagggttgctgctcggcaacaggcaccgggggaagggccgcttccttgggggtctccctgcctacctggagcagaaggaccctctcccgtgatgcgcccacgctggggctccggaccctgggtcctctgagcagacaagctcggggaactgccgggagaagcagaagggcacagcccaacccgaagactgcggtcatctgaacaaaactgaccaggaagggagctctctccgggcctttttttttttttttttttttcaaaactctgtcgaaatcccatcttgaaaagcgttctttttgaattcgcacttgaattaacagcgatccatagagcttaaaataccgactttatctcggtgagcactgtgccagcttgagtggtgtgtttccgattcggttgaaagcgtatttcctttccgtggaggggcggcggtggtagtgcctgcaagaacactgcagcttgagaaactggcaggctcactgattagtcacataccactgctcatttaagtattgttcgacaaaacaattttctcatttggagcaagaaagattcagatctgtaaggcgccctcaagatcaaaaagttattaacagtgctgggaggtattgtaaatattttttgagataattcctgcgaatactgtgttttaaaagtcccttagagagcttgcagggagttatctagttatctgcatttatggcctgaggtacacgatggttttcgtaaggatatggaaccacatagaaaaggaagggaagagatactttttagctttaatttacaccgtgagtgtgttttaaattaagtcgatagcatataatttccagacctgaaatatttcactaactgctctcttaacttccaaggttttataccctgtattttagattttctataatttgaaagctacggatcttaaactaagtatcagatttaactgttagctttttctgtgttttagtaattcgtattgtatgcgtcatttgaaaacgggcctgtaaaatcgtttttttgttttggaacaaattccaggcttttggatgtgttaatctttaaatatgatttctgtgtttcccaaagtcacatcatgaatttcctgtttggtagaatcttctgggtaattgagattgtgcaattgtgtccagcaacagatcatattgcattttctagttcaagttgcttaagttatgttgcagatagcaccgtgcttgacatctttctcccatttttacagcatggcagtttacattttaaaggaatttgcagcattttacattcagagagtgtgtgtgtgtagttggtttgcagcattttacattcagtatatacatattatataggtatatacattcagtatatacatattatataggtatatacattcagtatatacatattatatatatctagttgatttgctaattttcaaagacgtatttggatcattgctcttttaatattttatagctcattaatatacttccattttttcattcttgaattgacatacatttctgtagtatcacgttttcgttttcaatgctatttaaattttaatggagtgatacagcagaataactcaactatagagggtttgggtgaactccaattgaattgcttgcaagggttcgctactattggtgactagctctgcactggaacaggagattcagatttacagtgaagtaccattaaaattaactccacagttctttagtggtatacttttctgtggtatagaagccagcagtatctgacacttaaaagtacttttattttattgaacagattttttaaaatacattgacattgtataaaatttgaaagttttacaaaggtactcagtgggccgggtgcagtggctcacgcctgtaatcccagcactttgggaggccgaggcgggtggatcacagcgtcaggagatcgagaccatcctggctaacacggtgaaaccccgtctctactaaaaatacaaaaaattagccgcccgtggtggcgggcgcctgtactcccagctactcaggaggctgaggcaggaggatgatttgaacccgggaggcggaggttgcaatgagctgagatcgctccactgcactccagcctgggcgaaagtgcgagacgccgtctcaaaaaaaaaaaaaaaaaagatattcagtgaaaagtctcctttcctttttgttctccagccactctactctctcaaagacaggcactgttaagtgtcttgtgtctttccagagaggcagttttttttatttgcttgtttgtttagtagtctttctactaaagggatttaagatgtaacctacagcatattggtgaaagaaaatacaactgaaactgaaattgtatgacaattgacaataaacaaattataactaatgttggaatgaaaataccagaatttagatttacaattaagtgttatcttattcagagattcagcgtagttatttgaagtttttcttttctttgagacgaagtctcactctgtcgcccaggcttgagtgcagtggtgccatcttggctcactgcaacttccacctcctgggttcaagcgattctcctgcctcagcctccctagtagctgggactacaggtgcccgccaccatacccagctaatttttgtatttttagtagagacggggttctccatgttggccaggctggtctcagactcctgacctcaagtgatctgctctccttggcctcccaaagtgctgggattataggcctcagccacggtgcccagcctgaagttgttctttcaatgagtacctttttaggtgtggcagaaaacattttttggaacttcttttagaatccactttggagcctgtgatattcttcttcttttttattttgagacagagtctcattctgttgtccaagttggagtgcagtggcaccatctctgctcactataacctccacctcatgggttcaagcgattctcctgccttaggctcccaagtagctgggatacaagtgcgcaacaccatgcccggatgatttttgtatttttaatacagacagggtttcaccatgttggccaggctgatctctaactcctgacctcaggtgatccacttgccttggcctcccaaagtgctgagattacaggggtgagccaccgtctctggccatccatcttcatttttttgagagcagatttgattttagggaataacagttcatttgaatccaaataatttagggaatttggactcaagtctgataagaaagataaggcaatcaggttggtgatactactgttttaggtcagataggagttataactataaagtaacaacacataattttttgtgtgacttcttgaactggacctgaagtctacttccaaaaaaatctccgaattttattttatttatttatttattttgtagagatagggtcttactgtgttgcccaggttggtctcaaactcctggcctcaagcagttcccccaccttggcctcccgaagtgctgggattatagggatgagccaccacacccagcccctgaattttattttcaagaatgaaaataaatgaatagaatatttcaatgaataaaattcattcattgaaatatttgtcagtttccgaattttattgctctaaagtgtggtatactttgtaacatttacaaattattttaagcctatattattttaatttatactaagtatagtaaaaaaacaagtagaagcctgtgtgataaccaactatgaatcaaaatagagttttacaaatatgcctttccgttaaagggtaaagggaactagactaggtcttgcagttttaaggaactttatgcattgtctgtgaggggaaatgaagcaaaattgtttaagaatacattgttgcaaagcattgattttggggttttgggacagaaggggagaatctttgtggtgagtttaaggaaatacaaacaggaacttaagtgcctttcctctggggtagggttcttccaggcccttgctgttgattagaggaggtgggacactggtaaccatgaaataggaggtgacagttgagaattactcttagatcttattcaactgtttattcaacagtgtttattgaatacctaccatgggccagatagctaggttctggaaatagagtgctaaacaagacagaaacaagacaatccattctctcctggaacttacagcctagtgggtttaataactacattttgtggattgcttgctatgttctagcattgtgctgtgcactttatcatcagcataaaaagtatataacaaaacctttggtaaaaggatagcaaaacttgctaagaaatcaaatacaacttcctaagtgctttgtggaacaagattattctagtataaaagaaataagtgaagctaactttttttaactattaatggccattcaccattttaaatctatttttttttttttttagaatgagccagggcacaaagaaataaaagtgcatgattattaagtatacaagggtgactttcttgactcctgaccagtgagaagggtgtgccctttacttttctagtagtgatagtaggtccatgctgagctttccattcttttctttgtaaccaaaagtaaggtagttgcctgtatgttaccagtgccagtcaataagaagtctttaaacataattaacggcaagcatttgtattattgatgtataaagattgttttaagaaagagttcattcgaagctggctataaagtcattttattatcaaatcccatttttccacttttttttttttttgagacggagtcttgctgtgttgcccaggctggggcgcagtggtgcaatctcggctcacggcagcctctgcctcccgagttcaagcaattctcctgcctcagcctcctgaatagctgagattacaagtgcgtgccaccatgcctggctgatttttgtatttttagtagagacggggttttaccatgttggtcaggccagtctcgaactcctgaccttgtgatctgcccaccttggcctcccaaagtgctgggattacaggaatgagccaccacacccagcctccactggttcttctttaaaataagcttatcctggatctccaggttttcagttctgccagtttgatttcttgatactgaggagtactctgcttttgatttctttctagcccagtctcagagatgatgtgttgactttgcctttcatgggcaagagtgcagatattgctgagcccctggggcctagaattcaaagatggaagaattcactggggtagatcaccctcactttagaattaactgacatttttagggatttttgcagtttataaagtgttttcattcaaagcattatcaatcagttctcactagctccatggaggctcatatttattaactccattttatagatggggagctggactcattcattaaaggaatggcatacttgtcaatgctacttttagtagtctcctttctgatagatgtgggctgctgaggttgcgcggtggcaccttctcttatgcagttaacctcctcttgtgaaaaaggggttgctgagtcactcggaatagagcttgagtgtaggaaagcagttccaccagtgtcattgcccagtttatgtgaggggtaatgacttaacccatttaacccattagagtcatgtctcttcctcagaactgataaatcaaaaatgcactcattgcccagattaaaattttttctgaaggaattattaggcagtgcaaatagttaattcctgaatttatgaatcctattggattgaaattcttcagtgtacatttatgtaaagttgggactgtctgtacatgtttgtgttacataagtataggatataaatagaaataactgcctctccagccctttgaaaatacattagatttttgctttttgcagtaaaatttggggtaaaactgatttgttcttatctaagaggtataattactgcagtgctcagaaaagcctcattataaagtgggctcccctgacttggaacttagggatgtcttctcatggctgctatggaagtcatgcagctgtttcgtcaggaaaataaccaaaaacttctttaaacttggaaatgtttttgtaaaagaaactttcagtattcagagtagtttgtctttcacagtgcttccataaattaagcacaggcacaactcgttttattatgcttcacagataattgctttttttttgcaaattgaaggttagtgacaaccctgtggtaagcgagtcttttggcaccatttttccaaaagcatgtgctcacttcatgtctctgtgtcacattttggtaattctcgcaatatttctttctttctttttttttttttttttttgagacggagttctgctctgtcgcccagactggagtgccatggtgcgatctcggctcactgcaacctccgccttccggtttcaagtgattctcctgcctcagcctcccaagtagctgggactacaggcatgtgccaccacacctggctaatttttgtatttttagtagagatggggtttcaccatgttggtcaggctggtctcgaactcctgaccttgtgatctgcctgccttggcctcccaaagtgctgggattacaggtgtgagccaccatgcccggcagttctgctgggattacaggtgtgagccactgtgcccggcagttctcgcaatatttcaaactttctcattattattaaatattatggggatctgagatcactgatctttgatgttgctgttgtaattgttttgaggtgtctggaaccatgcccataggcagacttaattgataaacattgtgtgtgttctgactgctctgccaaccagctgttccctctctccatctcctaaggcgtccttattccctgagacacaataatactgaaattaggccaattaataacactacagtggtttctgaatgttcaagtgaaaggaatgtttgcatgtctcttactttaaatcaaaagctagaaatgattaagcatagtgaggaaggcatgtcgaaaactgagataagctgaaagctaggcttcttgtaccaaacagccaatttgtgaatgcaaatgaaaagtttttgaaggaaattaaaaattatattccagtaaacaaatgaatgattagaaagcaacacagccttgttgctgagctggagaaagttttagtggtctggataaaagatcagaccaaccacaacatgcccttaagtcaaagcctaatctagagcaaagccctaactctctttaattgtatgaagtttgagacaggtgagaaagctgcaaaagataagtttgaagctagcagagggtggttcataggtttaaggaaagaacctgtctccatagcataacttatagtgcaaggtggctgggggcggtggctaacacctgtaatttcagcactttgggaggctgaggcaggcagatcatctgaggtcaggagtttgagaccagcctggccaacatggcgaaactctgcctctagaaaaaatacaaaaatttgttttttatattttttggtgttgtggtgtacacctatagtcccagctgcttgggaggctgaggcaggagaattgcttgaacctgggaagtggaggttgcagtgagccactgcaaccaagtggcaaccgagatcgtgccactgcactccagcctgggcgatagagcaagagtacatctcaaaaaaaaaaaaaaaaaatcaatagatgagaaattgcttctcaggaatgaataaataaagtagtttcttgaggtggaatctgctgttggggaagatgcttatgaacattgttgaaatgacaacaaagggtttagaatattacataaacttagttgataaagcagcagcaaggtttgagaggattgactcccattttgaaagtagttctactgtgggtaaaatgctatcaaacaccattgcaggctacagagaaatctatcacgaaaggaagaataaatcaatgcagcaattcaccgttgtcttatttttagaaattgccatagtcattccagtcttcagcagttatcaccctgagtagtcagcagccatcaccctgagtaggcagcagccatcaacactgagacaagaccctccaccagcaagaagataataacttgctgaaggctcaggtgacccttagcattttttagcaaaaaaatatttatttatttatttattttttctagatggagtctcgctctgttgcccagcctggagtgcagtggcacgatcttggctcactgcaccctccacctcccaggttcaagcaattctcccaccttagcctcccgagtagctgggattacaggtgtgcaccaccatgcccagccaatttttgtatttttaatagagatggggtttcaccatgttggctaggctggtctcaaactcttgacctcaagcgatcctcctgcctcggcctcccaaagtgctgagagtaataggtatgagccactgtgcccggctaataaaatatttttaaattaaggtatgtgcatatatatatttttttgacatactgctagtgcatacttaatagactacagtatcatgtaaatataacttttatatgcactgggaaaccaaaaaatttatgtcactcatttttttgtgatattcactgtgttgtggttgtctggaactgaactcatgatatctttgaggtacacctgtatgtatgtgttaaggatttacaaaattaaccagctttcccgtgcagtgttcaagtcttttaataatttattatagggaagaagaagtggtggtttgtaaattctgtacttgacaaatggatactgggttttgggcatgcttggctccaaggctgaatgttgggggaaagaagtataggagagtgaggagtctaggatttctaagttttggccttgggcaaatggatatattttagtgccattttctgaactttgtgttacaaagttacaaaagagtgttacagggtacaagattgtagagagaaaggggaaggagcataggttcatggtaatctccttgaggacaggactcattataccttgagtgcctaccatgtatggtttgccgtaagtgctggagatgaaatgtgaacaaaagagacaaaaatttctgccttaagaagctatattctggccaggtgcagtgagtggctcacacctgtaattcctgcactttgggaggccaaggtaggtttccttgagcccagtagttagaaaccagcatcctgggaaaaatggggaggctcctctactaaaaaataaaaataaaaaattagggatgcttggtggcacactcctgtagttccaactacttgggaggctgaggtggagggattgcttgagcctaagaggattgcttgagcctgggaagttgaggctgtagtgagctgtgatcctgccactgtactccagcttgggtgacagagtgataccctgtctccaaaaagaaaaaaaaaaggaagctatattctaatggggatggcagtggaagtagataataaacaaaatatatagtgtgtcagataagtgctgtggagaaaatgcaagaagggaaggagagtgagtgctaggatggctgttgcacttttaagaaaggtgaatacggtaagcatcattgataaagtaacaattgagcaaaaacctgaagaaggtgatataggacactggttggacatgggtgaggatgtgataacattctaggctgaggattagcaagtgtgcaaaagccctgaggtaagggccctgtgctccaggaaatagctaagagaccagtacagctagagctaaatgaaccaggggccaaatatagggagaaaagtggggtgaggagagggttgcagatatgcagggatcatgtagggtcttatttgcctttgtacagactgtgacttttcctttaagtgagaggggaagccttggagggttatgaccagaggaaggacatgacctaaatcttgttttaacagattactgtgctctagggtcccaagtttttcgtttcggttccttacccattctaccctaaaccactactgtgaataggatagaacggtggagttagatgaattggtgcaagccctgacccatgctcacaactctcattccagcagtcccagagtgccagagccagtatttttgttgggccctagaaatttagggtattccagtccagctctttaaataacatgggctgactttactttttcaggtttttgttgccaaaaggaaggagcagagattagctcttgagtttagctgaaaaaggaagattagtctgctggcagattgagtgttttttaacttctttggcttttagggtttttcttacttttggatcatgttttaaaagtcatttttaccttctttcaacagttaaacatttaagttttatttgattttatttgaccctttttctgctttacattcctctgccttttccccattataatacttttagtagaggttagaccattgttatctgtcattaatgtcttagctttgaggccccaaggacttctctgttaaacttaaatatatcatatagattgaaagattatctacagacatgtgatcctataaaaacccatattatttattgtcctccctgcctttacagatgtaaacccctctcctcttttcatcagagctccccctcttttactgctctatcatcaggttgcagaaaataagttttcctgcaaacatagattcccatggcaacataacaaaaaggtaatagaacaaatgtttattttgtctagacctgctgtctgagtaccattagaccctgaacatgttggctcctgttatttgaatatgtggttacaaattccttattttcctcaaaacttacttctcttaggccttggtgatattgtcactgtgcctgtgatatgaaggaaggtgtgtgtgtgtgtgagtatgtgtgtcttactgtcattctactcaaaagtgtgtattgattgcattttcaaacttatggcttaaaatgtagacaccacacaatctgagcatgtgaagaaaaaaagcataccagttttgttctttgttttcgtaagttcattaatttaatatttccagtgcactatactagatattttgtttacagagattttaaaaaaaagaagggtccctcctttcgaaagtatcagtccaggctgggtgcagtggctcacgcctgtaatcccagcactttgggaggccaaggtgggcggatcacttgaggtcaggagtttgagatcagcctggccaacatggtgaaaccccatctctacaaaaattagttgggcatggtggtgtgtgcctgtaatcccagctattcaggaggctgaggcacaagaatcacttgaacccgggaggcggaggttgcagtgagccgagatcactcccctgcactcctgtctgggcgatagagcaagactcagtctcaaaaaaaaaaaaaaaaagaaagtatcagtccagaggagacatatttatgtttcctcatagatggctttggaagtcctctgtagttttggatcttgaagcattttaggttttggattttcagattagggatactcaacctgtataacccagggtaattcaacctattgacactttaggtgggataatgttttgttgtgggggactgacctgtgcagtgtagtatgtttagcagcatccctgtctccaactcactgtatactaatagcaattcccagttgtgacaaccaaaaatgtctagacattgccacatatctcttgacgggtagggagggcaaaatcacctttagttgagaaccacttatataaccctatatatttggattgataaccctgtatatttgattgagtaaaaactagatgggataacatgaaccttccacctgcaaaaaggcagaaatgctatactttctgcattataaattgggcctctctaaacatgatttttgttatttctaggagagtaacagcatttctaatggtcaaggtcctagctataacccaataagcttaatggatgggagaacttgcacaagcatgtttgcaagaggccaggtgtatcactggcacatttgagaaacaactaattagtgagggagacccaattttcatgaataattgtgttttgtcattgtcatggccccaacttagggtgttttgaattttttctggaaaggaaattagcatgaagtccaagttggtaggtaggtagcattttccctagtgtctcttctggtattctgtgtcctttctctcctgtcttctttcagtatttagcgttttcaaactcctagaaataaaatgctgtttagaagaccacaaagagagctgcacataaactaaggagcaagaagtagtacattgtgaattaattttctgtatgtttttgaaggttcttgcatctgggaaggcttgcttagttttaaaaatattaggtaataacagactgaaattttgaagatcttgtggatctcaggtagcagtaactcaggaagatcagccttctttgatttagaatgtcatttaggcacattatgtattctggtaccttagaagatacaatgcattttcctgaattatcattgctactgctttaattactttaagggtgtgaggcagtaactaactttagtgtcacaatgtatacatcttaacatttattgtgctcttttttttttttttaaacaccaccttaaagtactctgctattcagatttttccttgtgtttctctaattctcctatttcatccattggtcagttttttcttcctcttttctttctgctccatgatccattcttaaattttcttttctctttctgtgcatattctttttgaattcattatctgatagtctcaacaatggtcttgttcatatactatccttccaaatttgtactttcaaacctaatctcaagaatgactgatcctttggattctaaatctattgagatgtaatgtttcatgcttttccttgattactttcctacacacttcttgaagataccagagatgtagcactaacttccgttggaatgggattcatgttacaagcttgaatggttcagtttagatatgtgaagcggatgtcatcattagcactgatgactgacagtagttataaaaattacaatcaggaatgaaccaatttacctgaagttttcaataactctaggtattaatgcaacataattttgaaagtttgaaatatataggtataaatattgatgcttatattttagatttactgtagtgaaggaaatttgccagtcgtccatcagtgttaaatgcccaccaatgtgtaaaagaaatagacctaaaatattaataaattgtgtatatatgtatgaaggtattgatgtgtacagagaagtggagacattgtgagttatagtggtcagtatggttttatggagcagaaaggatttgaagattatttagaaagatgggtatttggagagaagaaaaggaaacattttcagacaagagaaaacataaaccaagagaatagatatgaaaaggactgttctgacaggaggaacatctcaggtagggtgaacccagttgtggttctcattcttgttgtgcttcagactcacttgtggaacttttaaacatacctctggaaattaagaatgagatctggagtgggacttaggcagttgcatttctaaaaagtctacaaatggttctttaggtagtagagagctgttggagtttcttgagttgtgaactgtggaggaaaattgttttaaaattggtcttggtggagtgtaaagaatggattagaggaaacaaatgtattgctttgtcaaagtctggtaaatttatgtgtgcctctgttcattttgactcctcggctagatttcttagttccttgaggtcataaacaatcagcttttaaagcttcagtgaaggaatatctttttttttttttttttttttggctgggttgggggacagggttttgctctgtcacctgggtggaagtgtagttacgcagtcaatggctcactgcagccttgaactcctgagttcaagtgatcttcctacctcagccacccaaagtagctgagactacaggtgtgtgccgccatgcccacctaattttttttttttttcgtagacacgaggtctcactatgtcatgaagttgcccaggctggtcttgaactcctgagctcaagcttcctcctgcctcagacttccgaagtgctaggattacaggtgtgagccaccgcacctagcaggaaacatcttataaaatcattctactttcatttggaacttgcaggctcgtgcctgcaatcccagcactttggcaggctgaggcaggtgaattacttgagctcaggagctcaggatttctaaaccagcctgggcaacatggtgaaactttgtctctaccaaaaatataaaaaaattagccaggcgtcgtggtgtgcctcagtagtagcagctactcgggaagctgaggtgggaggtttgttggggcctgggaagtcaaagctgcattgagccgtgatcatgccaccacactctagcctgggcaacagagcaagaccccgtctcaaaaaaaaaaattaaaaatgaaagtaagtttcattaaaaaatactatatatgtaattacagtggtgaaattgttttgtgattaaataaatactgttgtgttgacattaaaacaagatatttgagagttagcgaagatattaaagctaatgttttaaattttgagtttttaaagtcaatcttaaatctcctgctactctgaagaagataaatgtatataattttaatttttctatttcactaggtttgaagaatattttttgaggttcctaaatcaaaacttaagttccaatttttgcataaagttgcctttattttcaagacgttatttccaaaatggcaggacagagcatccgcttcagagcaacagtgatatttgcacatttacttgaatatggaaggaagaaaagctaggcttctgtaatagaatgtaacatgtgcagaaaaatatttccagtatgtcatttattgaaatttgttccaggtgctacaggtaaagagaaagttccaagttagtggtcttaattatttagttatttagaaggcaagttttgaagactattattaattatcctgattcagttggaaaaaaagcctgaaattgtatgatccggagaagaggtgtcatgtttttgcaaattatttcttctcactttatagatattcttttttctttttctttctttttttttttttttttttttttgagatggagtttcgctcttgttgcccaggctggagtgcaatggcacaatctcagctcaccacaacctctacctcccgggttcaagcgattctcctgtgtcagcctcccaagtagctgagattataggcatgcaccaccacacctggctaattttgtatttttagtagagacgggatttctccatgttggtcaggctggtcgcgaatacccaacctcaggtgatctgcccacctcggcctcccaaagttctgggattacagtcgtgagccaccgcacccggccctcactttatagatattctaatgaatttaatgtattaatgagactttgctaattcagtgtaactacagatttcaatatatttaaatatttccattaacttccaaaagtaaaccaaattgtgtttatcttcttagaagtcatctatgaccattgaaagaaattgtaaaatcacttacgatccccacaaattactcaaagacttgctgagtgattactcattcttcatattctgttgatgaaaacacagattgttttttgaggaaaatcatggtgcagtcgtttgtactttaatactctgtggacatgggcacttgtccagcaccttacctttcttaagaggttgtctccctttctctactcagctgatgttaaatttattgatctgccattaccttataacagaaaatctacaagtcaggtgcccatcatggaatataaacacaagttccctatatctctatacctatcaaaagctcagttgacagccgaacatggtggctcatgcctgtaacccaaacactttgagaggctgaggtgggaggatcgcctgaggccaggagttcgagaccagcctgggcaacaagcaaggccccatctctacaaaagaaaattttaaaaataataaacaaaaagctcattgatagctccagtcatctttggaggattgtctgaagaatatggagcttatctgtttttctaaatgtatttaagatttcctcttggattaagtctgtcagagaggccaggtatggtggctcatgcctgtaacccctgtacttattgggagtccaaggcaggaggatcacttagcctaggagttcaagaccagcctagggaacatagtgggaccctgtgtctacaaaaatcaaaataattagcgaagcttggtggcacatgcctgtagtcctggctacccaggaggctaaggttggaggattgcttgaggccaggaggttgaggctgcagtgagccatgattacatgactccactccagcctggatgacagagtgagaccttaaagaaaaaaaaaaaaagcaacaccgtaatattttgtactcctaccctctttgctaataatgaactgtattttggccttacgacagcaatctgtactgatggatgctctaaaaagaaaaccatctgaaacccactattatattcttttaaagatgttgattaggacatttttaccacattatatgtctccttcttgaacagacacagaatgaacttttccctggtttattacaggaataattatgcctctcttcagctctcctgagtacctggttggtttttattgtatggcttctgtgattcatgtcctaactgattctgttctctttgttaatggctttagtgagctttgagccaaatttgaggcctacctctaatgggtatttaaaaaggacctctggccgggtgtggtggctcacgcctgtaatcccagccctttgggaggctgaggtgggcggatcacgagatcaggagatcgagaccatcctggctaacacggtgaaaccccatctctactaaaaatacaaaaaaactagctgggtgtggtggcatgcacctgtagtcccagctgctggggaggctgaggcaggagaatggcgtgaacccgggaggcggtgcttgcagtgagccgagatcgcgccactgcactccagcctgggtgacacagcaagactctgtctcaaaaaaaaaaaaaaaaaaaaaaaaagaaagaaaggacctcatgatatatgctaacctcagctgtacagactttcttattttcccttaatcacttaccattttgtcaggttttatatatataattccagtctgctttaaatccattgtgaataaaggaaggtatacatcaataaatttagaagttaaacagaattatattaggatatatgaagaatctagtaacccacattttctatgtgtgtgtgacacaaggggaggcaagtatgaatgtgtaacaaaacattgaagtttatttaatgattatccttgtgtgttatgtactggattcacggtcagcagtagatgagtgagaatgacatcctaatattgtaataactgcttctaatttttatatttctttcagttattttttactataccaatgattttataaaatgtgaattaatttgataattttggaaaagttcatgtaaattcacaattgggggaaaaacttgttaaacagtatgtcctgaattttagtttggagaacctgtttatcataaagataatttcattttatgatgttgttcatgaaaacaaggttttacttgtagctctacttttacagatgtgattgagaggcgtatttgagctttgccatggaccaagctccttgccaaacctactataaaagttattacatttaatcctaacatatccaacagatgaaatataaacataattctcatttcatgaaagaatttaattaacatcctcaagattatatagctattaagtggcagaactggaattaaaatccagatagtctaattccaaagtcccagctcttatttaatcactctgttggagtattataaacatgtaataataaattgtgattgttggggtctatagggtactgattaattctagcctgccttttgctgaatagattcaggagtcaaatagagataaaaatatttacttttattagtaaagctggcatgcagagtgccttgggcgggcaactcaagtgggcttcttatttcttccccagttaattgaacttgcacacttaatcagtcagatcagctagaaaactgctgctgttccttgctccccagcaggcttaccaatggaggagagaagaaagtgttcagcttgcaggtggaaggcaaactctgaggtgggaagttgaagaagctgggtcccacaggggcaatggccatttttccagtctcaccagagctagtttagagaactgggtttagagatgagtatacatttacttgctacttttgagagggaagaagttactaatttatgggtttgtaggagcaatgggagtggaaaagaagagaaccaccaccctcttttatgtgagataaggaaggagtcagtgagggtgttgtgacagagtaatggcttcttagaggtccctaacaggttcctgagcaggtgactatttaaaccaatacttgaaggataaggatttagctatggggtgggatgggggcggaggagaagaaaatcctaggaaccaggagcagcctgtctgaatagttaaaagaactgaaaggagctagcagaacatagcagctgagagagagggaaagagtagcacaggaacaggtggaataatcagtggtgaccagatcacctgggccttgtaggccatcatgagaaatttgcattttactctgtatgccaaggagattccttgaatggtttgaaggaggggagtgttgtgatctaatttgtacttttattgctattttttatttttcatttttgagacagtctcactgtatcttccaggctagagtgcagtggtgccatcatggcgcaccacaacaaggttgagcagcttcagccttcctgggctcaagcaatcttcctgcctcagcctcccaagtagctgggcatggtggcccatgcctgactaattttttttttttttttgagatgaagttttgctcttgttgcccaggctggagtgcaatggcatgatctcagctcactgcaacctccacctccctggttcaagcaattctcctgcctcagcctcccgagtagctgggattacaggtgcctgccaccacacccagctaatttttttaaatatatatttttagtagagatggggtttcaccatgttggccaggctggtctcaaactcctaaccttagcctcccaaagtgctgggattataggcgtgagccactgcgcctggctgcctaatttttaatttatttattattatttttttgagacagagtctcactctgttgctaaggcttgagtgcagtgatgcaatctcggctcagtgcaacctccacctcctggattcaagtgatcctcccacctcagactctcaagtagctggggttacaagtgtgtgccaccacaccccactaatttttatatttttttagtagagacgggtttcaccatgttggccagactggtattgaactcctgacctcaagtgatccacccgccttggcctcacaaagtgctgggattacaggcgtgagccactgcacccaacccgttttttattttttgcagagacagggtcttgctatgttgctcaggctgatctcaaacttctgggctcaagcaatcctcctgccctggcctcccaaagtgctaggattacaggcttgagccattgtgcctggcataatttatatttttaaaagactgtattggatgcatttggaaagcatattagaaggaggtaggccagtgacatggactaatatgatattggtgaaatgaagagcagttgactttgagacgtcttttggaggtgaaaaccatgagacttgccaatagatttgaatgaggactgaggaaagggagaaatcaaggatgacaatcaaatgtctagctcgagcaactggatgaacagtccatttattgagataacagagccatggagaagaatagttctgggtgttttgaacttggtacgcttcaggtgcttatgtgtctttcttatggagacatcagggctcagagaagagatttcagtgagagacataaatttgggagttttcaatatatagatggcatttaatttaaagtcatgggcatagatgagattatctagaaagagaatgtgggagagagaagagggcccagtaccaagtcctgaagaactccaacatacagagtttgagtagaagcagcacatgcagtggagacagagatggagctgccagaggggtcatagaaaaccaatggaagtgttgatatagactccaggatggacagtatttagagaaagtggtgactatgtcaattactaatcttagaaaagtggatttggtagcagaggtcattaatgaccttgagaagagcagtttcagtggaattggattgaggaaagaatgggattgaaatgagtcagttgtgtagatgtgtgcttctcacaccttaagaggtatattaatcacctggaaatcttgtcaaaaagtaaattctattttagggcccaagatctcctttcctttccttttcctttcttttcttttttcctttcctgttgtctgcagagatcctgcattttctaatgagctcccagataatgtcaatgctgccggtccatggagcacactttgaatagcaaaggtataaattagtggttctcaaactgtaatatgtattagaataacatggagagcttgataaaatacagattattagacccacttcaaagtttcttattccattaactctggggtggtacctgagaattttcatttcttataaattcccaggtgaaactgatgctgctgatctgggaacctctggtgtaggtgacttttttggagaattttaactgtgatggagaactgttaagggaagtcttttgtttgttggctgaaggcggtaaacaagtagagcaaatttatgctgatgagaatggtccagtagatagggagacactgatgatgcaagagagaagaggaatgaagaagtgaaatccttgagaagatgagggaggtggctccaaagcatgtattgtggtcttgaattttgataagcaggggcatattcttagttatagcaagagggaaggtggagacgatgggtttatagatttggggctaagcaaatggagctcccctcctgatggtttctgttttctcagtgaaggcaagggcaactcttgagggaagagggtgaattggagatttgagagagaacatgtcaaacgatcattttcgagtaggaaaatgaacttactagaagaaaatggctttattgagagcctctttgagattaaggtcagaaactaagaatgaaaatctgattctacagtctttcttgttcaacctcagttccttggattcagtcatggagaattaagtggttgagattacccaaggttagactttatccaggtgagtacatcagagagacagacagacagagagagagaagtgcaagggagtgattataatcatggaacaggaagggagatgaagacagaagagggctgaaggatagaaacaagcaatgacaatgaggttaacaaattgttgtagtcagggtacaagtaaactagaggtaactaggtttttgatgaaaagcaaatgctgctggcattcaaaattttgtaggtggggcagtttttggtaggaataaaggtctagaatgtgaccatgaagatggctgggtgtggtggagtttcctagagaggtcaggaaacaggatgttgggtgtgccgttcacctggatgttgaagttgccaagaatgatgacagaagttgagagtgtagagagatcttctggatagaaactaacttttcaatgaatgatggaaaatttcaggtcagtggatacctgcagcagagagggatagtttatcagggttgtgagcaggaggtattttatagaacaaacggaaagtaatggtcctgaagtggtaacggctgccaaggaagactcctacgcacctgctagccaggaggtatctgggctattagagaatgctttctaatggcagggctataagagaagcagtgtcttctagaaatggcctagtttcagagatggaggggaggttcagaaaagaggttgaagatttataggagtttatcacaaattgggatttgaagattgagggtatgccataaaagtttgggagtgaaaggaagggcactggagtaggtacagagttgtaaaagcaagtttggcaggcagagagtttggtgataacagatgaccagaaagtcttggatacatgttagagactgagacaaactggaggatgaggcataatggaactggcccagacaatttctgttattactatttttttttttttttgagaccgagtttcactcttattgccaggctggagtgcgatggtgcaatctcagctcactgcaacctccgcctcctgggatcaagtgattctcctgcctcagcctcctgagtagctgggattacaggcacctgccactgcgcctaattttgtatttttagtagagatggggtttcaccatcttggtcagactggtctcgagctcctgacctcgtgatcctcccgcctcggcctcccaaggtgctggattacaggcatgagccactgtgcctggcgacgatttctaaaaataattaaaatacacatcgcatttttgctatagatgtcctccatgatctagctgctgcctgcatctcttacgtcatcccttcccactctccccttgctccctgtgctctacccacacagcctttctgtctttctgtcactcactccagcatgccatacgtttcctacctctgtttcttttttcgtttgtttgcacttgctcttctgagagatagcagagtcaagtggtcagtagcataagctctggatccagactccctgctctgccgcttttaagctttgtgaccgtggtcaagttacctaacttctctctgcttcagtttcttacttgtaagtgggagaatgataatagtacttatttcataggggattcggggaaatacaaaagctgaattgagacaaataaatgtaaagggcttagaacaatttcttgtatgtgtgggtaaagactctataaacattaatattattattatttcttctgcttgaagctctttttcttatatatttatatagggaatttaatcattatattgtttccttctcagctcagatgtcattcctcagccatcctagctaaagtggtataactccactggctttctgtcactctttatcccattacccttttatttagtcatagtattttcatcactacttgaaattattttattattctgttctgctcttcccccattccctgaaggtgccaagtactttgtaggtgctcaagaaatatttgtggcaatttattgtcctgaaaacatggtttcctgggcctcgactctgagcaggcagcagatgtggcaaggtgacgacagtgaggcctaggcagggaagacagaattcaactctgtttattaatctgttggctcatctagatcaggcagctgccagtttttgtaaattttattagaacacagccacacccattttaaaacttattttctggctgctttcctcatacaaccacagagttgagtagttgtgacagaggtcttacttacaaagttgaaaatattactatctggcccttcattgaaaaaaatttgctgacttctgatttagatacatagaaaagacaaggagaaaacaccaaaaataataacagaagctttatttggataatgggataataggtcattttgctttttatctttatgcttttttttggtattttgttatagaccctttaatttggaaatagccttataaataccccaaaataatatttagaacagctttttaatggtgggttttttcccttttttctccttagtgctgcttttgacacaaaaacggggttacgtgtggcagtgaagaagctctccagaccatttcagtccatcattcatgcgaaaagaacctacagagaactgcggttacttaaacatatgaaacatgaaaatgtaagttattcattcaaggaagaatacattttgatcttgaatagactggggaaaaatgttttaattactgcagatggaaatacgctggagtgcatcaaacgttggtccctgctccttgtcctggggtgttagtggccacttggttatgtcaggtcaaagacttggaatgaaagtgatgcatcttcctggagttttagttttactcaaagatttttagaggagaaagaactctttaaaattttaaaattaaaggatatgttatgaaatacaacacaaaattcacaataatttttaagagaaaatattaaaaagaagttatgtattctttgcctggttttcctaaggctatattatgagaatatgggagtctgtggctattaaaagacaaaatagccacagactcccatattctcataatatagccttaatatttttaagtggaaaaatctgagaaagagtgccttaggctcactgaaagtcctatcagagatggctattattttatatatttgttaataatttttctttttttccagattcacgtattttaataaatatgtcagaggcattgtttaagttaaatataactttttcttttttaccaatttgttgattcagttgccattctgcaaagatgcttacaaattctaatacgataaataagtggatataattggaatactaactgaaagtaaaaagacagttgactgaagacacttattttactgaatttcaaaatcaagttatgaatttctgtgactgctatatgaagggtactgtgaagtttgtagcattgtggaaacaatggccatagagatttcaaagtcctctggctgtactgcttgttaccttgtggaagaaaattcaaaatctgtgaatttattatttgactatttagactgttttagtaatttaacttcttcatagcgagaaaaaagtcacagataagtgactcttcattagcgtacatttgtttcattagtgcaaatttcttctaaattgagaacattaagacttaaatttgtaattggattatggatgccattataatcacagctgctgctttttattttcacattttattttgattgtatgctattgaggaaatcctatttctacagacaacttctattcctgagtataattttacttaagtagctatgtgcaaaccagagttttaagaatgttcagtgtgaagctgcagccttcctaatcagtgacatgtttacaagaagttgagatatgcggaaaaagctggcagcagaaactttattcctaggtctgcataaaatcaaagtggcctggcagctttaagggatgtgtttgcgtttgatttttaacacatttaagtgtgtatgctgtttttcattacacattattatatgtgacagagacagtgtcacagctttgcaagtataaaggatattttgaaagtgaaatgcaagccaaacatttatggaaccccagagcacctttgggagccactaggatcctattgatgtggctatgctaatttaagaacatctggtttaggagattatcagtgctacactgattgttttgcttttcttcataaatatgttaagccaatgtttagggatcatggtattagaaaaataggattttaatttctatacattgttttataagtggcttgcttactagcagtcttttttttatataaagatgtttaagttctaacagttggtggtcacctagatatcagtgatttgttatttaaacaaaataaggcacatggctgaatgtctcatagttctctattccattggcaaatgtttgaagccaaaagacttgactcaatgtagaaaaattcctgcattttagcctattttgaaaacatttgttaggtgcttataatgtcaaagtagttttacccaataaagaaccaaaacttccggtaagtttaattgagggttttcagagaaattcatctcgtttaccctcctcttcttttttaaaaaattactgttttgggtcaccatccaatttttatagcggccaaacctcccacaggcagcagcaacaggcgcttaaaacttgtgaacattttaggtgttggtaacaaatacaattttcaggtgccagagggcacatgtgatcttttacacaaaaatgcctctgacccacaatcggtagttcatcttgaattgtctcttctgttagtgatcatgaataatagataaacatttattaggtgagtgactgaagctaggggatgtcctctgtgactgtgagagtttctctctgaacaaagttagttggaccattcctgattgaacttctgaccttgatctcgttaacacctgctctaaggaaagattaaagggaggcagcctgccagctgcatcctttgctgtctgggcagacactacagagatggcccatatatgggcctagagatctgtacattttagaatttaaaaatgatcaaggaaattgttttgtaaattttatttgggagccttagatttaaccttaccattgtagaccaagagattgttttggtgtgcttttgttaaatatgctaaatattgtctctggatactgagttaaagtatttttttttggatgtgtgctaatttaataaaaatacgacttaatatttgccaggctttggaccatgatgggataatcattagagatatttgctgcttatttgtattatgaatgtcttttctaaaaatttgattcctgggttactttgaggtgactgaagtcatgtaacagcagaaggagttaaggaaatagactaaaatatgacttgatttagaagggaatttccatttaagaaatgagaaatggctgggcgcagtggctcatgcctgtaatcctagcactttgggaggccaaggtgggtggattgcttgaacccagaagtttgagaccagcctgggcaacatggcaaaaaccagtctctattattatattaaaaaaaaaaaaaagaaatgagaaatgaatagattcctaaaatattttccagatctattttaatgctgataagcttttgttttacagtattttccttatgattaatattcataagtatgattaatggagtgagggatattttgtaaaaacccatcaaaaatttaagtactttgatttagcatgggtagttttaatctcaaaattgttattttcggtgtgcttatgttaaacacattaaatattgtaaatattgccctttttaggaaattaaaatttggtagggtatttactagttggcttttctctattttttttcaggttactttatttttcttcttgcagtcatggtttttaatcatttgaatagttgagctttatgtctcatattctgttaatggaagtagctgcttcagatagtttgcttaggtatattgtattttgtaattgtctattcaactgtgtgggctgtggtcaccacttattttgatgatccacagtgggtcagccttttgtactaggcaccttcagatacattgtctgctcagtcttgctgtcagtactgtgaaaataagcaatgatgtcctcatgtttttcatatgaggaaaatgggactcagagaagataccaaatgtatttgccaattattatttacataacagccattcattgaactcctgctgaatgccaggctccattccaagtgccagcgttattggaatgaagaaattataagctcttggacccaagaaatcacaaactgatactggggttcaaacttacagctgcttggttccaaaatgtctgttgtgttttcagtttgaaattggagtttttatttgaaaatagttttgacttttgtcttttgaggtaactcttgcatgtgtaaatcagatctctaatgatttagttttatttccataggaggattttcacaatcacaatggaaacactgtaaaacaatgtaaatagttttaaacttttctattgttttgaggagatgggcatctcagataacaagaatcattgattagaaaatattggaacggtgaataaagttagcttctagacctcagttaccttaagtagaagtgggtataagggagagtgattaaaagaccagaggtcttaagccaggagtataccaaactcaagggaaacgattatggtttagatggagaaggtaaaaggaaatggatttgacttaaatttaaagcactattgtgaatgaggtcacaggtactctattgggaggcatgtaagttggccttacctctatggaggaccatttgacaatattaagatccttaaatatattggtgtcctttgattctgtaattctgtttcttgaaatttatttcctggaagaaaatatgagatgtgcaaaatacatatatttttcttgcagtattccttaaatgtctagagataggaaattggttaagttatggtatacacaaatgatagatatgctgtatacaaatatttaaatattaagtaaaaaggcaggatataaaactatgcatggtgtaaccccaatttaatttatgtacatcagaaaatcaaaggaaatatatctcaaaatgttaatgatagttataatgggtggtataattatgggtaattttggttttcttatgtactgttgtctgggtaagatctgaaagtgattctatttcctttggaacaattttcaaggacttgaaacctattcttgaattccaagtatgtgtcccaccacctccttttcaggttcagactgtaaaattaaagttgcttttaaaaagtgacatttggagtcgggcgtggtggtgcatgcctgttatcccagctactcgggaggctgaggcaggagaatcacttatacccaggaggcagaggttgcagtgagctgagattgtgccatgcactccagcctgggcaacaggagcgaaactctgtctcaaagaaaaaaaaaaaagtctgatatttggatgagtggagtaacatcctaattagaagatctttttcatgataatgttttgagtgttaacatatcttacttctagagctttgtgtttaagttggcagtttagaacagtgtgctagtgaggccacagtcttgcttcataaccagatactctcaaatatggctgggcgtattacaaatgaattatatatttcttatttgccttgtcaatgcctaactttgtagaaatgaatggcctgggttttaaggaaaatagatgggaggtaggggttggagggtgctgtatgtggataaatcagtataaattcctgatcagaaaaactgctcaaagaccatgggttattgagtattaagtagctctttgctttatccctgtgtaaaaatagactttttttttcatttttaaattgtctgatttaaataccctatacattaagactttgtgagaataataccaagaatatactgagaatattttgagactttaaaaatgtcagttttgaagcatatacagttaatatggaaatttttcgtggaatacatttttaaaaaacacaatattacggtaacaaccatggatgtgtgacctttttcacctgttggattaattcattagaccattagtacatacgtaaggcaaggaccctaaatctttttgccttctgataaactactgactacatatgaataaattctaaagagatgaagatttttaatggtaaagtgggttaatatgagaattgatgttaacaagtagaaaacaaaagcataatttgggtagaaaattagttggagttattaagtatatgatgtgatgttatggaactgaggtagctggttttgaattagttcctattaaaatgattgagacgtggctggccgtggtggctcactgctgtaatgccagcactttgggatgctgaagtgggaggatcgcttgaggccagaaggtcaagaccagcctgggcaatctagtgagacacatctgtacaaaaaaagaaaaattagctaggcatggtggtggcacgtgcttatagtcccagctacttgggaggctgagacaggattgcttgagctgcagtgaactgtgatggtgtcactgtactctagcttgtgtgtcaagagtgagatcctgactcttttttttttttttttgagacagagtctcactctgcacctgcactctcccaggctggagtgcagtggcgcaatctcgggtcactgcaacctccgcttcccaggttcaatcagttctcctgcctcagcctcccgagtagctgggattacaggcgcccgccaccacacctggctaatgtttgtatttttagtagagacagggttttaccatgttggccaggctggtctcgaactcctgacttcaggtgatccgcccgcctcggcctcccaaagtgttgggattacaggcgtgagccactgctcccggcctagaccctgactctttaaaaaaaaagaagattaagacctgatgggtactatactgagtttaatatttatttactttttaatttttatattttcttacaggtgattggtctgttggacgtttttacacctgcaaggtctctggaggaattcaatgatgtgtgagtaaattttttgcatttgccttcctggtctacagaatgaagactaatagcccatttctattcctgaaccatttagcaacatatagacttcaaaaaattctttattcctatcatgcacctctttttgggtgtagggatggataccagaagatgtatgttatgggtggtgcgtgttagtatttacatcatttcaggcacattgtaccccattgaagagtcagtcttgaattcttacaaaagttccatggagaaaggagtggagagagttttgactatggattttcttttcaaagtgtttatcccaggtttggttttgttgttgttgttgttgttgttgtttttgttttaaaagacacggggtcttgtgatattgcccaggctggactcaagctcctgggctgtagtgatcctcccacctcagcctcctgaggagctggaactacaggcacatgctaccatgcctggccgatcccaacttttaaaggtgctcttcaagaaatgtaacggatccccagccatatagactccctcctcttttatcgcccatgcatgtgggagatgatactagaactacatgctgatccttgaagtattagatttctatactgggataaggggcagactctacctgttgagaaaggcttcttctctcagctttgtaaccacctctgttgttttagcctgggatacccctagaaagcagagcctgggattctcaggaaccagtagtggggggctagggaaaatgaaacaggaaaggagagaaagcttctatgagtatatgttgagttggtcaccactatggacaattgggctgaaaactgccaagatctaagaagctgtacgtaatgcacctcagatggggatcattaatacactggctcctgaccccattggcaagggttgccacgtatgtcattagtgccttcacatgcccaggttgcacattggtgggccataagacagattccagcaagtgttcccatgtatgtgtgaactctctactatagtagcttttctagtatctgagttgacacagctctccctgcccccagttcacatatacactataccatgaaagagaaaaaaaccttaaacacagttatacttttggtcacattgaaattttggcattcctaaatggagagtgttactattatgaaagccaaaacagaactgaagatcgtgacttctcttccttaacctccattcccccattgaccccagggctgaacctaaccagtttctcctcacccttatcctggtttcagtttctcccattcttatcctgggctgattgcaaacaccgtttactccttccccacagaacacaaaggggatatgtttgaacttctatgttctgtctaaggaaagggagaaagaaataaagaagatgaaatggaaagggaaggaggaaaggtggcaaaaacatttctccaccatttgaatttagttggaacagtgtatggctgagagtagaagagtagaagttaagagaggagaatttggtccacctacaagatctccttggctacattaaccttgatcctgctaatgaaagtttaagaaacaaaggaaaaaaatagccaataactaagaaaaattgttcttctcgcagccctgcttgatacaacaatatggaggcttccagagattactggatgttactcaggggcatgtatattacagtttgttgagccctactgtatgctatagttggaaaagggaatgttacagaatgatggtttttaaaaatagttttcctaaatgtagaagaaatgctgaagtaatgtgtttgtgaattactttggaaaacatttgagttagatctgagctgtatcccaaaattttaggtatactaaggatggatttatataacaaaggaaatgatttgctgacttgtgcaaagtgcaaaatgctgtattttggcctaattgggcattctggaaccggattaagaacctatttttcctatgtgaaaagaccttaattaaggccagtgtctatttagaaggttaagtgttactgatagtggtgcctttgtttggctccttcatccatgtaaggacccagcaactacaaatctacctttgaactaaggtttcattcattcattcaaccattcattcattcatttattcaataaatactatggctgttgagcaggaatccgttctttctatttaactctagctggatactgttttttataaacaataactgagtttacctcttggaagcaaatctcattactacttgttgactacttactgaatgcagaacactgagcagagtgttagcacaggctcagaataatcagaaacagtcgcccttccttttgatgaaagtgtctggttgggggaaacgaattcaaagcacttttgtatccaagctaccaattggccatatgaccttggacaagtcactttaccactgtaggcccagtcatttcactgtaaaatgatgagaattgaggtcgttgacttctgaagtgttttaaattacagaaagtctctgaaaatagaatgaacttgtttgccctaagaaaaatattccattgtttactttttattgtgatgttcatatcactgaaaaaatcactgtagttcgactatagttatttcagtgttttctttttttttgagacagcgtcttgccctgttgtcacccaggttggagtgcagtggcaagatcttggctcactgccacctctgcctcccagctcatgcaatcctcccacctcagtcccccaagtagctgggactgcaggaatgggccaccatgtccagctaatttttgtatttttggtagagacagggttttgccatgttgcctaggctggtctcgaactcctgagctcaggtggtcgccctcctaggccactcaaagtgctgggattacagacatgagccgccacacccagccatttcagtattttctaaagttattttgggatatgcataagaaactaaacatcttcacctacacttgcagggtttgcaaatttaaataccttcagggtcacacaaagaacataaatgaggttcagtgttacacaattgggagtggtggggtctgtggcaaaatacatctgcacctcctaaaagtattcaaattcacattgaaacaaaacaagtaaacaaacgaaaacctacacttctggccaaaccagacataatttaggctagagtggttctacaagctgcctgtttagagcaggctataatttgaacacaaatttttaaatgctaattagcaaaatctctataattactttcagatttatagttttttttttttctttttcttttctttttttttttttgaggtggagtctcgctctgtcacccaggctggagtgcagtggcacgatcttagctcactgcaacctccgcctcccaggttcaagcgattctcctacctcagcctcacaagtagctgggattacaggcgcacatcaccatgcctggttaatttttgtatttttagtagaggtgggatttcactgttttggccagactggtcatgaacgcttgacctcaagtgatccacccacctcagcctcccaaagtgctaggattgtaggtgtgagccaccgcgcctggcctacagttggcttttaatcatgtgatacgtgtatttgtccctttatgtttcggttagcacttttaatttatatacagtcatgcatcagttaatgactgggatgtgttctgagaaatatgtcattaggtgattttgtcattgtgcagacatcatagagtatacttccacaaacctagatggtataatctacacacctaatctatatggtgtatcttgttgctcccatactacaaacctgcatggcatgttactgtactggatgctgtaggtaactgtaacgtgatgctatgtgtttgtgtatctaaacataactaaacatagaaaacaacagtaaaaatatggtactgtaggccaggcttagtggcttactcctataatcccagcactttgggaggccgaggtgggcagatcacttgagcccaagagctggagaccagcctgagcaacatagggaaaccccttctctacaaaaaaatttaaaaaattagccgagcatagtggtgcatgcctgtagtcccagctactggggagtctaaggtgagaggatcatttgagcccaggggcagcggttatattgaaccatgattgcgccactgcattccagcccaggtgacagagtgatatatgtggtccactgttgactgaaacattgttatgtggtacatgactacttcagatttttgctatgttttaaattttaattcttacaaatgaagactatacctttgagaaaggaacaataggtagtgcattttgaacattaggaacaatatacagttactgaatctggatggagatgatttttggaaaatgtagtgttaaaagatggatatactaagttgctcttgaccaccataggatgtggttgtacagatggcaaaattttgtaaagatacagtacatctagatctcttaaatgactggaaatcctttgggcctttctgttggcttcttgatagtaatgtttggcaagatactgcatgcatatatattcctgtgtattcagcagatattaccagtagaaatgcaaaaatcttaaatagccaatatttgaacctaacaccagagcaagctgtaattcagtattactataatattgccttttctttgtgtgtgtgtgtgtgtgtgtgtgtgtgtttgttttctttttgtagttggggctacaggctcacaccactagactcagctgtttttatttttttgtaaagacaaggccttgctatgttgccttggttggtcttgaactcctggactcaagcaatcccccaaccttggcctcccaaaatgctgggattacagatatgagccaccatgcccccccccacccctttttcttaactgtagtggttctttgcgcaagcatagtagacaagaattttttgattcctcctgggaactatttcctcaaatttcatggaaactgaattgtcatgagttaagtggaaattagtcttctgatactttttcctttattttacaagttcccatcccaggtttcttatactgaattccttatctctgcctcattcttgaagagggaaatggaagtggggttttggattctaccatgatagaagggaagacctacaacaaggctattcagcagcctttgttttagtgaaagccaatgagaagagatcacttcttccacttactaacaatgattgctcaactaataagtttagcttttttatttcaatgtaaaagttgctgaaaacactgaattggtaaaggaaatttctggatttacattttaacctgatgttgttcaataaatatttgaatatctaccttatgctaggcacagtaatagtctctgaggaatataacagtaaataagagctgaggctgttaatgggagagaagaacaatgtaaatgatcaaacaaaataccatgtggtaagacctgaaaggtcctacatgagttggtctccggtgtactctctaatctaacctcacctagtcctctttccactctgtctccaccccagtcactctggcctcttgttctttgagtacagacaaccacatttgcaataagacctttgcaccagtttctgttattgccctggaactttttccccacatacctacatgactgtctcctttccttcaggactctacccaagggtcttctcatcagaagctcttcttttgttaccattctaactgaaatagtacccctactgtcactgtttactccttaccctgctttgttcttagcaaattttgttattgtctgtcttctccctttctttgtaagctgtgttcaagcacggagtttgttttcttcgttgctgaatcattattggagtatatgcactccattgtttgttaaacaggtgaacacaagaattgccattatagggcaggtatagagtgctaaaggactgcatagtatggccacctaacccagacatggggtgtcttcaggaaagcctctgcaaaaaaaatgtatacacagagggcttaacggatgaaggagagttggaaaggtggggaaagtacaagttcatccaggggccgagagagggagctgagggcagaaaaggtgtgtgtgtgtgtgtgtgtgtgtgtgtgtgtgtgtgtgtttggtgggaatagaggtgtatatggcaaaagatgaagctggaatggggagggactagatctttggggctttataagctaagcctcattaaggtggttgctttagagaagagatcaaagggtactgggaactggaaatgggattggaaaaaggatgaaaggttttacttttatgtgggatgacatagttacaactctctcccccttcctccccacacaaacacattccatagtgtgtgataaaaagatatcattttaccagaggcagttttctcctggatccttaacacattaggaaatattttggatgagcatactagttgaaaatctgaaatggatataagacttttctgggaacttaaatttatgtaatgatgcttctcattgttaatatctgactttaatttcttttggtttgacttcttgccaacattacaactatgtcctttcttcaaaaaacagggtcttatgcttatgagtgaaactgacaaaacatgtgggtgagcagttctataaaaactctttttttttcagtaacatcttggagggtacaaagggctttcacataatcttatttgacacaacaatcctgtgaaacagatggtattatctctaattttcagatgagggaactgaggttgagagagatgaacctaattgtataatatcacatacaaaattgcatctctccctggtcttttggctctaaacctatttctcatcacatttttctgatctaaggctatgattattatttgaaagtctgctaccccttcttattgagtaaatattgtgttaagttctatgtaagatgccaaaactaagggcctgtctcgttttccaattgctatggtcttattaaagaaatgaggcatttcttattaagaaatgcttacctaaaataactgaagggctgagtcaaatctgtaatgtctagacaacttacagggtgctgcatgaaactcagaatattagagaagcttagtggagaaaagcctctaaagaaggtaataactgtgtccgaagaaacaaatagggataggatgtggaagttgaaatttattccagacagagaatgaaggaaatatgtaaagtgtacagaaaagagatttgtatggaatggagagtgttaaatgagaataactattgatacttcttttgtacttggataaaaattgtgcattgatgaattctgggtgtgtgtgtgtgtgtgtatgtgtgtatgtgtgtatgtgtagggttattcctgatttagctaataaatgtgatctttttggaaataatggtgaaagaaatacctagccgagttgcttggacttacaggttatttcctaatttagctaataaacatggtagttttggaaataatgctgaaagaaatactagcctagttgcttgaaactaggctaagtgctcaagatagtgaacacaggaagagaagtggtctaggaaaaagattaatgagttaactattaacatttatgtttacaattttttttttatcagagaggtctgctgaaatggagatggtgggtggcgaaactccttgtagatgtttttacaaactcacatttgaaatctaaataagttacatatctcttaagaaaatatcattcagtttttgcctgacatgatcaataagatgccacctttttctcagattagctagttttaaaaggtaaagaagcttcaaggcctagcttagagtttgagtccttcagcaatctctctctaattcacttcttcctaagtctgtgctgggaccctctcctgtgtgttctccatcaccgtatgtccctttattatgattatatattcatatatgccttcctttcatctaaaggctgtatctgtctttcccactgctgcatccccagggcccagaaatacgtatttgttgatggagtgatagaatgagtgaataactttgtgctgtcactgtttataactggttgatgatttggttgtctgaaagcatgctcagtgctctctcctttgactagcttttttgttttcatcctgtaatgtagactgtgtggtaaaatattgagtctagtttcactgatgcaggtaatgtagagggcatcttttagtaatctttgcttcaagtgagaataatttgttgtgaaataagcctactaagctgcctacttggctgctgaatgattcttttacccctaaattagaacagtctggtctgcttaccagtaagatctttggcacatgtatttcaataaaatgagcccttgtcttatcttactagtagtgctgtgtgagttgtcctgagtcattcagtggtaattgagtgcctactatctatcagacactgttctcggtactttagatactacagggaacaaaaaataaccctgtcctccacgaacattgtacaagcagataacaataaacaacagtgagcatataaaaaaggaaattacatagtctgttagcagacaataatgctataactaaaaagaaaaggtagaaaggtagaacaggaaaggggaattaggagtacaagggtggggggaagatgaaattttaaatggataggccttattgagatgaattttgagcaaaagtgtgaagaagtagaggaattagacctggggatatctagaggaagagcattccaggcagaagaggaagtcagtgcaaaggtcctgaggcagactgcatgatatattggaactggagcaaggaggccagtgtggagctgagtaagagcaagggacagagacatagactatgaaatcagagacataattaggggcagtgtactggattaggtagggctttatctgccattgaaaggaattttgtgttttactcttagtaaaatagggagccttttgaggaaaggagtgccctgatctgagttttatttctaaagaaacactcttcccctgtgttgagaatagactgtagtaagtcccaaggatcaataggtgagttgcagaagcttgtgcttacaaattgtcaatccctgtgattgttttaagcaccctgtttaaatatggaataaatgggtttttttgttttgttttgttttttgccttttgataattgctttgaataggactgatagtagtaagagacgccagtctatagatatcgaagagttttttctacataggttctttctgattgttctgtgctttaaaaagtgggaggtggggaggaagagggcgggggcagtggtgctggtcttaacaattgggtattatccaagaagtataaatgaagctgtaaaatggggtgctgtctagagtactgaaaagcatttatgacagagttcttccttcttgctgtcacagtacattttactcacaggagtcacattgagaaatgtgatttgtgctctctatactttgattaaatttaaagaagaccaacttcacaagaaaagtgaaaaacaagatttgtatcagtccatgaaattgttttcagcttttagcctaataaagtccaacttaacttaattcctttgtaaagatgtgagaactagatagctttccaaccatactcaagtctaaccttccttaatttactttaatttactttacttaatttactttaggtgttatatacattgacaatattaggcatgtctttactaaaatgtaattgactatgctttccagaaacaaatactatagaaattatgtgactctccattaatgcctcttttggaatgtgcttcttaaaatgtgctcatttatattccttgccccccatcctgttcattatttttagaagttggtgcatcttccataaacaatcagcaactttgcatgatgcttgcttttgtcccctgttgtaagacttattttgaaaatctttctatatgggagttagaatgtaacaattggtggtcatggattggtctttgggcctcctggaaacaagtatagtgattaagttagttcctgcaagatgcttctgccataaataatgcctttcagtttatttcattgtagcttagtagtaatgcttgatagttattactatatagactgattaatcttataaacttacataatgttttggcagtctcaccctcgttctaaattttggtgataaaatggatgtggtctctgagggggaaaggatttgagagaaattacatagtggtaaactgttctaattaaaagctctttccctctctctctctctctctctgttgccatgcagtcagtggtcctctggctgcctaagatttcttaactcactggtaaccatttacacataatagctgtacttgaagttgctatggtttcattagtagttttgcctaacctttgtgaactggctcaggcctattagttaggactagtattatttttatttaaaaataagttatgtttgtctcttctccataaaattatttagaaaagttcaaagaataaccctcttattatccaatgtcaaaaaatataattatgtgcaaagtagtcttatatgaccgaaattagattctgtcttcctcagcttatgcatcaatttagggttaaaaaatacagtaactgtgattaggacagaatactgctatgttcttgccagcctgagaactttgttaagattaactttgatagtaaatacagtattgagaattactaaggaaattattcagacatattgcttccctagttattgtgacgtttaaagagttttaaaaaggtcattcccttgatgtaaattttgtatcagctgaaagaattgttggaagaatatgtgctgtgattaagatcatggtctgtggggtcagttagttttagatccaactcctgacattactactctttttgaccttgagtagatggcttatccttattctgtctctatttccttgtcaatgaaatgggaacgctaatggacattatctcacagagttattgggaagagtacatgagacataatgagtacatggacataataaactgcttgctgcagtgctggacacagaataagtgctagttagtgcctttggttatgttgtgttgagattggaacatttcttaaaacgtttcagtctcatttacttattaattacatttagagtctactctttcctcaaggccgtataaacatgcaagttttaaaaatatgaaatgctaacatcttaaggttgcaactaggttaagtcctgttgatcttcatgaaattgagaactgaccatggcagccctaccaggaaattatgacagagaaaaaaattccattcctcttggaggacagaaagtaataaatatagtgtattagttgtgtgtgtaggtatataaagagaggtcacacacacacacgtttattcttataatatattaataatagatctaaattactgacctgctttcttccctccaaaattcataaaaaaatgaaaatagaaatgaaatcagcattctttatagctcagctgatatatattattacccaatctcaggaataggaatgattcttagacagtaaataaaacttacacttatttttaaaaattcagcctcttagccaggcacggtagctcactcctgtaatcccagcactttgggaggccatggtgggtggatcacttgagatcaggagttcgagaccagcctggccaacatgatgaaaccccgcctctactaaaaatacaaaaattagccaggcatggtggcgagtacctgtaatcccagctactcgggaggctgaggcacgagaatcgcttaatcccaggaggtggaggttgcagtgagccgagcttgtgccactgcactccagcctgggcgacagagcaaggctctgtctcaagaaaaaaaaaaaaaatcagcctttttagaaccgatctactttctatgtttccatagctgtgtaatacagtaatgagtgggttaaggcattgtgtctagaatatgtgtggtgaacctgttggacttgaatgtaatcattccctaatcttaccacataaagatgggattaataaccagcaccacacctgtgaggagaaattctggaatgcctcatcttgcctccagataaaacatgttcctccttcctttaaggtaaatgcaggaatattgtcaatgaacaaaatcctttcactagtcattcctgaaaaagaattacagttggaaaataaaaaaaatactcctattttagaacctgtaatgtggaagacttataatgaggtgaaaatgtagcggtggatttaaaactacaagatacttaattatagccatgcagtaagttatactatatatttgtcatgcaagaataacatttgtaagaaaactgatactgacacatgtatctttctaatccctacctttgctgtttttgttttcttttctcttttattttttgtagagatggggtttccctgtgttacccaggctgatctcacactcctgcctcagcctcccaaagtgctaagattataggtgtgagccacaaagcttggcctggtttcttttttcttgagataacctggaaaaagatatgtcaagtaggaataagtatgtaaatgtttagaacagatggcaggtagttaaagttagaatagtaatgttgggaggctaaattgggaggatcacctgagaccaagagttagagcccagcctgagcaacatagtgagaccccatctctacaggaaaatttaaaaaaattagctgtgcgtggtggtgcacacctttagtcctagctactcggaaggctgaggtaggaggatcacttgagcccaggagtttgaggttgcagtgaattactatgattgctccactgcatgacagagcatgaccctgtctctaaaaaaaagaaaagtaaaagaatagcgatgttgaaaatgaggtaatgaggtgcccttccccccaaaaaatgagtagttgttagcttttagctgtcatcgtggtacagctaccattttaagggaagggtagccttcccttaaaaacttagaagtcgggccaggtgcggtggctcacgcttgtaatcccagcactttgggaggctgaggcggggggatcatgaggtcaggagatcaagaccatcttggccaacacggtgaaacctcgtctccactaaaaatacaaagattagccgggcatggtggtgggcacctggagtcccagcaactcgggaggctgaggcaggagaatggcgtgaacctgggaggcagaggttgtagtgagctgagattgcgccactgcactccagcctggcagtagagcgagactccatttcaaaaacaaaaacaaaaaccaaaaaaaccaaaaaacttataaaagtcttctgaagataagaactttcttaggggttctagattgttatgtaacttttcactaatttctaggtatctggtgacccatctcatgggggcagatctgaacaacattgtgaaatgtcagaagcttacagatgaccatgttcagttccttatctaccaaattctccgaggtctaaaggtacagataatacaagtaataattttttaaaatgaattctccctttctcccctccttttagggcttaaacaaacaagtaaacaacttttctaatggaaaattcaaacacataaaatcacaggtaaaggtcatatagtacagtaaactgccatgttcactcagtgacaacagttaccgacatatggccaatcatgtttcttttgtatttcccaagccctggattattttaaagcaagtctagacattatataataccatccatagatacttcagtttgcatatctgaaagataaggactcttcattcttacatctaaacaataatttattgatgtaatctaatatctggtcagcgtaatgttgagattttcaaaaaatcctcaagaaagactaatttatttgttggaggggcagataaacagtgttttaaaattaaacccatctttgatatttagctgctttgagtccttattgagctccagagtgccaaccagaaaatagattaaaagctcaactgagtggagtcaatggctgtggaaattatagtaatggtaaatgtacacaatactgatagttacttatgctgatttctaatcttactattaacactagcagttcttactgattcatgaaaatgtgcagcaaatatgttatataatatgacaatagaaggttggaggtaactttgacttttttctcttttgcagtatatacattcagctgacataattcacagggtatgtattgtgactttgattacattattttggggaagtggggtgagagtggaagaatggcatttagccaagatcctaatctaaactttagccattgaaatgttgattgattgcaactttactgtaggtttaagctgattaaagaaagaagatgtgtagtttttttctatgtaaggctaacttggactggatattgactgtaggatggagattgtcttttgatagctggatgaagtcttttatgtctggatcctcattattacctgtagggggagaattgatcatgcatcataaagttgatcctgtcttccctgtatttgcttcctaggacctaaaacctagtaatctagctgtgaatgaagactgtgagctgaaggtaaaatgaagagacagtattcattgtttgctttactttgagattatatgtttgactaagcaggcagactttcttagggagtagctttgtgagccatgactatctgaaattcgtattatttttattatcagactttgaaatttaaaccatttctgagtaagctaagtgacattgaaagtggcatcacttgtttttattttaacataaagttaaattataaatttgatatattagtcataataaaggaggataatagtcaaatatcaagtaagtaggcccaaagaggtgataactagattaaatacatttctaagaggagaaatggcagaaacccactcactcaaaataattcaaataaaaaataagtgaattaattgtttctaaatagcaagcagctctagaacttttgttccatttcatttttttaacttgaaaaaatgtctgtacattagaatgcaccatttcatttggcaaaattatatatccacttaactcacatttatgataaagaccatttctttttttttccttttttttgggatgcagtctcactctgttgcccagtctagagtgcagtggtgtgatcttggctcactgcaacctctgcctcccgggttcaagcgattctcctgcctcagcctcccgagtagctgggactacaggtgcacgccatcacacccggctaatttttgtatttttagtagagatgggatttcaccatgttggctaggatggtgtcgatctcctgacctcgtgatccacctgcctcggcctctcaaagtgctgggattgcaggtgtgagccattgctcccagccaagaacatttggattactttaaaaagtttatggccaggcgtggtggctcatgcctgtaatcccagcactttgggaggccaaggcgggtggattatgaggtcaggagatagagaccatcttggccaacacggtgaaacctcatctccactaaaaatacaaaaatttagttgggcatggtggcgggcacctggagtcccagctacttgagaggctgaggcaggagaatggcgtgaacctgggaggcagagcttgcagtgcgccgagatagtgccactgcaccccagcctgggcaacagagcaagactccgtctcaaaaaaaaaaaaaaaaaaaaaaaagtttattgtgtcccttcctagtcaatccctacccctccacccctaactctggctccaggcaactatggatctgcttctaacactatagattaggtttatcttttctggagtttcatgtaaatggaatcatatagtgtgtatttttgtgtgtctggcctttgcttggtgtaatgattcggtattgttgtgtgtgtcagtggtttcttcctttttattggtaatagtattttgttttatgaacataacagaatttttaaatctatccacctgctgatggacatttgcttgagtttgaggctgtgtaaagcaactgtgaatgtttatgtgcaaacctttgtatggacgtaagttctcattgcacttgattaaatacctgggagtgacatttttgttttgctttttgtaatttcattttaagttcttgctgtatatttaccatacattctaagtatggaccaggaaatctctttatgaaagtgctattttgttctcctttttaataaggcaacagaggtttgtttgttgttgttgttttgtttttttttccctgcctgtttctaagtcttagcagtttgtctgttcctcttctgccctttagattctggattttggactggctcggcacacagatgatgaaatgacaggctacgtggccactaggtggtacagggctcctgagatcatgctgaactggatgcattacaaccagacaggtattactcgccttggttatttagggccttatttaattccatgttggatgcatttgggattctcagaaatagagatggtgggagtgggaagaaatgtcttttcttccctcagtgatccttttaaagcctgcaagtaaaaatatttcacttcctcagatggggaagtggggttttatacacagcttttaaaccttgtgatttagcaattgggtttttatttatttagattttgaatatattctctgagtacttatacctaaagacaattagtccataattttaacccctcaatgttacttggaagaatctaccattctactaataagaaacttgcccctttttaaacagcttatttctcttaagtgtgttttgacttaccccacctttgaacagccctttagtcaggtgaagatcttagtaagctatttatttctgtaaaaatgagtatgattgttgagcctcagatgtcttaaatagtaatttgaatatttcacccctagttacggtttcatttgttgccaagaattgtaacagtgacattgcatatacttttacttctttttaattttgccagttgatatttggtcagtgggatgcataatggccgagctgttgactggaagaacattgtttcctggtacagaccgtatcctttaaaaagttttggattcttgtttcttatctgtattccagtgtccatgggtgtatactccttttacttatctctagggaagactctcagtattttgcttttatagtctagataatgcattatgaggtctttcttcaaactgagtagcttgccttgccaagaaagaggcttcagtttacttttgtgagttggaaaaattcctcataagttgaaaaacccatcaagtctgtcttctttttgcaacagtcccttattaatcatcccacagcagttctgagacctgagcatactcatgagaaactccagatcccagtgacagttttagtcttcttgtataaacacttaatgagcacaagatgcccatttataaatgaagggcatcttgctgttatgtttatgtattaaaagggctatgtttggtacctgcagtgatatactgcttacttgttatgtttgttgtttgacaacaaatagtaaaagcaagaagtgaaaagcttaccatgtaatgtacttttgtttttccagcacaactttttcccagattgcagccaaaccctaatttactccaagacttactgtctagtttatggtttattatagcccctttattttagctcctttcctatctgcggtatgcttgtttcatggtttagtattaatgtcatgaaaaatgtggtctgttggaactctcatcagttctacttatggaagatgtttaggtgagaaagattgttaaaatatatgtctagaattagttttggggattttttctttttttaatagaattgagttaactagccttcaggtggttttggttaggcaatattagacatgctacctctgatctattatgcctagagatctaaaagccattttagtcccttacctaaataaactaattgagtttataaaccagaatgtcaattcacttgcttacagtttgggatatacttctgtaatggctatgatgttcatgatatctacccacgtgttctaactatcttctgctagagagaatgtactttcattatgaactgaaaaaacctctatttctcaggtaaagctgtttctttctctctgaatattaggattgataacagagttcagtctccctgtatttagcttaccttattactccctacctctggattttaaattagtttaatacctggtgtttctcctttgtcctttgtttcattcaccttatatctgtttctcagaaatttggtatatatatattacagtccaaggtaagagaattagggaaaattcatacttccccctttcctcaggtacatatctcagagtcccatccttgctttgtcctctagagtagctgctgtcctgatgctattctctgttccctgctccccttcctcttcttgtgtctgtatctccagctaccatttagtcagtcacagacctccatgccggtgtagacacacaaggtggcagagagggaaaggcagtataacatacaggttaagcacatgctctggagccagatttcctggatttagatcttagctcttttatttccagctatgtaactttgagtaagtttcctaacctctatttctctcagtttccttaaccgtaaaatgataataatcatacatactcacaaggttgtcgtaagaattcagccagaaaataactggaaagtactgagacgagcctgccagtaaatgttagccatcatgttcagtaatggccttgcaaaacagattaccccttcaccttctcacttaattgtctacctatgaatcattaatgttttgttttgtttttaattctgtgataggtaggaaaggatggaactccttggcagactagtgttagaaagttttcgaagcagtgtgagtcttgtacctttgtggtcctgtctcacagacacctgtctattccctgacccttttaaatgctaactttctgcctgtaggaaatcttccctttgtgcttaggtctttttcttctgtgagctttagataaacaacctagtgtttaaactttttaataagggattcattttttaatacatgagaattcatttcaaaattttggttttagttatttattttattctacttggctctttttcagacagatgttctctcctggattgtaaaagtcgaattcaaaggatttttatttgtaatatacttaacctttctcttgtaagttgccatctgtgtagatacagctttgattgcctgacaagaggaaaatgtttcccattatcttttcctgcctgaactatacggtcacttgtgttccagcatagtggttcttaaccctcatagtgtgtcagaatcactttgcagagcttttaaaaactctagatgcctggggaccaccccaaagactccattttgttgtcatgggtcaaagcacagtcttctagtttgcagctagtgttgagtacaactagagtttaacccagttgaattttagtttaatcttggctggtcttgaagatgttagtaatctctattcattttttttgaaaagtaccaatgagatcagaaagttaattagaaaacatctagttgaatcccctgtttttaatagatggggaaaccaagacccagagaatataatccaaagctacctgtcacataggccacaatttcttttccaatattctgttcttcgctgttcttctaatttgcagaactcctctttaaaaaacctttggagaatgtattggcctcataccctcttccttcagcctgaaagacatgcacctgtcacttatttatgatatttaaatgcaacctctagaacaggggtgtccaatcttctggcttccctgggccacattggaagaagaaatgtcctgggccacacataaaatacactaatgatagccgatgaacttaaaaaaaattgcaaaaaaaaaatctcgtaatgttttcagaaagtttacaaatttgtgatgggctgcatccaaagccctgggccacgtgcagcccgcaggccctgggttggaaaagcttgctctagaaggtactcattgtttgagtgtccagttttctccctgttttccttatttattgatcattttgcatttggaattggtggtttgggcttgtaagtaagaactgtgtactagaacttttgtggtattgcaaatgttttatatctgtgctatccaatgtggtggttataggcaccagctgcctgtggctaattattaagcaactgaaatgcagctagtggtgagcagtaacgttgtggagaagaaaaaaaagaaatgtagctagtttaacttggaaattgaattgaaaatatttttgtggttaaatgtatttaacataaaatttaccattttaacctttttttttttgagacagtcttgctttgttgcccacgctggagtgcagtggcatgatcttggctcactacaacctccgcctcccgggctcaagcgattctcctgcctcagcctcccaagtagctgggattataggtgtgtaccaccatgtaatttttgtatttttagtagagacagggtttcaccatgttagccaggctggtctcaaactcctggcctcaagtgatccgcccaccttagcctcccaaattgctagattacaggcgcgagccactgcacccggccccattttaaccattttaagtttacaattcagcggcattaattacattcatgatgtacaatcactactactacctatttccagaattttttcatcaccgcagatggaaacactcagcccgtgaaacagtaacttcccattctcccctctggtaacctctgttctttctgtctctgtggatttgcttattctagaaatttcatagaagtagaatcatattatactgtatttgtccttttgtttctggattatttcacttagtataatgttttcatggttcttccatgatgtagcatgtatcagaatactattccattgtaagtatttaatatatcccacattttgttgacctgttcatctgttagtagacagttgagttgtttttacctttggctattgtgaataatgctgcagtgaacattgtcatacaagcatctgtttgagtccctgcttttgattcttttggttatgtacctaggagtggaattgttgcatcatatggtaactctatgtttaactttctgaagagccaccaaactcttttccatagtgtctgcaccattttacatgcccaccagcaatgcatgaggattcctgtatctccaaacttgttctttttttttttttaatagtcacccaagtagttacaaagtggtatctcactgtggttttgatttgcatttctctcatgactaatgattttgagcatcttttcatctgcttattggccatttgtatgtcttctttggagaaatgtctattcaagtcttgcctattcttaattaggttgtttgtgtttttgttgttcagtggtggttctttatatatcttagacccttatcagatacatgatttgcaagtattttcacctattttgtgggttgtcttttcactttcttgataatgtcctttgatacataaaacatttttaattttgattaagttccaatttattttttttctgtccttgctcatgcttttttgatgtcatattttagactgtattgccaaattcaaggtcatgaataatttacccctatgttttctactaagagttttgtaactactaggaatataatagggtttagctcttatacttaggtctttgatccattttgaattaatttttatagatgatgtgaggtaggggcccaacttcattcttttgcatctggaagtccagttattctagcaccataaagagactattacttcccccatcgaatcaacatgataccctaggatttctcctttgttttttgtttctttcaccttttacctgtttcttagaaatttggtatatataattagcaatacacgtgtgggtttaattctggactctcagttctattctattggtttatatatctatccttatggcaataccacactgttttgatcactgtagctttatagtaagctttgaaatcaggaagtgtgagtcttccagctttgttcttctttttcaagattgctttgactattctgggtcccttgtaatttcatatgaatttaacaattggcatttctatttctgcaaaaaaaaggctttagaattttgatagggattgtgtcagatttttagatcactttaagtgatattgacatcttaaccactattaagtcatcctgttcatgaacaaaggatctctttccatttatttaagtctttatttttttttcagcagtgttttatagttttcagttgtaaagtattttaccttggttaaattttttcctatgtattttattatttttgctgcgattatagatggaaattctttggtgtgttcattgttgatatatagaaaccaaattaatttttgattgttgatcttacaccctgcaaatttgctcaatttatttattagctctagcagctttcttctagattctttgtgattttctatgtatagggttatgtcatctgcaaacagagatagttttatttcttcctttccaatttagatgcttttaattctttctcttgtctaatgtaatggctctgtctagaacttctgctacaatgctgaatagcaacagtgaaagtaggcatccttgtcttgttcctgtcttagggggaaagctttcagtctcttcccattgagtatgatgttagctgttggtttttcataaatgccctttatcatgttggggaagttctcttctttggtagtccatttcacattgctatgaaggaatacctgaaactaggtaatttatcaagaaaagaggtttatttggctcacagttgcatagctgtaaaagaagcattgtgccagcatctgtttctggtcaggacctcaggaagctttcacttatggtggtagggggaacaggcatgtcacatgtcaaaggagggagcaagagagatgccagactcttttaaacaaccagcatttgcatgaactgatagagcgagaactcactcattaatgtggggagggaaccaagccattcatgagggatccaagaccctaacaccttcaatcaggccccacctccaacattggggatcacatttcaacatacttggaggggacaaaacatccaaaccctatcaccttctgttcacagttttctgagtattttaatctgaattttaaatgccattaaaattttatttagattacattgaaacttaattacacatggctagtggtattggtttatgcatttttagatcccttcaagttatcttcctgttctacctttttcttgggttttgtaattagtctccttcttgaaagattattttggtactggttgttagtaattcctggatttcctttaaagatataagaaatactggggaggggacaagatggacaactagaaatagctgcaatcagaggcttccaacaagaagaacgaaaacggtgagtgaatcctgcaccggcaactgaggtattcaccttctctcaatgggactgactaggtggttggcatgacccatggagagtgaggaaaagcagggtggagcgacagcccacctgggagctgcaaagggcaaggggagctcccaccccaagccaagggaggtggtgagtgattgttctaccctgtccaggaaatcatgctttttccaaggatctgtgcaacctgtgattcaggagatccccctcatgagcacatgccaccagggccttgggtcccaagcacagagctgtgtaggttcttggtagccctttggctggagactacataagactactgagttccttgggggaggggttgctgtcatcactgcagctccagtctgctgttttcccctgctggtgctggagagactgggcggttgggacccagaaggaattccctacagtgcagaccctacagtggtggtggcacatcatggccagactgactctttaggctgaactctgactcatccccctacacggggcagggcctctctgcgggaatttcagcaactccagccaggggtttacagacagaattccaatctccctgggatggagcccctggtgggaggggtggccacagtctccacagatcagcagagttagtctttccccttgctggctctgaggaatccaggcagtctggatgagtgagatttcccccagtgcagcacaccccctccaccaaggggcagccagagtgcttcgttaggtgggcctgtgcctcttgactggatgagacccctccaacaggggtcgccagacaccttatgcaagagcattcccactggcatcaggttggtgcccctctgggacggagctcccagaggaaggaacaggcagccatctttgctgttctgcagtctccactggtgacacctccaggtacaggagggacccaggcaaagagtctggagtggacccccagcaaactgcagcagccctatggaagaggggcctgactgttaaaagcaaaacaaatggaaagcaacaacaacatcaacaaaaaggtccccaccaaaacccctccaaaggttagcagcctcaaagttcaaagctaggtaaagtcatgaagatgagaaagaatcaatgaaaaaacgctgaaaactcaaaaagccagagtgcttcttttcctccaaatgatcacagcacctctctggcaagggcatagatgtgggctgaggctgagatggatgaattgacagaagtagacttccgaaggtggataataatgaatttcactgagctaaaggagcatgttctaacccaatgcaaagaatctaagaaccatgataaaacattacaggagctgttaaccagaataaccagtttagaaaggaacataaatgatggagctgaaaaacacaacatgagaacttcataatgcaaccacaagtatcaatagccaaatagaccaagtggaggaaagaatttcagagcttgaagactatcttgctgacaagataagagaagaatgaaaggaaatgaacaaaacttttgagaactatgggattatttaaaaagactgaacctgcgaatgataggggtacctaaaagaattggggagaatggaaacaagttggaaaacacacttcaggatatcagccaggagaacttccccaacctagcaagacaggccagcattcaaatcaggaaatccctagagaaccccagtaagctaccccatgagaagatcaaccccaagatacataatcatcagattctccaaggttgaaatgaaggaaaaaatactaagggcagccagagagaaaggctaggtcacctacaaagggaagcccatcaggctaagagtggacctcagcaaaaaccctacaagccagaagagattcggggccaatattcaacattcttaaagaatttccaacccaaagtttcatatctggccaaactaagcttcatagtcaaagaagaaataaaatcctttttagacaagcaaatgctgagggaattcgtcaccaccaggcctgtcttgcaagagctcctgaaggaagcactaaatatggaaaggaaaaacttactggccactacagaaacacactgttttcttttaccagtaacactatgaagcagttacctcaacaagtctgcaaaataaccagctagcatcatgatgataggatcaaattcacacataaaaaatattaaccttaaatgtaagtgggctaaatgccccaattaaaaaacacagaacggcaagctggataaagaatcacgacccatcagtatgctgttgtattcaagagacccatctaacatgcaaagacatacaaaggctcagaataaagggatggaggaaaatttaccaagcaaatggaaagcagaaaaaagcaggggttgcaatcctattttctgacaaaacagactttgaaccagcaaatatcaaaaaagacaagggcattacatagtggtaaagggctcaattcaacaagaaaagctaactatcctaaatatatatgcacctaatacaggagcacccagatttataaaccaagttcttagagacctacacagagacttagactcccatacaataatagtgtgagactttaataccccactgtcaatattagacagatcatcaagcagaaaattaacaaggatattcaggacttgaactcagctctggacctgatagatagctacagaactctctatacagaaacaacagaatatacattcttcttagcagcacatcgcacttaggctaaaattgatcacataattggaagtaaaacactcctcagtaactgcaaaagaactgaaaatataacacacagcctctcacaccacagcacaatcaaattagaactaagattaagaaactcactcaaaaccacacaattacgtggaaattggacaacctgctcctgaatgactcctggataaataatgaaattaaggcggaaatcagaagttctttgaaaccaatgagaacaaagagaacataccagaatctctgggacacagctaaagcagtgttaagagggaagtttatagcactaaatgcccacatcaaaaagctagaaagatctcaaattgacaccctaacatcacaactaaaagaactagggaaccaagaccaaacaaaccctaaagctagcagaggacaagaaataaccaagatcagagtggaactgaaggagatagagacaggaaaaacccttccaaaaatatcagtgactcccaagatctggtattttgaaaaacataaaataaaataaaatagactcctagctagagtaatgaagaaagaagaatcaaatagacacaataaaaatgataaagggggtatcaccactgaccctgcagaaatatgaacaactatcagagaatactataaacacctctatgcaaataaactagaaaatctagaagaaatggatgaattcctggacacatatgacctcccaagactgaaccaggaaaacgttgaatctaccaataacaagttctcaaattgaggcagtaataaatagcctaccaagcaagaaaagcccaggaccagacagattacagccaaattctaccagaggtacaaagaagagctggtaccatttcttctgaaactattccaaacatacaaaaggagggactcccctataactcattttatgaggccagtattatcccaataccaaagcctgccagaaatacaacagaaaaagaaaacttaaggccaatatccctgatgaacatcgatgcaaaaatcctcagtaaaatactggcaaaccaaatccagcagcatgtcaaaagcttatccatccaccacggtcaagtcagcttcatccctgggatgcaaggctgttttaacatatgcaaatcaataaacataattgatcacataaacagaactaaaggcaaaaaccacatgattatctcaatggatgcagaaaaggcctttaataaaattcaacatcccttcatgttaaaaactctcagtaaactaggtgttgatggaacatacctcaaaataagccatgtaagacaaacccacagccagtatcttactgaataggcaaaagctggaagtattccccttcaaaactggcacaaggatgccctctttccctactcctattcaacatagtattggaaattctggccagggcaatcaggcaaaagaaagaaagaaagggtattcaaataggaagagaggaagtaaaactctgtttgcaggtgacatgatcctatatctagaaaaccttgttgtctcagcccaaaagcttcttaagctgataagcaacttcagcaaagtctcgggatacaaaatcaaagtgcaaaaatcacaactattcctatacaccaacagtaggcaagcagagagccaaatcatgaatgaactcttattcacaactgctaaaagagaataaaatacccagaactatagctattaataacaagggaagtgaaggacctcttcaaggagaactacaaaccactgctgaaggaaatcagagaggacacaaacaaatggaaaaacattctgtgctcatggataggaagaatcaatatcatgaaaatggccatattgcccaaagtaatttataaatttaatgctattcccattaaactactgttgacattcttcacagaattagaaaaaaactttaaaattcatatggaaccaaaaaagagtccatatagcccaaacaattctaagcaaaaagaacaaacctggaggcatcacactacctgacttcaaactatactacgaggctacagtaatcaaaacagcatggtactagtacaaaaacagaccaatggaacagaatagagatctcagaaataaaactgcacatctacaaccatctgatcttcaacaaacctgacaaaacgagcaatggggaaaggattccctatttaataaatggtgctgggagaactggctagccatgtgcagaaaattgaaactggaccccttccttacaccttatacaaaaattaactcaagatggattaaagacttaaatgtagaacccaaaacgataaaaaccctagaagaaaatctaggcaatatcattaaggacatagacatgggcaaaaatttcatgatgaaaacatcaaaagcaatggcaacaaaagcagaaactgacaaatgggcttctgcacagcaaaagaaactatcgtcagagtgaacagacaacctacagaatgggagacagtttttgcaatctatccatctgacaaaagtctaatatccagaatctacaaggaatttaaacaaatttacaaggaaaaaacccattaaaaagttggcaaagggccaggcacagtgactcatgcctgtaatcccagcactttgggaggctgaggcaggtggatcacgaggtcaggagatcgagatcatcctgactaatttggcgaaaccccatctctactaaaaatgcaaaaaattagccaggcatggtggtgtgcgcctgtagtcccagctactcaggaggctggggcaggagaatgacgtgaacccaggaggcggagctggcagtgagccgagatcacaccactgcactccagcctgggcgacagagcgagactccgtctcaaaaaaaaaaaaaaaatattggcaaaggacatgaacaaacacttctcaaaagaagacatttatacagccaacaaacatgaaaaaaagctcagtgtcactgaccattagagaaatgcaaatgaaaaccacaatgagatactgtctcatgctagtcagaatggtgattattaaaaagtcaagaaacaacagatgctggtgaggctatggagaaatatgaacacttttacaccgttggtaggaatgtaaattagttcaaccattgtggaagactggtgaaatcctcaaagacctagaaccagaaataacatttgacccagcaatcccattactgggtatatacccaaaggaatataaatcattctgttacaaagatacatgcatgtgtatgttgattgcagcagtattcagaatagcaaagacatggaatcaacccaaatgcccatcagtgatggactggataaaaaaacatggtacatatacaccatggaatactatacacccataaaaatgaatgagattgtgccctttgcggggacactgatggagcttgaagccattttcctcagaaagccaaacaccacatgttctcacaagtgggagctgaacaatgagaacacatggacacatggaggagaacaacacacgctggggcctgttggtggggtgggggtcaggcggagggaacgcatcaggataaatagctaaggcatgcggggcttaacacctaggtgatggattgataggtggagcaaaccacgatggcacatatttacctatgtaacaaacctgcacatcctgcacttgtatctcagaactttaatttaattttaaaaaaatataagaaatactcatttttgtcttaaagccaataatgtgttatcagattagaaattattcttgtttgtgactttgcagaacccttccatctggcctaatgatagcattattattttttaaagaatgaagccctttcttaaaagcctagttgactagggatatcaagtggacacagctgacagaactttgtactagaaaggagggaggatacagaatgtctcttcaggtacagtactaacttatttcaaataccatagatcttgaaatgaataagccactttgaaaattctgttaccaacgcttaacactctgcagcttaaggaagagagaaagttttatagccctgttatgcatcaaataccgtttttctaccagagatcacagccttggcttgctgttgttttccttttctccataatcaagtgaatttccttatgtgtaaaaccttctttaattttaacagaaatggtgctcttaagacataatgggttaatggagctgagttaaaatccacttgccatcagttactgccctggtctatgggagttttcctagtgtattaaatttgcttcttatatatttttttttatttcttcttttgcaaacatgttttgaaatatttcagacatactgctagacaaagaataataaacacctctgttcatactgtcctgcttgtgagttaaaacactggcaatagagctaaagcttctggagacccttctctcacatctgcccatcactttcagtcattggtttcaaatactctttctttttttttttgagatggagtctcactctgttgcctaggctggaatgcagtggcgcaatctcggctcactgcaacctccacctcctgaggctggtctagaactccagacctcaagtgatctgcccgccttggcctaccaaaatgttgggattacaggtgtggccactgcaccctgccaaatattctccctctctctctttttttttttttttggagacagagtctcgctctgtcgcccaggctggagtgcaatggcgcaatctctgctcactgcaacctccacctcccgtgttcaagcgattctccctgcctcagcctcccgagtagctgggattacaggtgcacgtcatcacgcccggctaatttttgtatttttagtagagacaggattttgccatgttggccagcctggatgttctctcatttttaaaaagcagtttcactttagacaccaaaagcaagtgtccaataaatgctgaagaagaaaaatattttattaggttatttgccctttcctctctaacttgaaaatacattttctcaggaaaaggtagaatcctcagatttgtccataaaccaagggtcccttaagaaattttctttgtgttacagtgaaaatgactattttgacaacttggaagataaggagctaggctaagtcttattctagtctatcctgtctgtcaccaggaagatttcctaccttcccaaaggttagtgctccacttcacccctatgtggatttatgtctttgaaaattactgctcatggcaggtaggtcctgcaattggtccttttatttatcttcaactttctggcctgtggaggttttttaggagtgcctggagcacttagaactgactagaatatccctcttttactttccctagaaatatcctgttatgcttagtttttttgtttttttttttctttacctgttcattctgactgacttgcttctgttaaagatgacttcgtctaaagagtaatcttaggcttaagttgttcttttcttcatttgttttagaaagattcaggagatttatgatttaaattttgacgttgtgatcattacaattaaaacacattttaaactaatagtaaaaatattttctacctaagagaaacagggctaaacctattacaaaggatggctcactgtggaaataactgattgtcatgatgtggtgagttttgctttactccgctaatgtggctacgtgacgctccttctggtcttaactgcctggacaaatcatagtggccttatagtagctggtactgttggaagactggcctgtatttcagtggatttgccagcttttgaagaacccatggtgatttcttctgaggtttgcatgttttaacttccttcatgcaaatgcttttagttactaagatttttattttatgttgtgtgtttttccatggagtatggtcattctgtgttttgagggcaaggcagtggggtaaagctgagttgaaaagcataatatggctcatcttttcttttcttttttttttttttttcattttttgctgcttttattttacgaaaaagctaattcaaatctacattaaactaagttgaatacaaagcctttgtgaagaaggcctggtagtctcatttacaaaaatggccagtgtcgtctttggccttaaaatttcaggacgggcacttcagatggcttcacatttgcatgtttcagtgctagagcatgggaatagactttggcatccactgtaagatgtttttcagctacgagagcatcgagtccctgcagcaggtcattcttgggtaacgaaatgacttccacaaattctccatcccctggctttggcttgggccttacattttctgcgtcatctccgttaatgattactgtcacgatgtgtgtggtacagtttgacaaacctgggtccatatagaccactggagaacattcagcagcgtcccctttgtagccagtttcttcctcaagctcccaaagagcagctgctcctggggtttcaccatcttctatgagttccctgcagggaactctatgcagtagccacccattggtggtcaggactgtttcaccagaacgatacactcgtagtgaagcgttctctgcagcacagggatgaccgctacatcatcaccagtctgttctttcctggttgtatgtttcactgagtcccaagttccagttttaccaataggagccatgtatgttgttttttcaagtttgacccattttccttctgaaattaactcctctgaaataatatagtgtttccattctgagacaattcagttggttcttggctctcccttttcaaactagtctttacagccctcaggagagaagttcacctccaaagtgtaggggtgacaaagggaagaggacaaaagaactactggaggcagcagtgtcagcatacgccggtgtcacgactcaagacatcggcacctcccgaggtgatgaaaggatgtgccatggctcatcttttcaagtcaactagtatttatcaagctgctctgtggaaggtgctggggacaggatggtacctgtccttaaaaccagaatgatcagactcagtggaaagagagttacaagagttacttgaacaactgtctctgattataaaataagatggagtgataaatacactagaatagagttatagataagctattgaaaaaaatccaagaagacttcatggcagagttcttgtttgaataggacttcaaagtgtaaataggaatagaaaaggcttggaaggacgggagaagggggaggtattctggtttatgagtacataaataagtacttggaagcttagaagtgcctggaatatctgatgtgagttgaagcattgagtgcattaaagaataataatttgagccagagtcaaactaggcttgattcttcaggcctgatattgtttcatgccagcagaggtgggagagagagggtttctcagctcattgatgtaatgagccactgtgactaatggaaattgcgtattcttcaggtaggttggggtgaagttcaggtagttgagaaattcacagctcagagagcctgtcttccttagagagagtataaaaattgactctcagccaggcacggtggctcacgcctgtaatcccagccctttgggaggccgaggcaggcggatcatgaggacaggagattgagaccattctggctaacatggtgaaaccccgtctctactaaaaatacaaaaaattagccgggtgtggtggcaggcgcctgtagtcccagctactctggaggctgaggcaggagaatggcgtgaacctgggaggtggaacttgcagtgagccgagatcgcgccactgcaccccagcctgggcgacagagcgagagactccgtctcaaaaaaaaaaaaaaaaaaaaaaacaactgactctcattgttaagtagtcagaaagaagtctcttaaacaagagtaccaatatcaaaagcttcattttagagattacttttgatgcatatattgtaaagccacttgtttgagatttgaagtagtcaagatttttcccatatatgatttactttatcttgtggtggctacccagttaaatatattcctgaagagtgttaatgtgaggaaactaaattttttgaattgagtcattttagatgagcaagaattttttttactgatttctattgtaatataattaatctgattattttagagcaagacagtatccatcccacccccattcctactctgtcccatgaacatatgctataagattttttacttgtcaacatcttgaactgagaagccagtattaaaatcttaacctttataacacaatgatttttgccatttgcagatgacaaaattccctgttattcttaatttagaaataatgtgagtggacttgtttctagtttgtttttgcttgctaaagaacacaagttgtttgggtttagggtggggtttggcgcaccttttctgaaagggccaggtagttaatattttaggcttgggaaccatatggtaactgtgcttgttgtaattggaaacacctatagactgtacataaatgaatgggcatggctgtgttctaataaaactttatatataaaacaggcagagcctggggtttggtgactccagcctagaattttcttccatttaagctgatttcatgtctgaaagaaagtaaaagacaggatgtcaaattactgcattaagttattcctataacgtaggcacctgtggcttatagagactgttgtttttaagtgctgctatgttgcctttatatgtataaccataagcaaaaatttctttcatcctgctctaccttttcctcaaatattttgactacctgtcatgtaccagatgttaggctggggataaatggtgaagaggcagagtggcccctgtcctcgtagagctgtagaccagtacagggggcagacagtaagcaaataaagtaattctaggctgtgataagcactatgatgttatcaagcatggtgctgagagagaattggggggtggggtgttatgtatatagagtgttgaggggaggcctcccttaggaggtgaagttgaagctgagacctgaagccaagaggatttagtcatgtgaaaaacctgatgggtcagaagacagtcatcctccccttctccctccctggaatgtcagtcagtataaacgtggataaactgttcacattttcagaggaaatattaaaagctattttataatatttttccccctctttttaacttcctttttaggtaagattaaaagcaatgagaacaaacaaatttttgtcttctggttaacacatgcacatacattctgagtatacattgaagtgttagctaccaggataaaagttatggcattgtggcctattatttagagcctttgggatgtgtgaagtttgttaagcattgttcatttgcattttataaggttgaaaccgcctgtatttgcgttctcatggcgctcctaaaggaacatgcgatgggagagcgccctctgctggcgactctccctcatagtgcctgctatagccagtgtgagaggcgaggcttccctttccagagggtgtgctccagtggtcaggctttggggtctcacaggccttccactgtcatatcacaagctctttaggtcacgcttgggagaatattcataaatagacttgtacatccctagctcctgtcttctgctgcttttttccttatctgtggccctctaattatcagtgcactttgctgaactggttactgacactgtgatggcagatgaagagggcagtgtaaactgagtgaagctcttgtgttttcacctgctgtctttggcagctaagaaccagctaaagttgcctcactttactgatacatgctgttgacagatgcataaaatgaatataaagtggatatgtgtttggtagcattttgaatctttgtacaggaggagcatgccctacgtgaagtgatcatcctgcaggagccttgacctagagggattcaccgtgttggctaggtttggggtgtttcatttttgttctcggtgtgttctgtttgtttgtttttgttttgtttgtcattgtttgtttttcatttttattttgtcccaacattttcctttatggtccaccattagatattgatcagttgaagctcattttaagactcgttggaaccccaggggctgagcttttgaagaaaatctcctcagagtctgtgagttgatgctttgtaattatctgccctgttgggagcctctgccacttgccttctaccaatctgtactttgacctgggtgtgtttgtaagcacacatgccctttgtgtgctgacttcccggatgagtgaggtataagacagtgtgagtgtgtgtgtgcgtgcacgcatgtgtgcctgcttgcatgcacaagtgtgttttattctctatctgggtacacactaagatcacgggagcctccattagacaatagtatcccagtaaagcaatgagatttgagattctaattcattccaagagattaatgtaaactttgtgtctctgtcctcaagacctgactttcttctttctcagaagtttcacgtatgctctagagctcttttcactgtttctagtgtttgttctcagagcatgagcttcaccatgtatttagtttaacttaatcactaaaagaataaaatgtacattgtctgcacactgttgccccttcccagaagttattttttattgttttagaatagatactcacatatcctatgtaaaatgtttgttcccaacagagggacctttctacccagtccctagaccccactcagaagaaacagcttcactcctgatacctcagatacacaggaatgtggttataaattttcctcctattgccttgtaattataagttagataacctgagacagagtttggaactagtgctttcttgcttctaagcagggacttagacagaagccatgggaatgtgagtctggcttacttttgctatgatttcctgctcttggtcattttcttgggccagaggcatgagagaagccatgaaagcccagcttttctttttccctcgatctgctgccaggcagttgaacaaaagaaaaaaagagctatggagccaagctccagttatgccttcctggattctcctcccaacatttatgactgaaggggccctttgcctagttgggaagatggctgggtagcttgaggcaagcataagcaggtgagttggaaccgttcatcatcttcctgacaaaataaagaataccgtcatctggtatgtatccggtacatttcttgaaaggatcccaaagcccagtgtatttctgaacctccactctgaatttgtttcatttacttatttaagataaagaagcaaaagcccagaagggcagtgacttttccatgtcatgcactgaatcacgtggcagagctgagtatagatcccagatgttctgataacatgatccaaactataatcccatgtgattacacactattttgaaagtcattgtcagtgttaccacaatgtcaacctaaagaggtatttttttctcctttatgatatgtagaagctgaattagagtgaatcaaagatatcagatgatgcttagaaagagcagtactttttttttaatgtttttgcttagaaacactgtcttacagtaagaggacctaagggcagagggaggagctcaattaataaatagtgctaatgagaccaagcaaattcatttccttcctgagagtgtctcctagcagccctttcttagtcttctcttccatcccaaccatctgccctatagatacgtgctattagtcatttggaattggattaggaattcaaaccaaagtctaggtaaaatagtcaatagtatttatctgtcattaatccatcacttatttttaagaaagaaacaatgagtgcacagcctaataatggaggtctctctgtgaaatgaggcacattatacatttcaacagctctactgaaattgagtaccagaatacagaacttaactcaaataatattatattgagtataagaataagaaaggtttttttttttggtttgtttcctacataattggggtgagtggataaaagtagtttaaacatcctaaggggtagacaagattaatttgatgttcgtgacacggcccaagtacctgtttatcttaaaagcgcatgatgtatgggaacagaatggaaaaataaggccaggtgagtaaaattgttcagagattttaaaaattaaaaaaagaaaaaagacattagtgaaggagtgcagtaaatagccaacagctgttttcattctcctgaaaagtagtgaagtagtgatggcaataattatgaagaaattgaatgccgagtaagagaatgcagtaatggatttagaacctattagaatgagccataggaaaaggtataaatgggttgtgatggaggccaaggacagagaattcatttggttaatgtcccagtatctataataatcagtgaaagaacaagaaaatcatattaagaaagcaaaagcagactgggcacagtggctcacatttgtcatcacagcactttgggaggctgaggcaggaggatcactcgaggccaggagtttgaggctgcagggagtggtgatcatgcactgcactccagcctgggcaacagagcaagaccttgtctttaaaaataaataaactaattaattaaattagataaaaaagcgaagacctgactcattcacattttcctccagaaatagaatctttctaacttcttgacagaaaggggatataataacctctttgactaaaggagacaaagacagaaaatgtgcaggccaggtatcattcacagaaagaaaggggaagtggaggatttttacattttaagacccaactggtattctgctattgctggtttactttgtggcccctttttgttatttcccttggcatgaaaagtatcatttgttatgtgatttgttaaaggtaactctaaattctgttatctgtactcctaacaaaataaagaattccatcatctggtatggatctgttaacgtttcttaaaaggaccgcaaatcaaagtgtatttcctatccccttttggaatttactggcctctgtctttgggcttagggagcgtaaaaggcagcgtggagcacatggctctagctccctccctgtggccagccttaaacagattactgctaagtcatctgaccactagggagttcagttcagaggacgagctaaagtagaaaaattatttgggaaagccatgaagtaaaattgcttggccatggtgcttattaaagatgacacttcacctgaatgagggaattagtagttaacaagaacaggactagaaaaggggtttattgaattacattgaatatttttaaaatctcatcacttctgctcttagtaggaacatgataaacagttctgacattttagaagagggtgagttgtattaacattgatggaacagttaaaagagagaagttgtagtacagcttacatatgtgcctatacatatagaaaatctcatagttctgcccttatcagatgcttgcagagtattctgcagacttccctccagctgtttgttaaatgaggttagtctgttcgctagttgaagagacttgtggaggaaatcatgtggcatgctttaacactctcagcagcctgtgacttctgttttgcctttgccattagcctctaaccttctcctcttctcctctttcttcatttaccattttgtgtttgtggcaagtattttaaatgtaccactaactaatgtctttgaaatgtggcattgcttattaacccagatgcccacgtgtgtcttaattcggagggatggtgccttggagaacctgtgccgcaaaagtttagtctcttccttgaatggatatgttggatattttattttgttcaaagttgagtcaaaatttttggaatcatctatatttctcccacaaacaggttttggccatcagtacttgaaaacagttctatggctggcatcctggatcaaggggcagtctgtttccagggaacctaaaatatttaacagtaaagatgatgctaaaaccaaagaaaagtaatttttcttatctgcatggccagtcactggctacgtgcctagttactcatgtcattcacagaagtgtactgtagcatgcagctgtgggctctcgggtagggggaatggagggtaggtttttttgtctgtttgggggtggctttttgacctttttaaaaacttgagttgaaaattattttttgcactgtatgtttaacaatgcccttgactaggcatctaagatattaaccagcttcagcagattatgcgtctgacaggaacaccccccgcttatctcattaacaggatgccaagccatgaggtgagaacaaagagactgtacagggatgtcgcactgagaagccacattctcattttattgccaccgtataaccaacagttcttaacatcactggatgcttgcatgtgccttgaggcctgcctatgtgctattgcaagatgttgacttttgggataaaatgtcatccagtcctgtgagattgggaatataattagtaagcttgtaaatggaaaattgttatttttatatttggttgtgtgaatttagtgcttgtgaggcggtttctttctaggtgctctgcctgtagctagggtgtgctactatcgtgacctttatcagaagggtagttaactgaagtccattagcatgctttccaattcatccatttttttaatgaccaaaagaggattctttatttaaaaacaaaactgaaatatccaatactaatatgtactatatttaaaagaaaatacacaaataatatagaataaatacaaacactttggtacagttaggtaccttctttattaattattaactacttagtctaactcttgttttattatcagagatgattggggcacttctttctttgtaagatacccttaatattttggggaaccaggttggcatcacagagggcccttcccagcagctttgtttctacttcagctatattcaggtcgtcttttgatttatctttattaaagggatgactgttctctgaatgagatagttctctcttacctgttttcccttcctctttttatttacatgttcaagtagaaagggattacagccatttatgtattcaattcctaaaactttcttttcaaatcagaaagattctcaattgcttttcaaagcagacctttcctcctgactccacatgcaataattaagaagcggaaggctgggtgtggtggcgtgcacctgcaacctcagcactttggaaggctgagatgggaggatcagttgagcccaggagtttgagaccatcctgggcaacacagcaatactgcagctctacaaaaaaatttaaaaattagggcatgatagcacatgcctgtagtcccagctacttgggaagctgaggcaggagggttgcttgagtcaggagttagaggccatagtgagctgtgattgtgccagtgacctccagcctaggcaacagagcagagactgtgtctctaaaaaaatttttttaataaataaagaaggtgaacttgcccaagcaaccagatttttttttttttaagacaggatcttaactctgtcaccccagctggagtgcagtggcacgatcttagctcactggaacctccacctcctgggctcaagcttcctgggctccatctccctgctcaagcgatcctgttacctcagtctcccaagtagctgggaccacaggcatgcgtcaccacacctggctaattttttgtatttttggagacaagttttcaccatgttacccaggctggtctcaaagtcctgggctcaagcgatctgcccaccttggcctcccaaagtgctgggattacaggcgtgagtcaccgtgcctgggcaaccagaactttttataaacttgtgtgtgtatgtgtaaacataaatatacatacatacttttactgttggactgtattaaaacaggagttttaaagaggtggttaagatagacagcagactttgcttctgaaggtgatagtgggatagcagataatgtttcttgaactgtcacttggaaccaagtactgtgttaagaaagtactaggtttgttatctctaatcttcactccaaccctgtgaggtaggactgttatattaagtttatattaacttttttgttaccaccgttactttgttagcaaggtttaatattgatatcctttgatgtactaagagcacctactgaagtcagcactgtcaaaagactgctggtccaagacttgctttgggaataccaacccctctaccaatttagctgtttgagaaggctgtctcagggctacttcttacatatcttggcctcagtttttaaacttctcatcctgtgatcagaaagctaatgaaagagaatttgattgatgagcattctcgttcacttgaaatgcaactgtgagagatagcaggaataaagaaggtgatgatcataactagggaagttaggatggcagtaagggagaagttctatcaaagacagttagaaacactgaagttagtggactttgtcagttaacactcgttccttagcaggaccaagattctttctttgagagcagtaacattattgaacaaagtcattctgaaaacccttgttttttcaggcaagaaactatattcagtctttgactcagatgccgaagatgaactttgcgaatgtatttattggtgccaatcccctgggtaagttgaccatatatcctcacctcatggatattgaattggttatgatataaattggggatttgaagaagagtttctccttttgaccaaataaagtaccattagttgaatcttggaaggtgataaatacggcttttattatctatttgtgttgtcagatttttttttaatcacatgagatgattgtatgtttaaatgatcacagaacttgggattgggggaatgggcaggtacagtggagaaaggagagaaagtaatgggaatgttgagagaaaagattggtgaggcaggaagaagcagatacagaagaaagctgtaacttgctcacaactgctgtggatttatggtgtctctgagtggaggacagaaaccactgaagtgcaggtgggtgtttagaggagccacccaggagctccctgcagggtgctctgctctaacggtagctgccgttgctacagatcctgtcgtcttccacactcatgaacttttcccctctcctcttctttctctgccctcagaggcataaccgtcctctcctgtttgtggagcttatttgctacctaaacttaaaggaatggcttcctatgccttcaagatccttggcagagggaagtgatggtggcagctgaagatatgtgttgccttggctaaaaaaggagttgctttattattattatttatttatttatttatttatttattttggtagtgtcagggtctcgctcttttacccaggctagagtacagtggcacaatcatagctcactgcaagcttgaactcctgggctccagtgatcctcctgcctcagccttccaaatagctaggactacaggcatgcatccccaagcctggctaattttttaaagaattctttgtagagacagggtctcaccatcttactaatcgtggactcctggactcatgcagtctgcccacttcaacttccaaaagtgctgggattcagacgtgagacactgtgcctggcggactatattctttgaaggggaggagtgatgtgtgcttcagatggcccctgaaagctgctttgcctgaggctcattccctgactcttgaagaggacagtggacagtgctgtttaatggtggacacagaaggatcagaccaggacctccctacaatagggaagggagctatgctatggtaacgagtataatctgccttctcacttttcttaagattgtattgtgtggtgtgttttaaaatctcactgcatttctttgcatctgtttgggtcatctgtcttcttaagggtcttggcagctatgcttcaggtttagttttatctttttgagggctgacagccatgctgacctctttagaagctggggtgaaggctaggtgattttacctttttaagtacttcatccatagtgattatggagaggaattttagtgtcgctgtgataattcttataatagcagagatactcacgctctttgaagttttgggcagttgtggatgagttcgtgtcagccttggagacagagagtatgtcatatctcagctgggtcgctctcctgccctaggatgaatccacagtctcatcaggggtgggtacgattctactgagactggcttaggtttgcatctgccctggcatgctctcattggctttgtgtgatttatttgagctgtattatctagttcaagggtctgtgagaagctctctttgagccttaagtactacaggatgttgggaaaagatgttgtgttctttctttccttttttttttgagacagagtctcgctctgtcgtccaggttggagtgcagtggtgcgatctcggctcattgcaagctccacctcccggctttaagtgattcttctgcctcagcctcctgagtagctgggaatacaagtgtgtgccaccacacctggctaattttttgtatctttagtaaagacggggtttcaccgtgttagccaggatggtctcgatctcctgacctcgtgacctgcccgcctcggcctcccaaagtgctgggattacaggcatgagccactgcacccagcccgtcgtgttctttcttgtcgtcattctgttctgaagttgtttcattttataagccttaagttttgaacagggcagtactcactgacaccctcttaacagactcagaaagatgtgttaattcattttttagggggagaggggagatatttgtgttccagcagcttatttaggtcacatacctcctgataggtcttcccattctttgaaatcatgacattgaaacctccaaaagaactagataccactttgctaatgagttgaaaggaggctttggtccatctggtgctcagcactgtacttctcagcatttaaacgtgccttacagcccattgccaaacagtgatgtcatataatgaggctccccttgctgggcagatggagcacagtgttactatcacaagctcgtgcccagcatattaactgagcaggaacatgccagaaatataaatattaacagctctgttttgtcagccactgactttgtttcactgctattcccaaaacatgaattaaaacttaaattaccccttgatgttagtgtatgtatttatctgtcaagattttttttttttaagagatggcatctcactctgtcacccaggttagagtacagtggcacagtcacggttcatgataacctcgaactcttgtgtttaagtgatccttcctcttccctatcccaagtagctgagacaacaggcacataatgccatgcccagctaataagatctttgaggaatttgttcttgcccctgaccttattggttataagaacatggtcctttttaccctaagggtaacagagacttgtcgtataatctgtgttcaattataaaatcaacagctgataaggtttctttttctaagaagagaagaccatcagaagaatatatatatatataatccaggcattcacctagtactgtaaagtgtaaacttatgaacttgaattgacacatgaatatttactttgtttctgtatccccttgaagcccatatttctccagtggattactctggaattcaagctgtaggacgggtgttttacagcctccaatatacacagcagtagatctctataggaagctgtcctcttcactactcacccccacaccctccacatttaaggagtaatttgcaaagtctcaaaagcctttttcaccacagtgataactggttatatggcagtatttattctcccaagtgccaccatgctgaaacacaaaaatactcatttgaaatatagagttagacaaattaccattttgtaaccccttatgaaataattgcttttgggaaggaaacccagtgaatgcagaaagccataagtgaaaagaatggatatttacttggtaccaaaatatcccgcagattacttgctttggtcaaaggggaaacatcttaacccagtgatcaattttagctcattagtagtgggacagatggccttaatgcactgtcacgtgcacagcatcacctgtatttttgctgaaaatgtttaacctgattcttaccaaaatttttgacctaacttccggtttttgggaagtacaggggatagtggaaagcagggtatagacacatgacagcaaaagaagcaaaatcagacacatcccgagaaacggtcattctcaggacaatcagagggtctctttagatcaaaatcaggaaagaacagggcttagaagtaagaagattattctagactaagagagactaaagaaaaccagtcagttgcagtgcatggcccctaattgaatcctggtttcaacaaactagttctaaaagacaacgttggaataattggagaaatcttaacatgaactggattttagatgatatttagaaattatttgtcagagataataacagtattatgattatataggaagatgtcttcgtgttgaaatatttggggagggtaaagtgtcatgtctataattttattttaaaacagttcagaaaaaaatacatataaatacagaaatataaaaatgttaattgttgaatatgctaggcatatgagtgttcactttatcatagtttttacttttctgtttctgtatatttgaaatttttcatagtgaaatttttaaaaataaaattccctttgattttaataccatgtctcaccctcatcaccatgtaaaacccccagtttattcactgtttgaagatctgttgatgaatatttgatgttagtaaataaacagcttagggggacaaaaaaaaaacagccctgcctacctattgtaggttacttcagctgtaggctttgttttatacagtttgactacactggtagcagtttgacattgacggtccacaaaagcctgttcaaacagaatcccagtcatttaaaaaaattcttctttagtaacatattctttaaaaatacaaaatacaggaggagggtgagggttgagagataacctatcgggtacaatgttcactatttggttaatgggcacgctagaagcccagtccccatcagtccgtggtgagacccatgtaacaaatgtgcacatgtacccccaaatcaaaaataaaataaaaaaattttaaaacatagaaaatacagatacacacacacacacacacacacatacacacataaaggagaagagggctaatatatcctagagtaggtattttggaggagagttctttgtttggatatgaagggtcaaaactatgtttgctcaataaggcatacttttttgtaacatgttaaaaactcttttccttcctgtctatggtactgatagctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagtaagtcctaagggtaggattaacatcttgaaccactaaccaaaagcggtgggaaaaataaaaactgaatggtcataaccaacttgctaaagcttgtagatgaggtctctaatgcagaatgaatatgttctctggtggaaactgttgtagacagtgatactctctggacctttgagatacttatgtctggtctgattttatgcacagcccctcacataggagttttgcatggagagttgaggttttcagagtcattgcatttacaacatctctctgagcttttacagttttacaattagtagaactggggcaaagagaatttatgtcttatcagttaacttagaactagttagggacatagccaggaatggaacccatgtatcctgtgtccagtcccatattctttccacaaaacacaagttcagaactctagttaaatcccaagagccaagaccatctgttgctgtagcttgtgagagtgctctttcattcttgagcacctccgtagagagcttaaaagaagaggcactcagcgatgaaaaacatgagaaaaaaaatggttcttttctatgtcccttgaacttctgtgtgataacgtgttccagtaggctattacatacaagctgtgaggtagcccatcaaaccactacctggacagagaggaaggatcttgagcttagaagtcagagtgcttgccagcaaagagaatagcctaaactctcacatcttacttttccttcccaatttctaggcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacagagccgcactcatctgtttatgtattgtctgtggctgcttttgtgctacagtgacagaggcaaatagttgcagcagagactataggacctccaaaatgtcaaccatttaccatctggccctttagagaagtctgctgacccttggcccagggctacagttttctcatctataaagtgaagttcaggtttttttcatctttgacatttgagactatctataagccatatatttttattataaaactgccagagagaaggtttggtttgtatatataggtgtacattgtttgtaagcttttaggcgaatgtattttatgactctgttttgtcttagctggctacggtccttgaaaatattcttcagaatgactgaattgcatcacgggatgaaagagccacggggtctcacagctgagttctcagggactttgctgatgtgcttcctttcttggtgtccttggaatgacacccagcaggtgtcccagggtggattgtggggctcagcctgtgaggatattgagagctgggtttttgtccaggccctggctggcagctgggaatgaaggcagtgatcgaaggcagaccaagcatagtctcctttccatccccagtgacttcgtaaggaggtgagagaaaagggccccggttgttacatggggtcctttagctcctgttaagaatcagacagttggccgggcgtggtggctcacgcctgtaatctcagcactttgggaggctgaggcgggcagatcacgaggtcaggagatcgagaccatcctggctaacacggtgaaaccccgtctctactaaaaatacaaaaaattagctgggcgtggtggtgcatgcctgtaatcccagctattcaggaggctgaggcgggagaatggtatgaaccagggaggcggaacttgcagtgagccaagatcatgccactgcactccagcctgggtgacagagtgagactccgtctcaaaaaaaaaaaaagaatcagaccgttgtagggagtattttgccttcacataccagacatgagacatgagaacagccttcctcctaggcttctgagaagagggcctgcttagcccaaagagagctgacaccacaaaaagctcccctgaagtgaatccacttaaaacggggaccaaaatttcatatgtggtggacactgggggataggggaaacctgaatggaactttaatcatgaaaatggaacaaaatgacaaaagcatgttcataattttaaagatatattaattctgacaaggaaattaagttgtttttgaagtgaaactgattttgtatcttacaataattttctcatttgatttttgcaacgtgatcatagaaggaagttttgcaaaaatatttgagggcttcatcagttttcagaaggcctcttgatattttgatattcacattcttttttataagtatctatcggtgatcacctcctcaactttccttttattgttaactggtctgagtctgtcacccacacttttcagagcttttgcttaaggtttgagcagttttctagcattatttatcaacttcatcaaatccaccaccatttgcaagatttgtgattattgcaattaagttgcattctatttgtgtcatttgtcatatgcttacagtataaccatagagactaacagaagaccagaatacagtgggcaggataaatacattggcttcaggtacttaagaagcaaagagggactaactttttataaatagctcattagtgaatgttttcacattgggatgactttcacttctctatataatttggtagctgcaaggacttggttaatgaatatgcaaataataaagacccccttttatttataatgcagatatgttttaataatgtacatgccctttgaacttagcatttacacttctcagaagtcttcagcctgagcaacatagctggggactcttgactctacattagccaggtgtggtggtgcatgccttgtagtcccagctgcttgggaggctgaggtgggaggatctcttgagcccaggagttcaaggctgcagtgagtcatggttgtgccactgcattccagcctgggtgacatagtgagaccctgtctcataaaaaaaaaaaaaaaaaaaaaaaaaggtcttaagtgcacaaagtagaatgtttttcagtgtcacattctttgtacagcaaaatactggaagtaacccacatatctatgaataaggacttgattgaataagatggtcatctgtgcagtggaataccatccagctaggcatggatagatgtgcaacatacattgtgaaggagaaaaagtcagtggcaaactgtagtatcctagcccccttttttgttgataggtaatgcctggctaggaaatgaatatacattgaactgttagcaaccatctctaggcagtgggattgtgccctttaatttttatgctatttctgtaatgtgtacctttttttacactgagagttttgttttgtgatcagaagaaaccttttctttctttgttttgttttttaatgagaaaggtgtttaaaagtctatgtgacctggggaaccattctcaaatttcagttttgccagggctagagcccagcctttcaaggcaggagaacagttgctggaaagaaattgcacccagctgttaggcctctggcaagcaaagccgcaccagtactttgtagcggcattctccacccagctgggagggtttggttgtttcttcttcagtagaagggtgtattttaaggatcttaagcaggatttttcaattttggcactattggcatctcagaccagataattatttgttgtggaagctgtcctgtgcagtgtagggtgttaggcagcatccctggcctctacccactaatgccagtagcactcatgctcccttcccccagttgggatagccagagatgtctccagatgttgttgccaaatggcttctggggaacaaaatcatccctggttgaaaaccatgaatctaacacttcatccctcctaatcttgcagaacaccaaagcctcttttcttgaaaaaagagttaacacccactggcctgttgtaacttaaagaagtagtttggtggccgggcacggtggctcatgcctgtaatcccagcactttgggaggccgaggtgggcggatcacctgaggtcaggagttcaaaaccagcctggccaacatggcaaaaccctgtctctactaaaaatacaaaaaattagccgggcttggtggtgggcacctgtaatcccagctacttgggaggctgaggcaggagaatcgcttgaacctgggaggcggaggttgcagtgagccaagatgacaccgcagcactccagcctgggcaacaagagtggaactctgtttaaaaaaaaaaaaaaaaagtagcttggcatcttggaggaaaagtacaaattggaaaggcctttttagactcaaatacatatttttaggggaaatatgtttggtactgaataaaggtcatttcatttcttaaaaaaaaacagtaggttgagtaaattaagttatcctttgttcagaagttctttgccattatccacaggcagaaaatctggtaattaaactgggggtatgactacaagattttcttagaccagtaaggaaataacaaatctcaatgtgcctggtgtgttgtcgacactcaagatactggttagtctgcatctcacgagttgacagggggcatcgaggctgtcttgttagggggagtttgcagccttctgcatctgggaacagtgtgtgtctctaaagccaactggggagagagcttctgaggacaaatggtcctgaggcagtttctggaaaacccattattgaagccataggctgtggtctaagtacgcatagccagtgggataatgttatattgcagagttatttgagtagttcaatttaaggaaggtgctttgtttagaagttattataagttaaataaggaagtaaccttcagtttgcaactaaaaatagaaactgccaccaccatttgagtgtcagttgcagcagaaaagaagaaggtgtaaaaccacagacccagcctagattcttaacaaaaaccagtggttctaaactcttgtcagatctattaccctttgcataactaatattttataatacctcttttataatgctgaggtgaaattcatagataattcacacaatttcaaagaaaatataatgatccaatataaatgacaaaggaaaaatacgaatacatttccatatataagtgctggctgggcatggtggctcgcacctgtaatcccggcactttgggaggctgaggcgggcagaacacttgaggtcaggagtttaggatgagcctggccaacatggagaaacccgtctctacagaaaatttttaaaacgttaactggggcggggcgcagtggcttacacctgtaatcacagcactttgggaggctgaggcaagtggatcactcgccatcctggccaacatggtgaaaccccatctctactaaaaatacgaaattagtacaagatagaaaaattagtcagctgtggtggcacccacctgggtcccagctactcaggaggctgaggcgggaaaattgcttgaacctgggaggtggatcttgcagtaagctgagatcatgccactgcactccagcctgggcaacagagggagactccgtctcaaaaaaaaaaaaaaaaatgaaaaaataaaaagttatctgggcatggtggcacacacctgtagtcccagctactgaggaggctgaggcaggaagattgcttaagccatggaggttgaggctgcagtgagccatgatcattccactgcactccagcctgggtgacagagtgagaccctgtctaaaaaaaaataaaaagtaaattctcaggcatagctacataaaagaaataatgaagtagttagatgcttgtacctatacatagaatcaccttgaatatgacagctccaaatgcgtactgatacagttgtgttgtcttgacaactcagataccatggctgactgctgtcagtcatgatttttctgaaagtggtaagcaactcttggtaaaaatttcaaacaaacaattagaatctttttttttttttggacagggtctcagtctgttgcccagcctggagaacttttttatttactcagttgtgatcctgggaaatgtaagtatattaatatgcagaaaagtatttgtttgatatgtaaaaccaagttacagtctaggctcagataatcataaatagttttttccaccttcatgaatgtccagcaggatgtttgtaatgtgaagcaattcttgttaagaatcccactctgcacagtggcccgtgcatgctcatatcagtaatactcctgatcagtatgataaccaaaaatggccccagagatttcagtgcacccgctgagaactgctgacagatcaaatgactctgggacacctggagtcctaagaagtgaaacctccacaatgcaggacagacctggctccctatggtcttccttccttccttcacctccttccccctactttccttacctccctactaaaaagtagttcttttaatgaactgctactgtatgttaggcaatttacattcttgcatattctatacaagtaatgtctctttctcagtcacgttacaactgaggctagattcaacagactatcctgatatcaacgttgaaagactcctggttccaaagagttttatagcattggtgttagacaatcctcttttggtttttttaaagaaaatttaaaaaaattaattcagcataatttccataaggactccctggctgctgctggccaaattggggaggagtccctggcagccacagccaggcactggtgccttatcttgttcagcacacactgggtctctctccctagttccaggccccttgccctcgcttctctactcatctgggatagaaatgcacctccaggcctctgaagagaaactaccttttccctctttcagccaaaagggctcaagccagcactcgctttgtatgtcctagaagatgagcatgcttccctctggctctctatagcagtgctctaatgggacaaagtcactgtgtgctcttgctttctctcctggtctttgtgcgtgatagtccctctctctagaaccctcttcctcctcctcattcctctgagctaatgtattcgttcttctgttcagccttgacatcactccctcagtaaatttctgaccattcccaagcctggattagctgtgtcttctgcatgcaccctgtaatctggaatgatcattggtggcactgattacactgctttatcattgcttgctgtttgtaccacctgctcaaaaaagaaaaacatataaaggttaaggatacaggctctggaacctttgatgtgattaagtaatttgctcaagattgtaccaataatagagagtggggctgtgaggactaaatgagttaagacatgaaaaaccctgagtatacagtaaatgctaaatactggcttaaggctgcatgtcatcgaagacagatcatgtcttgttcactgtggtgtccctgtcacctagcttggtacctggcacacagtaggcattcagtccttattgaccgagtccataaatgactgtcagcttttaatcccttgcagatcaccttgtcttttgcttcctgtttttacaatcaatagtctttagaagcaaagatcatttaaagaaaaaccaaatatgtaggggttatctgccttgcctaagaaatggtgctgtgtggaatttccttgtttaagaagagatacgtgttagaagaattggcttctgattctctttgctgtgtcttgctgtgtaattcaagttggctgcacagcaatttaatgaattagaacaagaaccaaaagattattgaaagtggctgatggccataatctagataattcctactgtgtccccatccatctgccactttaaaatgaaaatagcaaggcaaaagaaagataattggaaggcattatggtgtcttgcaccctcattcagccattctacaaatatctattaggtaccttccatctgtcaggcactgggaatatgggaaagaataaaagtcaaattcctgtccccatggagttgttcgttgttttcatgggaaaaggcagtggtaaacaatgactgtaccatctcgctggcacccctagaacagaacatctaagcacctgttgctggaggccatttctgggccggtgcacctttgatccacctccctggcactcaactgacagacaacaggatttctcgtggcgtccagggctgcatgtcctcttgggttacctcgacctgatcagctggctccactgtgtgtttccagtgtgaggtgtgaggtcaccatccgctgccattacacaaagacccatttcccctgggccattgctcaagtatgacagagctgattgtcctgtatggaaactcagccactcacagctctgcccagtgagacaggcaatgactatctgatcatggttgctgacacagtgcttgtggtggggagtgtccctatctggaggcaaacagccgagtggctgctgtgccctcaacaagccccaccctgcttcacggtgaggaaacagcccccacccaaccagtcctctccccagcctccagttgagtcaggtgcccccctccaagtgtttatgtaatttgtgctgatcttaccagggtcaaagcacttaccacattactttgtctaactattattctttcatgacacaaaacatatttattgttgaaaatgtttaaatgtagtaggtaacatctattatcccactacctggaaataatagctgttgacagattggcataactccaagaaatttttttaaatctggaaaacatcgcttttaaaactcattattattcagtgatctgtgtcaataaatggcattgaaacagctagatagacactttcctccttcagccacaatcatttccagacaggtcaaagatttaaatggagaaaggctttcaaagcatgtccccaaacctagaaaccatgcaggaaaaggcaaatgaatttgcccgtgcaaaaactgtagagctttcatacattggcgtccatttggcaggattcagtaagggcagaagctgtacatagctgcggttctaggtaattctagaactttctagaagctttctcgtatgtcaacaaggaacagagtacagacttttcactcaacatcatttgtaatagcaggaaaaaatgggaacaaacctcaaacaccatcaataggggaataggaaataaactgcagtatgtattcatgcaatgaaatattaaatggcagtgaagacaaatgaactagaatgattcccccaccaccaccccagacccctgattttgcctctgtcccaggaacaataggcaatgtctggagacctttttagttgtcacacttggaggtgagggtggtgagcatctagtgggtagaggccagggatgctgctgaacattttgcgacgcacaggacggcccccacaacgaggaattacccagcagaaacatgtcagcagtgccgaggctgagaaacagagacgactcaacatcttggcagatctcaccaacaaccttaagtgaaaaaggaggctgcaaaacgatctatctggcatcgtttacagatgcatacgtatatagtaaaactaaaagcagacgagaataataaacaccaacttgtagaggggtcacctctagggagggagagagggcatggaatcagagaggggcatgaaagggcttcagctgccaagagccagacagcttaggttctaatcccggttccaccactgacactgtgtaacctccgcaggttgcttgatcaatcggggcctcagttatcacaactgtaaaatgggatgatattgataatagtgccttcttcacaggattgtggtgagggttaaatacgttaaccaatgtagagcacttagaacagtgtgtggctcatagtaagaactatatgtttttgtcaccgttttcatcatgatgatgagtatctatgatgttctatttttaaaatatcagaagtgaatatggcatgtgtgtgaaacatctcagcgttctgccttgacaaggaccttcactcctcctcccacccccaccagccaagtgaatttcaccctcagcacttagtctatatttgttaaggctggccatccggaacatactctccaaatctgagacaattcatccaagcagtgtgtttccttttcctcttcccccaccaccaccgccaccccccaccatgatgctggaaacttttctttattgatacagatgatgagcaatgcggtgataagtgtgtatcccccggctgttttcgcgttttcactaaattgatggctatgggattaccggattaacaaggtgcacattttgacactttggcagcattgccaaatggttctaaaagcttgccccagttgacactcccacctgccgacgacagtgttgtcactgtttcctgttgccgccctcactagggtaggagcttctcaagcacaggaactgtgacatttgtccgtgtattctcaatgtcttgagaggtgcctggcaaaatttggttgaatggattaaggctttaccttaatcaccaggtaggtaaggaggatagagaaaaataagacactcgtgccctcaagaacttacaatttaggtttgtttgaaagttaactgagaattccaagtctaagggtgctggtgagagtggcctggcaaagccagcccaggaagagctgctgagcaggttgtaaggaacgaggatgccccaccccccctccttggcaaagcagaggatggtattccagacaggtcacaaacagctcaagcaaagacgtggtgacagggatgaggaaggcacgcttgcggatcgctagaatggaggttgcctgggcacagacaccttggaggatccgattagcaagaaaggcagggcctgtccggatgggggcaagccttgcccaacagactaagaagtttaatctttatcctagaggccactgagagctaccgggggtggggcgttatggggaggcagggctgctctgaaacaggaagattaatcacagctgggctgcctgccgctggctgcctgtcacatgggagctgtgctgccctcaagctgcccagctggagcaccctctttccaggtgaccaaagctggatccgggtgccccctggcttaggaagggcagccctgggcaagtgtgagggagtgaagaccaggcctgagaagcagggagaagctgtttgtcagggaagtgtagtcaccaggatcagggtggcttcccctccacagccccttactcatccctgttgccacacctgtgctcgggctgacagtgacagtggtgtcctggagctctctgtgactcttatttttttagagacagggtcttgcaatgttctttctacttaattttatttcttacaaaagattggcctttcacaagcatcatccagcgcagctaagcatagtggttactagcactgaccctgggcaagactacctggtcttcactcccagctctagtgcttactagctgtgtgtccttgggcaagtcacttcacctctctgtgtctcattgttatacataaaatgggggatactgttaacccatctcatagagtcctcaggaggattaagttacatgtatgtaaagctcttataacaacttttgggaagtaattagtgctctttccatgtttattaaatgcatataatagaaaatgaatgtatccagagaggcagtctgacttacctaaggtcacacagcaaattagagacagagctagaatcagagcccaaacctctgccatggcaggtagccctgctagtcttgtttggggttatttgcccctgaccaaggtccatagggcttctggggaggatctatgtgtcctgacattggatagtttacttagaaggggtggagcaaggagaccctgtggcctctgccctggacaggcaggttgtggaggtggcctccagggtgcacagcttctcctgcctgaggcaggctgcacacaggagtccaggcaggacctgactccattactgcggtttgtcaggtcagcacagcagagactgccccaggcacgaaggtgagttgggcctgcatatcagctgcacacagacagagatgcagcgataacaaggctgctggggataaggccctgctacatccctgccattcggccagtcaccggctgccaggcactcttccaggtacgtggaattcagtgatgaacaaaatagacaaagcccctaccctcgccaacctcaagacattctattagaggggggcagacaataaacaggtacacagggcttgaagaacacgaggccaggtaaggcaggtgagcatggcttgtaatgaggtcagggggacctcggaggaggtgacacatgagcagtcctgaaggaccagagggagcaccatgcacaatgcctgcaacctggcatgtgttgcttcagtaaaccctcctggtgtccctggctatgcaagtgacattctcccatcttactgatgaggaagctccacctgtagctacaaatggaggcacgagattcagactcagccctgtctcccagaactcagctacctctctactggctcacaggagtacaagcacgcttcaagaatgtggacgtcacaaagcctgtggagggg

SEQ ID NO: 3 - DNA, 4353 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattaaccagcttcagcagattatgcgtctgacaggaacaccccccgcttatctcattaacaggatgccaagccatgaggcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 4 - DNA, 4353 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 5 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 6 - DNA, 1431 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctgggtaagttgaccatatatcctcacctcatggatattgaattggttatgatataaattggggatttgaagaagagtttctccttttgaccaaataaagtaccattagttga

SEQ ID NO: 7 - protein, 297 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLGKLTIYPHLMDIELVMI

SEQ ID NO: 8 - DNA, 4274 bp, Homo sapiens
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 9 - protein, 307 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESLSTCWRRCLYWTQIRELQRPKPLHMPTLLSTTILMMNQWPILMISPLKAGTSL

(19) United States
(12) Patent Application Publication
(10) Pub. No.: US 2020/0316122 A1
(43) Pub. Date: Oct. 8, 2020

(54) METHODS OF PRODUCING T CELL POPULATIONS USING P38 MAPK INHIBITORS

(71) Applicant: The United States of America,as represented by the Secretary,Department of Health and Human Services, Bethesda, MD (US)

(72) Inventors: Shashankkumar J. Patel, Clarksburg, MD (US); Nicholas P. Restifo, Chevy Chase, MD (US)

(73) Assignee: The United States of America,as represented by the Secretary,Department of Health and Human Services, Bethesda, MD (US)

(21) Appl. No.: 16/754,926

(22) PCT Filed: Oct. 10, 2018
(86) PCT No.: PCT/US2018/055206

§ 371 Date: Apr. 9, 2020

Related U.S. Application Data
(60) Provisional application No. 62/570,708, filed on Oct. 11, 2017.

(51) Int. Cl. A61K 35/17; C12N 5/0783; A61P 35/00

(52) U.S. Cl. CPC A61K 35/17; A61P 35/00; C12N 5/0636

(57) ABSTRACT

Provided are methods of producing an isolated population of T cells for adoptive cell therapy, the method comprising culturing isolated T cells in vitro in the presence of a p38 mitogen activated protein kinase (p38 MAPK) inhibitor, wherein the T cells have antigenic specificity for a cancer antigen. Also provided are related isolated populations of T cells, pharmaceutical compositions, and methods of treating or preventing cancer in a mammal.

### CROSS-REFERENCE TO RELATED APPLICATION

This patent application claims the benefit of copending U.S. Provisional Patent Application No. 62/570,708, filed Oct. 11, 2017, which is incorporated by reference in its entirety herein.

### STATEMENT REGARDING FEDERALLY SPONSORED RESEARCH AND DEVELOPMENT

This invention was made with Government support under project number Z01 BC010763-12 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 155,065 Byte ASCII (Text) file named “740369_ST25.txt,” dated Oct. 5, 2018.

### BACKGROUND OF THE INVENTION

Adoptive cell therapy (ACT) using cancer reactive T cells can produce positive clinical responses in cancer patients. Nevertheless, several obstacles to the successful use of ACT for the treatment of cancer and other diseases remain. For example, expansion of the numbers of T cells may produce T cells with a terminally differentiated phenotype that is associated with diminished antitumor activity and poor capacity for long-term persistence in vivo. Accordingly, there is a need for improved methods of obtaining an isolated population of T cells for ACT.

### BRIEF SUMMARY OF THE INVENTION

An embodiment of the invention provides a method of producing an isolated population of T cells for adoptive cell therapy, the method comprising culturing isolated T cells having antigenic specificity for a cancer antigen in vitro in the presence of a p38 mitogen activated protein kinase (p38 MAPK) inhibitor.

Further embodiments of the invention provide related isolated populations of T cells, pharmaceutical compositions, and methods of treating or preventing cancer in a mammal.

### BRIEF DESCRIPTION OF THE SEVERAL VIEWS OF THE DRAWING(S)

FIG. 1A is a schematic illustrating examples of favorable T cell phenotypes for ACT following T cell receptor (TCR) stimulation, expansion and differentiation of the T cells.

FIG. 1B is a schematic illustrating an experiment carried out to test the ability of MAPK14 gene expression disruption to produce the phenotype of FIG. 1A.

FIG. 2A is a graph showing the pleiotropy rank scores for first and second screens of T cells in which MAPK14 expression was disrupted using CRISPR-Cas9.

FIG. 2B is a graph showing the cell counts achieved following disruption of the MAPK14 gene using the CRISPR-Cas9 system and MAPK14 sgRNA2 or sgRNA3. T cells treated with buffer, Cas9 alone, or B2m sgRNA served as controls.

FIG. 2C is a graph showing the reactive oxygen species (ROS) measured in CD8+ T cells following disruption of the MAPK14 gene using the CRISPR-Cas9 system and MAPK14 sgRNA2 or sgRNA3. T cells treated with buffer, Cas9 alone, or B2m sgRNA served as controls.

FIG. 3 is a schematic illustrating p38 MAPK signaling in T cells.

FIGS. 4A and 4B are graphs showing the proportion of cells with low levels of gamma H2AX and high levels of gamma H2AX with phosphorylated p38 MAPK.

FIGS. 4C and 4D are graphs showing the percentage of cells with high levels of gamma H2AX (4C) and the cell count (4D) following treatment with p38 inhibitor Birb796.

FIG. 4E shows flow cytometry dot plots illustrating the expression of CD44 and CD62L by T cells following treatment with p38 inhibitor Birb796 or vehicle.

FIG. 4F is a graph showing the percentage of the cell population with a T_{EM} (unshaded portion of bars) or T_{CM} (shaded portion of bars) phenotype following treatment with p38i.

FIGS. 4G and 4H are graphs showing the percentage of CD8+CD62L+ cells measured in cells from healthy donors (4G) or melanoma patients (4H) following treatment with p38i or vehicle.

FIG. 5A is a graph showing the levels of cell proliferation following (i) stimulation with gp100 antigen and feeders or anti-CD3/CD28 stimulation and (ii) treatment with vehicle or p38i, as measured by dilution of CSFE proliferation dye.

FIG. 5B shows flow cytometry dot plots illustrating the numbers of levels of cells which underwent apoptosis following treatment with vehicle or p38i as measured by apoptotic cell marker annexin.

FIG. 5C is a graph showing the percentage of non-apoptotic, live cells measured in cells treated with vehicle (A) or p38i (B).

FIG. 5D are graphs showing the levels of gamma H2AX, fold-change following expansion, and the percentage of T_{CM} cells following restimulation and treatment with vehicle (A) or p38i (B).

FIG. 5E shows flow cytometry dot plots illustrating the numbers of levels of cells which underwent apoptosis following restimulation and treatment with vehicle or p38i as measured by apoptotic cell marker Annexin.

FIG. 5F is a graph showing the percentage of non-apoptotic, live cells measured in cells following restimulation and treatment with vehicle (A) or p38i (B).

FIGS. 6A-6E are graphs showing the phosphorylation of p38 (A), Akt (T308) (B), S6 (C), Akt (S473) (D), and Erk1/2 (E) following treatment with vehicle or p38i.

FIGS. 6F-6J are graphs showing the phosphorylation of p38 (F), Akt (T308) (G), S6 (H), Akt (S473) (I), and Erk1/2 (J) following treatment with vehicle or p38i.

FIG. 7A is a graph showing the fold change in metabolites measured following treatment with vehicle or p38i. p38i is shown in the upper right quadrant of the graph and the vehicle (veh) is shown in the upper left and lower right and left quadrants.

FIG. 7B is a schematic illustrating glycolysis and the citric acid cycle.

FIG. 7C shows graphs illustrating the fold change in production of glucose, lactate, succinate, and alpha-ketoglutarate following treatment with vehicle or p38i. In each graph, the cluster on the left represents the vehicle (veh) and the cluster on the right represents p38i.

FIGS. 8A-8C are graphs showing the fold change in production of linoleate (18:2n6) (A), 9-HODE/13-HODE (B), and oxidized GSSG (C) following treatment with vehicle or p38i. In each graph, the cluster on the left represents the vehicle (veh) and the cluster on the right represents p38i.

FIG. 9A shows graphs illustrating the mRNA expression of catalase, Slc2a1, Fasn, and Cpt1a following treatment with vehicle (A) or p38(i) (B).

FIGS. 9B and 9C are graphs showing the total ROS (9B) and mitochondrial ROS (9C) following treatment with vehicle (A) or p38i (B).

FIG. 9D is a graph illustrating the O₂ consumption (pMoles) measured following treatment with vehicle or p38i.

FIGS. 10A-10C are graphs illustrating the percentage of cytokine (IFNγ (A), IL-2 (B), and TNFα (C)) positive cells measured following treatment with vehicle (A) or p38i (B).

FIGS. 10D-10F are graphs illustrating the percentages of cells from healthy donor numbers 1 (D), 2 (E), and 3 (F) expressing IFNγ following treatment with vehicle or p38i.

FIGS. 10G and 10H are graphs illustrating the tumor area (mm²) and survival of tumor-bearing mice at various time points (days (d)) following transfer of T cells which had been treated with vehicle or p38i into the mice. Untreated tumor-bearing mice served as a control.

FIG. 11 is a graph showing the specific cytolysis achieved by T cells from two different donors transduced with an anti-CD19 CAR and treated with p38i or not treated with p38i. T cells which were not transduced with a CAR served as a control.

### DETAILED DESCRIPTION OF THE INVENTION

It has been discovered that T cells treated with a p38 MAPK inhibitor maintain their capacity to target cancer antigen-expressing cells and have transcriptional, metabolic, and functional properties that correlate with enhanced in vivo persistence after adoptive-transfer. P38 MAPK inhibitor-treated cells have features of long-lived memory T cells that are associated with prolonged antitumor immunity.

In this regard, an embodiment of the invention provides a method of producing an isolated population of T cells for adoptive cell therapy, the method comprising culturing isolated T cells having antigenic specificity for a cancer antigen in vitro in the presence of a p38 MAPK inhibitor.

The method may comprise isolating T cells from a mammal by any suitable method known in the art. For example, the T cells can be obtained from the mammal by a blood draw or a leukapheresis. In an embodiment of the invention, the method comprises isolating peripheral blood lymphocyte (PBL) or a peripheral blood mononuclear cell (PBMC) from a mammal. Alternatively or additionally, the T cells can be obtained from a tumor sample taken from the mammal. In this regard, the T cells may be tumor infiltrating lymphocytes (TIL).

The population of T cells may include any type of T cells. For example, the T cells may be a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, tumor, or other tissues or fluids. T cells can also be enriched for or purified. The T cell may be a human T cell. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating lymphocytes (TIL), memory T cells, naïve T cells, and the like. The T cell may be a CD8⁺ T cell or a CD4⁺ T cell.

Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). It is preferred that the mammals are non-human primates, e.g., of the order Primates, Ceboids, or Simoids (monkeys) or of the order Anthropoids (humans and apes). In some embodiments, the mammal may be a mammal of the order Rodentia, such as mice and hamsters. In other embodiments, the mammal is not a mouse. Preferably, the mammal is a non-human primate or a human. An especially preferred mammal is the human.

The method comprising culturing tumor fragments, isolated PBMC, or PBL (e.g., T cells) in vitro in the presence of one or more p38 MAPK inhibitors. The p38 MAPK inhibitor may be any suitable p38 MAPK inhibitor. The p38 MAPK inhibitor may be an allosteric inhibitor or a non-allosteric inhibitor of p38 MAPK. The p38 MAPK inhibitor may be p38 MAPK isoform-specific or non-p38 MAPK isoform specific. P38 MAPK (also referred to as mitogen-activated protein kinase 14 or MAPK14) has four isoforms: p38 MAPK-alpha (α), p38 MAPK-beta (β), p38 MAPK-gamma (γ), and p38 MAPK-delta (δ). The p38 MAPK inhibitor may inhibit any one or more of p38 MAPK-α, p38 MAPK-β, p38 MAPK-γ, and p38 MAPK-δ. The isoforms p38 MAPK-α and p38 MAPK-β are expressed by T-cells. In a preferred embodiment, the p38 MAPK inhibitor inhibits one or both of p38 MAPK-α and p38 MAPK-β.

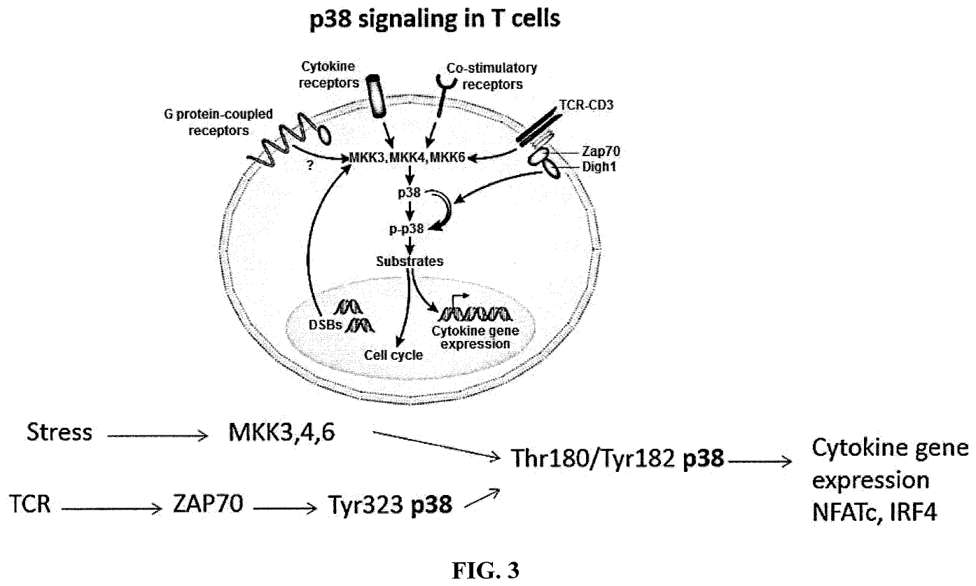

The p38 MAPK inhibitor can be any agent that inhibits the biological activity of p38 MAPK. P38 MAPK is responsive to stress stimuli such as, for example, cytokines, ultraviolet irradiation, heat shock, and osmotic shock. Without being bound to a particular theory or mechanism, it is believed that one or more of MKK3, MKK4, and MKK6 may activate p38 MAPK by phosphorylation at residues Thr180 and Tyr182 of p38 MAPK in response to a stress stimulus. Alternatively or additionally, it is believed that ZAP70 may activate p38 MAPK by phosphorylation at residue Tyr323 of p38 MAPK in response to the activation of a TCR. Activated p38 may activate the expression of one or more of cytokines, NFATc, and IRF4. A schematic illustrating p38 signaling in T cells is shown in FIG. 3.

The biological activity of p38 MAPK may be inhibited in any manner, e.g., by inhibiting the expression of one or both of p38 MAPK mRNA and p38 MAPK protein; by inhibiting the binding of p38 MAPK to its substrate; and/or by inhibiting p38 MAPK signaling, as compared to that which is observed in the absence of the p38 MAPK inhibitor. The biological activity may be inhibited to any degree that provides T cells with a beneficial therapeutic effect. For example, in some embodiments, the biological activity may be completely inhibited (i.e., prevented), while in other embodiments, the biological activity may be partially inhibited (i.e., reduced). As used herein, unless stated otherwise, the term “p38 MAPK” refers to p38 MAPK in any form (e.g., mRNA or protein) and from any species (e.g., human or mouse).

In an embodiment of the invention, the p38 MAPK inhibitor is an agent that inhibits p38 MAPK signaling. P38 MAPK signaling can be inhibited in any manner. For example, the p38 MAPK inhibitor may inhibit the activation or activity of any one or more of various downstream targets of p38 MAPK signaling (e.g., expression of cytokine genes, NFATc, IRF4). For example, the p38 MAPK inhibitor may be an agent that binds to the p38 MAPK protein, thereby reducing or preventing p38 MAPK signaling and inhibiting its function. By way of illustration, the agent that inhibits p38 MAPK signaling can be any of the agents which inhibit p38 MAPK expression, pharmacological inhibitors, or peptide (or polypeptide) inhibitors described herein.

In an embodiment, the p38 MAPK inhibitor is an agent that inhibits the binding of p38 MAPK to a p38 MAPK substrate. The substrates of p38 MAPK may include, for example, any one or more of the transcription regulator ATF2, MEF2C, and MAX, the cell cycle regulator CDC25B, and the tumor suppressor p53. In this regard, the p38 MAPK inhibitor may be an agent that binds to the p38 MAPK protein or the p38 MAPK substrate, thereby reducing or preventing the binding of the p38 MAPK protein to the p38 MAPK substrate and inhibiting its function, as well as agents that compete with the p38 MAPK protein for the native p38 MAPK binding site of the p38 MAPK substrate. By way of illustration, the agent that inhibits the binding of p38 MAPK to the p38 MAPK substrate can be any of the agents which inhibit p38 MPAK expression, pharmaceutical inhibitors, or peptide inhibitors described herein.

Inhibitors of p38 MAPK may include pharmacological inhibitors (e.g., small molecules) and peptides or polypeptides that inhibit p38 MAPK signaling, bind the p38 MAPK or p38 MAPK substrate protein or functional fragment thereof, compete with the p38 MAPK protein or functional fragment thereof for its native binding site of the p38 MAPK substrate, or complete with the p38 MAPK substrate for its native binding site of p38 MAPK. Suitable inhibitors can include, for example, chemical compounds or a non-active fragment or mutant of a p38 MAPK protein. In this regard, in an embodiment of the invention, the p38 MAPK inhibitor is a mutated p38 MAPK. The mutation may include any insertions, deletions, and/or substitutions of one or more amino acids in any position of the p38 MAPK protein that effectively inhibits p38 MAPK biological activity (e.g., p38 MAPK signaling and/or binding of p38 MAPK to a p38 MAPK substrate). For example, the p38 MAPK inhibitor can bind to the p38 MAPK substrate and/or inhibit p38 MAPK signaling.

In an embodiment of the invention, the p38 MAPK inhibitor is a pharmacological p38 MAPK inhibitor. Examples of pharmacological p38 MAPK inhibitors that may be useful in the inventive methods include, but are not limited to, BIRB 796 (doramapimod), SB203580, SB202190 (FHPI), SB 239063, and LY2228820. Preferably, the p38 MAPK inhibitor is BIRB 796 (doramapimod). BIRB 796 is a pan-p38 MAPK inhibitor with an IC₅₀ of about 38 nM, about 65 nM, about 200 nM, and about 520 nM for the α, β, γ, and δ isoforms of p38 MAPK, respectively, in cell-free assays.

Inhibitors of p38 MAPK can be identified using routine techniques. For example, inhibitors can be tested in binding assays to identify molecules and peptides (or polypeptides) that bind to p38 MAPK or a p38 MAPK substrate with sufficient affinity to inhibit p38 MAPK biological activity (e.g., binding of p38 MAPK to a p38 MAPK substrate, and/or p38 MAPK signaling). Also, competition assays can be performed to identify small molecules and peptides (or polypeptides) that inhibit the activation of downstream targets of p38 MAPK signaling or compete with p38 MAPK or functional fragment thereof for binding to its native binding site of a p38 MAPK substrate. Such techniques could be used in conjunction with mutagenesis of the p38 MAPK protein or functional fragment thereof itself, and/or with high-throughput screens of known pharmacological inhibitors.

The functional fragment of the p38 MAPK protein or p38 MAPK substrate protein can comprise any contiguous part of the p38 MAPK protein or p38 MAPK substrate protein that retains a relevant biological activity of the p38 MAPK or p38 MAPK substrate protein, e.g., binds to a p38 MAPK substrate or p38 MAPK and/or participates in p38 MAPK signaling. Any given fragment of a p38 MAPK or p38 MAPK substrate protein can be tested for such biological activity using methods known in the art. For example, the functional fragment can comprise, consist essentially of, or consist of the p38 MAPK substrate binding portion of the p38 MAPK protein or the p38 MAPK binding portion of the p38 MAPK substrate protein. In reference to the parent p38 MAPK or p38 MAPK substrate protein, the functional fragment preferably comprises, for instance, about 10% or more, about 25% or more, about 30% or more, about 50% or more, about 60% or more, about 80% or more, about 90% or more, or even about 95% or more of the parent p38 MAPK protein or p38 MAPK substrate protein, respectively.

In an embodiment of the invention, the p38 MAPK inhibitor is any suitable agent that inhibits the expression of one or both of p38 MAPK mRNA and p38 MAPK protein. The p38 MAPK inhibitor can be a nucleic acid at least about 10 nucleotides in length that specifically binds to and is complementary to a target nucleic acid encoding one or both of p38 MAPK mRNA and p38 MAPK protein, or a complement thereof. The p38 MAPK inhibitor may be introduced into a T cell, wherein the cell is capable of expressing one or both of p38 MAPK mRNA and p38 MAPK protein, in an effective amount for a time and under conditions sufficient to interfere with expression of one or both of p38 MAPK mRNA and p38 MAPK protein, respectively.

In an embodiment of the invention, the p38 MAPK inhibitor may be an artificially engineered nuclease that inhibits expression of p38 MAPK. For example, p38 MAPK expression may be inhibited in a T cell using a genome editing technique. Genome editing techniques can modify gene expression in a target cell by inserting, replacing, or removing DNA in the genome using an artificially engineered nuclease. Examples of such nucleases may include zinc finger nucleases (ZFNs) (Gommans et al., J Mol. Biol., 354(3): 507-519 (2005)), transcription activator-like effector nucleases (TALENs) (Zhang et al., Nature Biotechnol., 29: 149-153 (2011)), the CRISPR/Cas system (Cheng et al., Cell Res., 23: 1163-71 (2013)), and engineered meganucleases (Riviere et al., Gene Ther., 21(5): 529-32 (2014)). The nucleases create specific double-stranded breaks (DSBs) at targeted locations in the genome, and use endogenous mechanisms in the cell to repair the induced break by homologous recombination (HR) and nonhomologous end-joining (NHEJ). Such techniques may be used to achieve inhibition of p38 MAPK in T cells. Accordingly, in an embodiment of the invention, the p38 MAPK inhibitor is/are CRISPR-Cas agent(s), zinc finger agent(s), or TALEN agent(s). The TALEN agent(s) may comprise transcription activator-like effectors (TALES) which bind to the p38 MAPK gene and a TALEN. The zinc finger agent(s) may comprise zinc-finger nucleases which bind to the p38 MAPK gene.

In an embodiment of the invention, the methods employ the CRISPR/Cas system. Accordingly, the inventive method may comprise introducing a nucleic acid encoding a Cas endonuclease and a nucleic acid encoding a single guide RNA (sgRNA) molecule into a T cell, wherein the sgRNA hybridizes to the p38 MAPK gene in the T cell, and forming a complex between the sgRNA and Cas endonuclease so that the Cas endonuclease introduces a double strand break in the p38 MAPK gene. Non-limiting examples of Cas endonucleases include Casl B, Cas2, Cas3, Cas4, Cas5, Cas6, Cas7, Cas8, Cas9 (also known as Csnl and Csxl2), CaslO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, and Csxl7. Preferably, the Cas endonuclease is Cas9. Preferably, the sgRNA specifically hybridizes to the p38 MAPK gene such that the sgRNA hybridizes to the p38 MAPK gene and does not hybridize to any other gene that is not the p38 MAPK gene. Accordingly, in an embodiment of the invention, the p38 MAPK inhibitor may be CRISPR-Cas agent(s). The CRISPR-Cas agent(s) may comprise a nucleic acid encoding a Cas endonuclease and a nucleic acid encoding a single guide RNA (sgRNA) molecule into a T cell, wherein the sgRNA hybridizes to the p38 MAPK gene in the T cell.

The method may further comprise deleting all or a portion of the p38 MAPK gene to decrease expression of the p38 MAPK gene. The expression of the p38 MAPK gene may be decreased by any amount, for example, by about 10%, about 20%, about 30%, about 40%, about 50%, about 60%, about 70%, about 80%, or about 90%. Preferably, expression of the p38 MAPK gene is decreased so that there is no detectable expression of the p38 MAPK gene.

In some embodiments, RNA interference (RNAi) is employed. In this regard, the p38 MAPK inhibitor may comprise an RNAi agent. In an embodiment, the RNAi agent may comprise a small interfering RNA (siRNA), a short hairpin miRNA (shMIR), a microRNA (miRNA), or an antisense nucleic acid.

The sgRNA or RNAi agent, e.g., siRNA, shRNA, miRNA, and/or antisense nucleic acid can comprise overhangs. That is, not all nucleotides need bind to the target sequence. The sgRNA or RNAi nucleic acids employed can be at least about 19, at least about 40, at least about 60, at least about 80, at least about 100, at least about 120, at least about 140, at least about 160, at least about 180, at least about 200, at least about 220, at least about 240, from about 19 to about 250, from about 40 to about 240, from about 60 to about 220, from about 80 to about 200, from about 60 to about 180, from about 80 to about 160, and/or from about 100 to about 140 nucleotides in length.

The sgRNA or RNAi agent, e.g., siRNA or shRNA, can be encoded by a nucleotide sequence included in a cassette, e.g., a larger nucleic acid construct such as an appropriate vector. Examples of such vectors include lentiviral and adenoviral vectors, as well as other vectors described herein with respect to other aspects of the invention. An example of a suitable vector is described in Aagaard et al. Mol. Ther., 15(5): 938-45 (2007). When present as part of a larger nucleic acid construct, the resulting nucleic acid can be longer than the comprised sgRNA or RNAi nucleic acid, e.g., greater than about 70 nucleotides in length. In some embodiments, the RNAi agent employed cleaves the target mRNA. In other embodiments, the RNAi agent employed does not cleave the target mRNA.

Any type of suitable sgRNA, siRNA, miRNA, and/or antisense nucleic acid can be employed. In an embodiment, the antisense nucleic acid comprises a nucleotide sequence complementary to at least about 8, at least about 15, at least about 19, or from about 19 to about 22 nucleotides of a nucleic acid encoding one or both of p38 MAPK mRNA and p38 MAPK protein or a complement thereof. In an embodiment, the siRNA may comprise, e.g., trans-acting siRNAs (tasiRNAs) and/or repeat-associated siRNAs (rasiRNAs). In another embodiment, the miRNA may comprise, e.g., a short hairpin miRNA (shMIR).

In an embodiment of the invention, the p38 MAPK inhibitor may inhibit or downregulate to some degree the expression of the protein encoded by a p38 MAPK gene, e.g., at the DNA, RNA, or other level of regulation. In this regard, a T cell comprising a p38 MAPK inhibitor expresses none of one or both of p38 MAPK mRNA and p38 MAPK protein or lower levels of one or both of p38 MAPK mRNA and p38 MAPK protein as compared to a T cell that lacks a p38 MAPK inhibitor. In accordance with an embodiment of the invention, the p38 MAPK inhibitor can target a nucleotide sequence of a p38 MAPK gene or mRNA encoded by the same. Examples of human p38 MAPK sequences are set forth in Table 1. Other p38 MAPK sequences can be employed in accordance with the invention. Human and mouse antisense nucleic acids are commercially available (e.g., from OriGene Technologies, Inc., Rockville, Md. or Sigma-Aldrich, St. Louis, Mo.) and can be prepared using the nucleic acid sequences encoding the p38 MAPK proteins disclosed herein and routine techniques.

TABLE 1

| Genbank | |
|---|---|
| Accession No. | Name |
| NP_001306.1 | Human mitogen-activated protein kinase 14 |
| | isoform 1 (SEQ ID NO: 1) |
| Gene ID: 1432 | Human p38 MAPK (MAPK14) gene (SEQ ID NO: 2) |
| NM_001315.2 | Human mitogen-activated protein kinase 14 |
| | isoform 1 (SEQ ID NO: 3) |
| NM_139012.2 | Human mitogen-activated protein kinase 14 |
| | isoform 2 (SEQ ID NO: 4) |
| NP_620581.1 | Human mitogen-activated protein kinase 14 |
| | isoform 2 (SEQ ID NO: 5) |
| NM_139013.2 | Human mitogen-activated protein kinase 14 |
| | isoform 3 (SEQ ID NO: 6) |
| NP_620582.1 | Human mitogen-activated protein kinase 14 |
| | isoform 3 (SEQ ID NO: 7) |
| NM_139014.2 | Human mitogen-activated protein kinase 14 |
| | isoform 4 (SEQ ID NO: 8) |
| NP_620583.1 | Human mitogen-activated protein kinase 14 |
| | isoform 4 (SEQ ID NO: 9) |

In accordance with an embodiment of the invention, the p38 MAPK inhibitor can target a nucleotide sequence selected from the group consisting of the 5′ untranslated region (5′ UTR), the 3′ untranslated region (3′ UTR), and the coding sequence of p38 MAPK, complements thereof, and any combination thereof. Any suitable p38 MAPK target sequence can be employed. In an embodiment of the invention, the sequences of the p38 MAPK inhibitor can be designed against a human p38 MAPK with the sequence of SEQ ID NO: 2 but also recognize the sequence of NM_139012.2 (or vice-versa). In an embodiment of the invention, the sequences of the p38 MAPK inhibitor can be designed against any one of the five nucleotide sequences set forth in Table 1, but also recognize any one or more of the other four nucleotide sequences set forth in Table 1. The p38 MAPK inhibitors can be designed against any appropriate p38 MAPK DNA or mRNA sequence.

In still another embodiment of the invention, the p38 MAPK inhibitor is an agent(s) which epigenetically inhibits expression of p38 MAPK. Epigenetic modification of gene expression involves changes in gene expression that do not involve changes to the underlying DNA sequence. Epigenetic modification involves a change in phenotype without a change in genotype which, in turn, affects how cells read the gene.

The p38 MAPK inhibitor can be obtained by methods known in the art. For example, p38 MAPK inhibitors that are peptides or polypeptides can be obtained by de novo synthesis as described in references, such as Chan et al., Fmoc Solid Phase Peptide Synthesis, Oxford University Press, Oxford, United Kingdom, 2005. Also, p38 MAPK inhibitors can be recombinantly produced using standard recombinant methods. See, for instance, Green and Sambrook, Molecular Cloning: A Laboratory Manual, 4th ed., Cold Spring Harbor Press, Cold Spring Harbor, N.Y. 2012. Further, the p38 MAPK inhibitor can be isolated and/or purified from a natural source, e.g., a human. Methods of isolation and purification are well-known in the art. In this respect, the p38 MAPK inhibitors may be exogenous and can be synthetic, recombinant, or of natural origin.

The p38 MAPK inhibitors that are peptides or polypeptides can be glycosylated, amidated, carboxylated, phosphorylated, esterified, N-acylated, cyclized via, e.g., a disulfide bridge, or converted into an acid addition salt and/or optionally dimerized or polymerized, or conjugated. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Of course, the method of the invention can comprise administering two or more p38 MAPK inhibitors, any of which may be the same or different from one another. Furthermore, the p38 MAPK inhibitor can be provided as part of a larger polypeptide construct. For instance, the p38 MAPK inhibitor can be provided as a fusion protein comprising a p38 MAPK inhibitor along with other amino acid sequences or a nucleic acid encoding same. The p38 MAPK inhibitor also can be provided as part of a conjugate or nucleic acid encoding same.

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

Nucleic acids encoding the p38 MAPK inhibitor (and degenerate nucleic acid sequences encoding the same amino acid sequences), can be constructed based on chemical synthesis and/or enzymatic ligation reactions using procedures known in the art. See, for example, Green et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides).

The T cells may be cultured in the presence of the p38 MAPK inhibitor in any suitable manner. In an embodiment of the invention, the T cells are cultured in the presence of a cytokine such as, for example, interleukin-2 (IL-2), interleukin-7 (IL-7), interleukin-15 (IL-15), interleukin-12 (IL-12) or a combination of two or more of the foregoing.

In an embodiment of the invention, the method further comprises introducing a nucleic acid encoding an exogenous T cell receptor (TCR) into the T cells in the presence of a p38 MAPK inhibitor and under conditions to express the TCR by the T cells. By “exogenous” is meant that the TCR is not native to (naturally-occurring on) the T cell. The exogenous TCR may be a recombinant TCR. A recombinant TCR is a TCR which has been generated through recombinant expression of one or more exogenous TCR α-, β-, γ-, and/or δ-chain encoding genes. A recombinant TCR can comprise polypeptide chains derived entirely from a single mammalian species, or the recombinant TCR can be a chimeric or hybrid TCR comprised of amino acid sequences derived from TCRs from two different mammalian species. For example, the antigen-specific TCR can comprise a variable region derived from a murine TCR, and a constant region of a human TCR such that the TCR is “humanized.” Any exogenous TCR having antigenic specificity for a cancer antigen may be useful in the inventive methods. The TCR generally comprises two polypeptides (i.e., polypeptide chains), such as an α-chain of a TCR, a β-chain of a TCR, a γ-chain of a TCR, a δ-chain of a TCR, or a combination thereof. Such polypeptide chains of TCRs are known in the art. The cancer antigen-specific TCR can comprise any amino acid sequence, provided that the TCR can specifically bind to and immunologically recognize a cancer antigen or epitope thereof. Examples of exogenous TCRs that may be useful in the inventive methods include, but are not limited to, those disclosed in, for example, U.S. Pat. Nos. 7,820,174; 7,915,036; 8,088,379; 8,216,565; 8,431,690; 8,613,932; 8,785,601; 9,128,080; 9,345,748; 9,487,573 and U.S. Patent Application Publication Nos. 2013/0116167; 2014/0378389; and 2015/0246959, each of which is incorporated herein by reference.

In an embodiment of the invention, the method further comprises introducing a nucleic acid encoding a chimeric antigen receptor (CAR) into the T cells in the presence of a p38 MAPK inhibitor and under conditions to express the CAR by the T cells. Typically, a CAR comprises the antigen binding domain of an antibody, e.g., a single-chain variable fragment (scFv), fused to the transmembrane and intracellular domains of a TCR. Thus, the antigenic specificity of a TCR of the invention can be encoded by a scFv which specifically binds to the cancer antigen, or an epitope thereof. Any CAR having antigenic specificity for a cancer antigen may be useful in the inventive methods. Examples of CARs that may be useful in the inventive methods include, but are not limited to, those disclosed in, for example, U.S. Pat. Nos. 8,465,743; 9,266,960; 9,765,342; 9,359,447 and U.S. Patent Application Publication Nos. 2014/0274909 and 2017/0107286, each of which is incorporated herein by reference.

A T cell comprising an endogenous cancer antigen-specific TCR can also be transformed, e.g., transduced or transfected, with one or more nucleic acids encoding an exogenous (e.g., recombinant) TCR or other recombinant receptor. Such exogenous receptors, e.g., TCRs, can confer specificity for additional antigens to the transformed T cell beyond the antigens for which the endogenous TCR is naturally specific. This can, but need not, result in the production of T cell having dual antigen specificities.

In a preferred embodiment, the exogenous TCR or CAR has antigenic specificity for a cancer antigen. The phrases “antigen-specific” and “antigenic specificity,” as used herein, mean that the TCR or CAR can specifically bind to and immunologically recognize an antigen, or an epitope thereof, such that binding of the TCR or CAR to antigen, or the epitope thereof, elicits an immune response.

In an embodiment of the invention, a nucleic acid encoding the exogenous TCR or CAR is introduced into any suitable recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector. Examples of recombinant expression vectors that may be useful in the inventive methods include, but are not limited to, plasmids, viral vectors (retroviral vectors, gamma-retroviral vectors, or lentiviral vectors), and transposons. The vector may then, in turn, be introduced into the isolated population of T cells by any suitable technique such as, e.g., gene editing, transfection, transformation, or transduction as described, for example, Green and Sambrook, Molecular Cloning: A Laboratory Manual (4^{th} Ed.), Cold Spring Harbor Laboratory Press (2012). Many transfection techniques are known in the art and include, for example, calcium phosphate DNA co-precipitation; DEAE-dextran; electroporation; cationic liposome-mediated transfection; tungsten particle-facilitated microparticle bombardment; and strontium phosphate DNA co-precipitation. Phage or viral vectors can be introduced into host cells, after growth of infectious particles in suitable packaging cells, many of which are commercially available. Preferably, the vector is introduced into the isolated population of T cells in the presence of a p38 MAPK inhibitor.

The isolated population of T cells, into which a vector encoding the exogenous TCR or CAR has been introduced, can be cultured ex vivo under conditions to express the exogenous TCR or CAR, and then directly transferred into a mammal (preferably a human) affected by cancer. Such a cell transfer method is referred to in the art as “adoptive cell transfer” or “adoptive cell therapy” (ACT). Preferably, the T cells are cultured under conditions to express the exogenous TCR or CAR and in the presence of a p38 MAPK inhibitor.

The p38 MAPK inhibitor-treated population of T cells administered to the mammal can be allogeneic or autologous to the mammal. In “autologous” administration methods, cells are removed from a mammal, stored (and optionally modified), and returned back to the same mammal. In “allogeneic” administration methods, a mammal receives cells from a genetically similar, but not identical, donor. Preferably, the cells are autologous to the mammal. Autologous cells may, advantageously, reduce or avoid the undesirable immune response that may target an allogeneic cell such as, for example, graft-versus-host disease.

While the T cells may be cultured in the presence of a p38 MAPK inhibitor intermittently in vitro, in a preferred embodiment of the invention, the T cells are cultured in the presence of the p38 MAPK inhibitor for the entire duration of in vitro culture, including during expansion of the numbers of cells and during introduction of a nucleic acid encoding a CAR or an exogenous TCR into the cells.

The T cells may have antigenic specificity for a cancer antigen. The term “cancer antigen,” as used herein, refers to any molecule (e.g., protein, polypeptide, peptide, lipid, carbohydrate, etc.) solely or predominantly expressed or over-expressed by a tumor cell or cancer cell, such that the antigen is associated with the tumor or cancer. The cancer antigen can additionally be expressed by normal, non-tumor, or non-cancerous cells. However, in such cases, the expression of the cancer antigen by normal, non-tumor, or non-cancerous cells is not as robust as the expression by tumor or cancer cells. In this regard, the tumor or cancer cells can over-express the antigen or express the antigen at a significantly higher level, as compared to the expression of the antigen by normal, non-tumor, or non-cancerous cells. Also, the cancer antigen can additionally be expressed by cells of a different state of development or maturation. For instance, the cancer antigen can be additionally expressed by cells of the embryonic or fetal stage, which cells are not normally found in an adult host. Alternatively, the cancer antigen can be additionally expressed by stem cells or precursor cells, which cells are not normally found in an adult host. Examples of cancer antigens include, but are not limited to, mesothelin, CD19, CD22, CD30, CD70, CD276 (B7H3), gp100, MART-1, Epidermal Growth Factor Receptor Variant III (EGFRVIII), Vascular Endothelial Growth Factor Receptor 2 (VEGFR-2), TRP-1, TRP-2, tyrosinase, human papillomavirus (HPV) 16 E6, HPV 16 E7, NY-BR-1, NY-ESO-1 (also known as CAG-3), SSX-2, SSX-3, SSX-4, SSX-5, SSX-9, SSX-10, MAGE-A1, MAGE-A2, BRCA, MAGE-A3, MAGE-A4, MAGE-A5, MAGE-A6, MAGE-A7, MAGE-A8, MAGE-A9, MAGE-A10, MAGE-A11, MAGE-A12, HER-2, etc. In an embodiment of the invention, the cancer antigen may be a mutated antigen that is expressed or overexpressed by tumor or cancer cells and which is not expressed by normal, non-tumor, or non-cancerous cells. Examples of such cancer antigens may include, but are not limited to, mutated KRAS and mutated p53. T cells having antigenic specificity for a cancer antigen may, advantageously, reduce or avoid cross-reactivity with normal tissues such as, for example, that which may occur using T cells having antigenic specificity for minor histocompatability antigens.

The cancer antigen can be an antigen expressed by any cell of any cancer or tumor, including the cancers and tumors described herein. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, the cancer antigen may be expressed by both breast and prostate cancer cells and not expressed at all by normal, non-tumor, or non-cancer cells.

In an embodiment of the invention, the method comprises expanding the number of T cells in the presence of one or more non-specific T cell stimuli, one or more cytokines, and the p38 MAPK inhibitor. Examples of non-specific T cell stimuli include, but are not limited to, one or more of irradiated allogeneic feeder cells, irradiated autologous feeder cells, anti-CD3 antibodies, anti-4-1BB antibodies, and anti-CD28 antibodies. In preferred embodiment, the non-specific T cell stimulus may be anti-CD3 antibodies and anti-CD28 antibodies conjugated to beads. Any one or more cytokines may be used in the inventive methods. Exemplary cytokines that may be useful for expanding the numbers of cells include interleukin (IL)-2, IL-7, IL-21, and IL-15. The p38 MAPK inhibitor for expanding the numbers of cells may be as described herein with respect to other aspects of the invention.

Expansion of the numbers of T cells can be accomplished by any of a number of methods as are known in the art as described in, for example, U.S. Pat. Nos. 8,034,334; 8,383,099; and U.S. Patent Application Publication No. 2012/0244133. In an embodiment of the invention, the numbers of T cells are expanded by physically contacting the T cells with one or more non-specific T cell stimuli and one or more cytokines in the presence of the p38 MAPK inhibitor. For example, expansion of the numbers of T cells may be carried out by culturing the T cells with OKT3 antibody, IL-2, and feeder PBMC (e.g., irradiated allogeneic PBMC) in the presence of the p38 MAPK inhibitor. In an embodiment of the invention, expanding the number of T cells in the presence of the p38 MAPK inhibitor comprises culturing the cells for at least about 14 days in the presence of the p38 MAPK inhibitor.

The invention further provides an isolated or purified population of T cells produced by any of the inventive methods. The p38 MAPK inhibitor-treated populations of T cells produced by the inventive methods may provide many advantages. For example, the p38 MAPK inhibitor-treated populations of T cells produced by the inventive methods may be less differentiated as compared to control T cells, wherein the control T cells are identical to the T cells that were cultured in the presence of a p38 MAPK inhibitor except that the control T cells were not cultured in the presence of a p38 MAPK inhibitor. The less differentiated populations of p38 MAPK inhibitor-treated T cells produced according to the inventive methods may, advantageously, demonstrate any one or more of greater persistence, proliferation, trafficking to tumor site(s), and antitumor activity upon in vivo transfer as compared to control T cells. For example, expansion of the numbers of T cells in the presence of a p38 MAPK inhibitor in accordance with the inventive methods may reduce or avoid the production of T cells with a terminally differentiated phenotype that is associated with diminished antitumor activity and poor capacity for long-term persistence in vivo.

In an embodiment of the invention, the p38 MAPK inhibitor-treated population of T cells produced by the inventive methods has an increased proportion of cells with a naïve T cell (T_{N}), T memory stem cell (T_{SCM}), or central memory T cell (T_{CM}) phenotype as compared to control T cells. The control T cells are as described herein with respect to other aspects of the invention. Alternatively or additionally, the p38 MAPK inhibitor-treated population of T cells has a decreased proportion of cells with an effector memory T cell (T_{EM}) phenotype as compared to the control T cells. For example, CCR7 and CD62L are expressed by T_{N}, T_{SCM}, and T_{CM} cells, but are not expressed by T_{EM} cells. The transcription factors LEFT, FOXP1, and KLF7 are expressed by T_{N}, T_{SCM}, and T_{CM} cells, but are not expressed by T_{EM} cells. CD45RO and KLRG1 are not expressed by T_{N} or T_{SCM} cells, but are expressed by T_{EM} cells. Gattinoni et al., Nat. Rev. Cancer, 12: 671-84 (2012). In an embodiment of the invention, the p38 MAPK inhibitor-treated isolated or purified T cell of the invention may be any one or more of CD62L⁺, KLRG1⁻, LEF1⁺, FOXP1⁺, and KLF7⁺, CCR7⁺, CD57⁺, and CD45RO⁻. The T cells may be CD62L⁺. Alternatively or additionally, the T cells may be CD8⁺. In an especially preferred embodiment, the p38 MAPK inhibitor-treated population of T cells is both CD62L⁺ and CD8⁺. Alternatively or additionally, T_{N}, T_{SCM}, and T_{CM} cells may be characterized by longer telomeres as compared to those of T_{EM} cells.

In an embodiment of the invention, the p38 MAPK inhibitor-treated population of T cells produced according to the inventive methods has an increased expression of genes associated with a T_{N}, T_{SCM}, or T_{CM} phenotype as compared to the control T cells. For example, the p38 MAPK inhibitor-treated population of T cells may have an increased expression of any one or more of catalase, Slc2a1, Il7r, Sell, CD28, CD27, Foxo1, Klf2, Tcf7, and Lef1 mRNA or protein as compared to control T cells, wherein the control T cells are as described herein with respect to other aspects of the invention. For example, the p38 MAPK inhibitor-treated populations of T cells produced according to the inventive methods may have a higher expression of CD27 and/or CD28 as compared to control T cells. Without being bound to a particular theory, it is believed that CD27 and CD28 are associated with proliferation, in vivo persistence, and a less differentiated state of T cells (the increased differentiation of T cells is believed to negatively affect the capacity of T cells to function in vivo). T cells expressing higher levels of CD27 are believed to have better antitumor activity than CD27-low cells.

In an embodiment of the invention, the p38 MAPK inhibitor-treated population of T cells produced according to the inventive methods has a decreased expression of genes associated with a T_{EM} phenotype. For example, the p38 MAPK inhibitor-treated population of T cells may have a decreased expression of any one or more of Eomes, Prfl, GzmB, and Klrg1 mRNA or protein as compared to control T cells, wherein the control T cells are as described herein with respect to other aspects of the invention. In an embodiment of the invention, expansion of the numbers of T cells in the presence of the p38 MAPK inhibitor, followed by restimulation of the T cells in the absence of the p38 MAPK inhibitor, results in higher Ifng production and enhanced anti-tumor activity.

Without being bound to a particular theory or mechanism, it is believed that (i) inhibition of glycolysis during ex vivo expansion of CD8⁺ T cells drives formation of immunological memory and enhances anti-tumor function and (ii) that inhibiting p38 MAPK may alter glycolytic activity of T cells and promote metabolic features associated with improved anti-tumor immunity.

The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70% or about 80%, about 90% or can be about 100%.

The p38 MAPK inhibitor-treated population of T cells produced by any of the inventive methods can be a heterogeneous population comprising the T cells produced by any of the inventive methods described herein, in addition to at least one other cell, e.g., a cell other than a T cell, e.g., a B cell, a macrophage, a neutrophil, an erythrocyte, a hepatocyte, an endothelial cell, an epithelial cell, a muscle cell, a brain cell, etc. Alternatively, the p38 MAPK inhibitor-treated population of cells can be a substantially homogeneous population, in which the population comprises mainly T cells produced by any of the inventive methods described herein. The p38 MAPK inhibitor-treated population also can be a clonal population of cells, in which all cells of the population are clones of a single T cell. In one embodiment of the invention, the population of cells is a clonal population comprising T cells comprising a recombinant expression vector encoding the exogenous TCR or CAR as described herein.

The inventive isolated or purified p38 MAPK inhibitor-treated population of T cells produced according to any of the inventive methods described herein may be included in a composition, such as a pharmaceutical composition. In this regard, the invention provides a pharmaceutical composition comprising the p38 MAPK inhibitor-treated isolated or purified population of T cells described herein and a pharmaceutically acceptable carrier.

Preferably, the carrier is a pharmaceutically acceptable carrier. With respect to pharmaceutical compositions, the carrier can be any of those conventionally used for the administration of cells. Such pharmaceutically acceptable carriers are well-known to those skilled in the art and are readily available to the public. It is preferred that the pharmaceutically acceptable carrier be one which has no detrimental side effects or toxicity under the conditions of use.

The choice of carrier may be determined in part by the particular method used to administer the p38 MAPK inhibitor-treated population of T cells. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the p38 MAPK inhibitor-treated population of T cells, and in certain instances, a particular route can provide a more immediate and more effective response than another route.

Preferably, the p38 MAPK inhibitor-treated population of T cells are administered by injection, e.g., intravenously. A suitable pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

For purposes of the invention, the dose, e.g., number of p38 MAPK inhibitor-treated T cells administered should be sufficient to effect, e.g., a therapeutic or prophylactic response, in the mammal over a reasonable time frame. For example, the number of p38 MAPK inhibitor-treated T cells administered should be sufficient to bind to a cancer antigen or treat or prevent cancer in a period of from about 2 hours or longer, e.g., 12 to 24 or more hours, from the time of administration. In certain embodiments, the time period could be even longer. The number of p38 MAPK inhibitor-treated T cells administered will be determined by, e.g., the efficacy of the particular population of T cells to be administered and the condition of the mammal (e.g., human), as well as the body weight of the mammal (e.g., human) to be treated.

Many assays for determining an administered number of p38 MAPK inhibitor-treated T cells are known in the art. For purposes of the invention, an assay, which comprises comparing the extent to which target cells are lysed or one or more cytokines such as, e.g., IFN-γ and IL-2 is secreted upon administration of a given number of such T cells to a mammal among a set of mammals of which is each given a different number of the T cells, could be used to determine a starting number to be administered to a mammal. The extent to which target cells are lysed or cytokines such as, e.g., IFN-γ and IL-2 are secreted upon administration of a certain number can be assayed by methods known in the art. Secretion of cytokines such as, e.g., IL-2, may also provide an indication of the quality (e.g., phenotype and/or effectiveness) of a T cell preparation.

The number of p38 MAPK inhibitor-treated T cells administered also will be determined by the existence, nature and extent of any adverse side effects that might accompany the administration of a particular population of T cells. Typically, the attending physician will decide the number of T cells with which to treat each individual patient, taking into consideration a variety of factors, such as age, body weight, general health, diet, sex, route of administration, and the severity of the condition being treated. By way of example and not intending to limit the invention, the number of p38 MAPK inhibitor-treated T cells to be administered can be about 10×10⁶ to about 10×10¹¹ cells per infusion, about 10×10⁹ cells to about 10×10¹¹ cells per infusion, or 10×10⁷ to about 10×10⁹ cells per infusion.

It is contemplated that the p38 MAPK inhibitor-treated populations of T cells produced according to the inventive methods can be used in methods of treating or preventing cancer in a mammal. In this regard, the invention provides a method of treating or preventing cancer in a mammal, comprising administering to the mammal any of the pharmaceutical compositions or populations of T cells described herein in an amount effective to treat or prevent cancer in the mammal.

One or more additional therapeutic agents can be coadministered to the mammal. By “coadministering” is meant administering one or more additional therapeutic agents and the p38 MAPK inhibitor-treated isolated population of T cells sufficiently close in time such that the p38 MAPK inhibitor-treated isolated population of T cells can enhance the effect of one or more additional therapeutic agents, or vice versa. In this regard, the p38 MAPK inhibitor-treated isolated population of T cells can be administered first and the one or more additional therapeutic agents can be administered second, or vice versa. Alternatively, the p38 MAPK inhibitor-treated isolated population of T cells and the one or more additional therapeutic agents can be administered simultaneously. Additional therapeutic agents that may enhance the function of the p38 MAPK inhibitor-treated isolated population of T cells may include, for example, one or more cytokines or one or more antibodies (e.g., antibodies that inhibit PD-1 function). An exemplary therapeutic agent that can be co-administered with the p38 MAPK inhibitor-treated isolated population of T cells is IL-2. Without being bound to a particular theory or mechanism, it is believed that IL-2 may enhance the therapeutic effect of the p38 MAPK inhibitor-treated isolated population of T cells.

An embodiment of the invention further comprises lymphodepleting the mammal prior to administering the p38 MAPK inhibitor-treated isolated population of T cells. Examples of lymphodepletion include, but may not be limited to, nonmyeloablative lymphodepleting chemotherapy, myeloablative lymphodepleting chemotherapy, total body irradiation, etc.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset or recurrence of the disease, or a symptom or condition thereof.

For purposes of the inventive methods, wherein populations of T cells are administered, the T cells can be cells that are allogeneic or autologous to the mammal. Preferably, the cells are autologous to the mammal.

With respect to the inventive methods, the cancer can be any cancer, including any of leukemia (e.g., B cell leukemia), sarcomas (e.g., synovial sarcoma, osteogenic sarcoma, leiomyosarcoma uteri, and alveolar rhabdomyosarcoma), lymphomas (e.g., Hodgkin lymphoma and non-Hodgkin lymphoma), hepatocellular carcinoma, glioma, head-neck cancer, acute lymphocytic cancer, acute myeloid leukemia, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer (e.g., colon carcinoma), esophageal cancer, cervical cancer, gastrointestinal carcinoid tumor, hypopharynx cancer, larynx cancer, liver cancer, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, rectal cancer, renal cancer, small intestine cancer, soft tissue cancer, stomach cancer, testicular cancer, thyroid cancer, ureter cancer, and urinary bladder cancer.

Another embodiment of the invention provides the T cell population isolated according to any of the inventive methods described herein for use in the treatment or prevention of cancer in a mammal.

The following examples further illustrate the invention but, of course, should not be construed as in any way limiting its scope.

### Example 1

This example demonstrates that knockout of p38 MAPK gene expression using CRISPR-Cas9 produces T cells with a favorable phenotype for ACT.

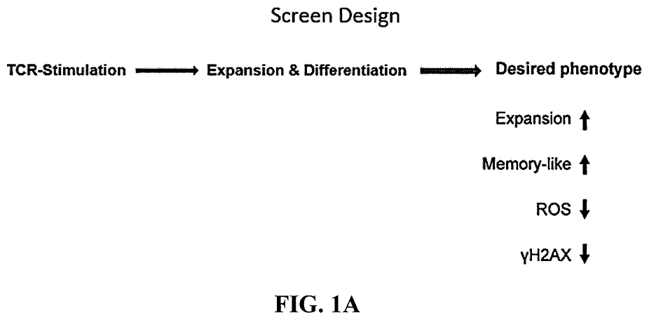

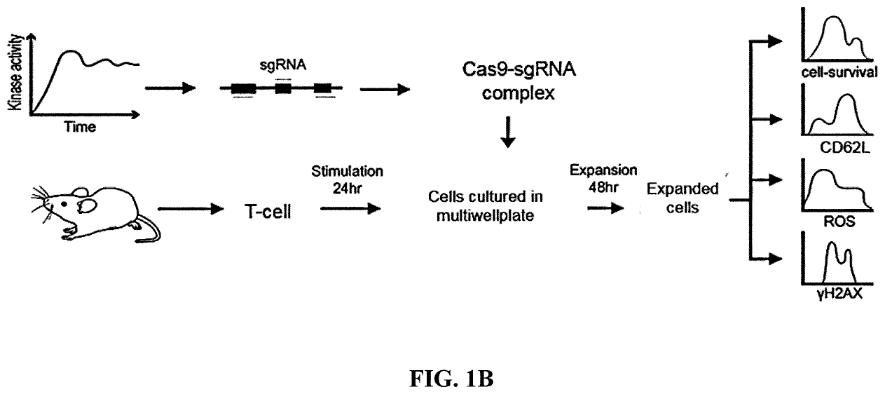

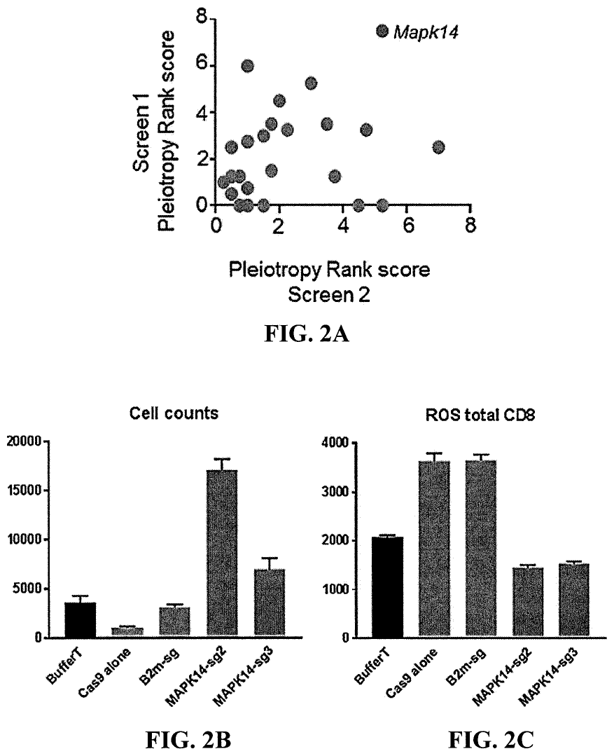

The MAPK14 gene encodes p38 MAPK. Expression of the MAPK14 gene was disrupted using the CRISPR-Cas9 system with MAPK14 sgRNA2 or sgRNA3 (FIGS. 1A and 1B). T cells treated with buffer, Cas9 alone, or B2m sgRNA served as controls. Pleiotropy rank score, cell counts, and the reactive oxygen species (ROS) of total CD8 T cells was measured. The results are shown in FIGS. 2A-2C. As shown in FIGS. 2A-2C, knockout of p38 MAPK gene expression using CRISPR-Cas9 produced T cells with a favorable phenotype for ACT.

### Example 2

This example demonstrates that inhibition of p38 MAPK augments genomic fitness, memory-like properties and expansion of the numbers of T cells.

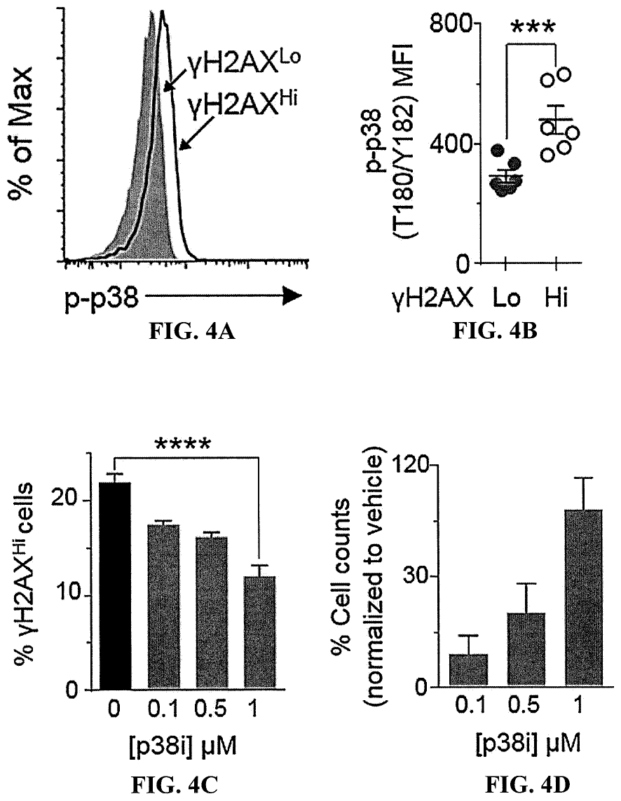

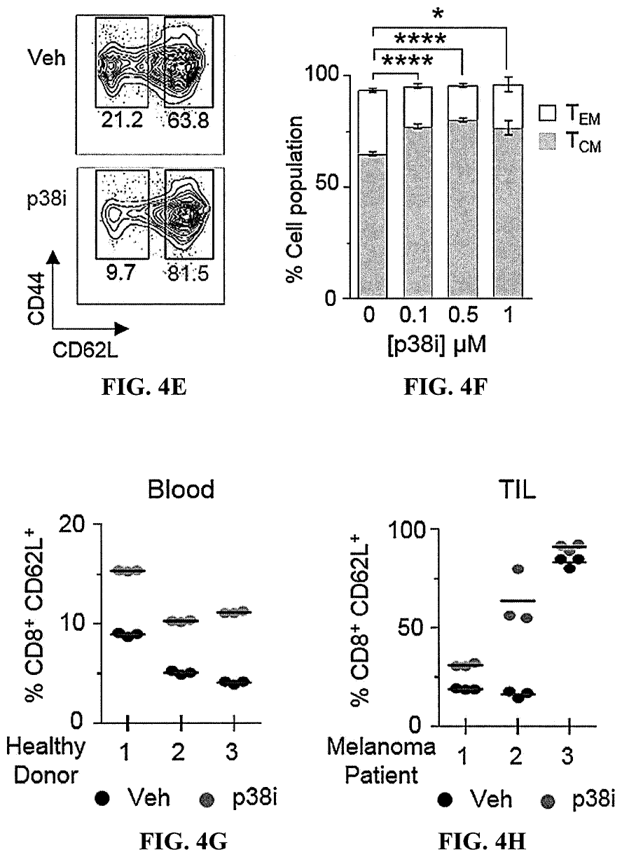

Mouse T cells were stimulated and the numbers of cells were expanded for 5 days in vitro. The level of activated phosphorylated p38 MAPK in cells with high or low levels of gamma H2AX (an indicator of DNA damage) in the T cells was measured. The results are shown in FIGS. 4A-4B. As shown in FIGS. 4A-4B, the subpopulation of T cells with high gamma H2AX contains higher amount of active phosphorylated p38.

Mouse T cells were stimulated and the numbers of cells were expanded for 5 days in vitro in the presence of the p38 inhibitor Birb796. The percentages of cells with high levels of gamma H2AX, the percentages of cell counts, and the expression of CD44 and CD62L were measured. The results are shown in FIGS. 4C-4F. As shown in FIGS. 4C-4F, with increases in the dosage of p38 inhibitor (p38i), the DNA damage in cells was reduced and the cell counts and percentage of CD62L+ cells were increased.

The numbers of human healthy donor derived T cells and melanoma patient-derived T cells were expanded for 20-25 days in presence of 0.5 uM p38i. The percentage of CD8+CD62L+ cells was measured. The results are shown in FIGS. 4G-4H. As shown in FIGS. 4G and 4H, the percentage of CD8+CD62L+ cells increased following treatment with p38i in both healthy donors and melanoma patients.

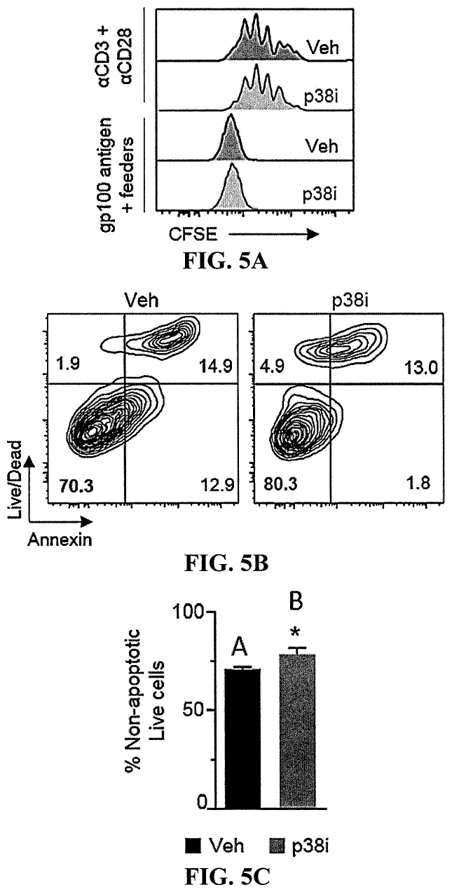

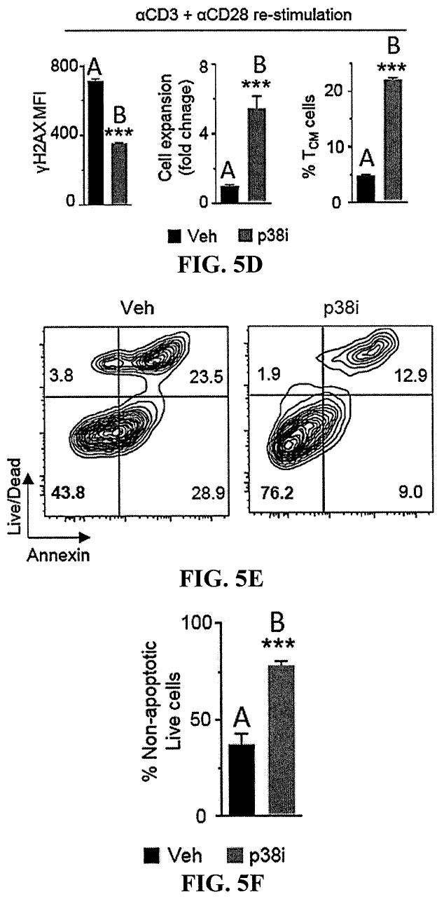

Mouse T cells were stimulated using either hgp100 peptide or anti-CD3/CD28 antibodies, and the numbers of cells were expanded for 4 days in vitro in the presence of 0.5 μM p38 inhibitor. Cell proliferation was measured. The results are shown in FIG. 5A. As shown in FIG. 5A, p38 inhibition did not affect cell proliferation as measured by dilution of CSFE proliferation dye.

Mouse T cells were stimulated and the numbers of cells were expanded for 4 days in vitro in the presence of 0.5 μM p38 inhibitor. The levels of cells which underwent apoptosis were measured. The results are shown in FIGS. 5B and 5C. As shown in FIGS. 5B and 5C, p38 inhibition reduced cell death during expansion as measured by apoptotic cell marker Annexin.

Mouse T cells were stimulated and the numbers of cells were expanded for 10 days in vitro in the presence of 0.5 μM p38 inhibitor. On day 5, the cells were re-stimulated. The levels of gamma H2AX, fold-change following expansion, the percentage of T_{CM} cells, and the proportion of apoptotic cells and non-apoptotic cells were measured. The results are shown in FIGS. 5D-5F. As shown in FIGS. 5D-5F, all desired phenotypes were enhanced upon TCR re-stimulation in T cells.

### Example 3

This example demonstrates that inhibition of p38 MAPK preserves memory-like characteristics of T cells independent of the Akt pathway.

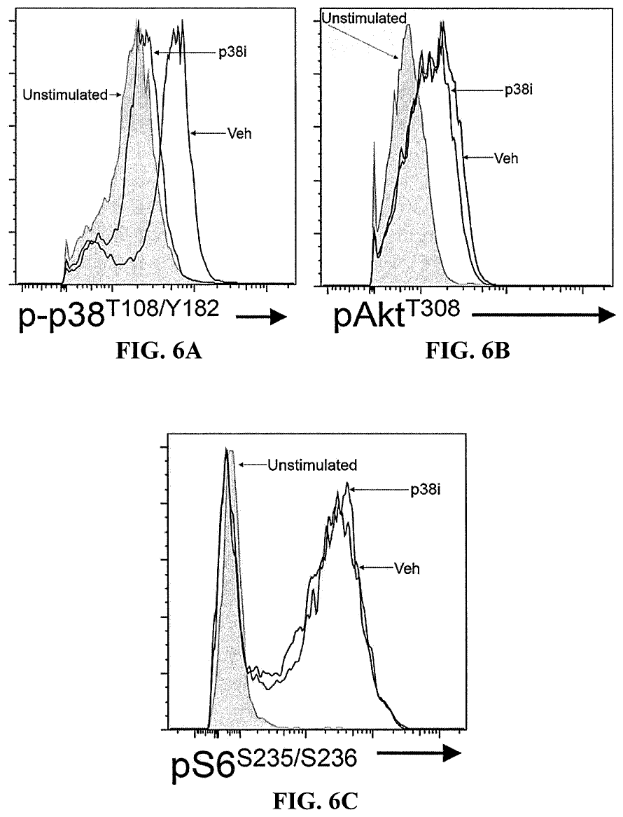

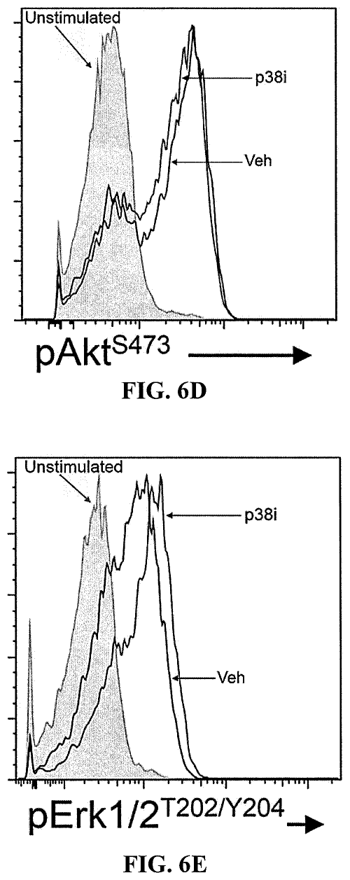

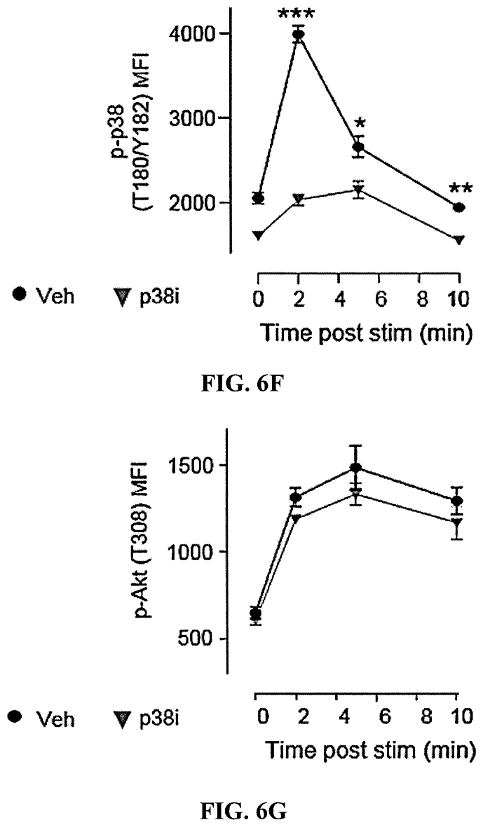

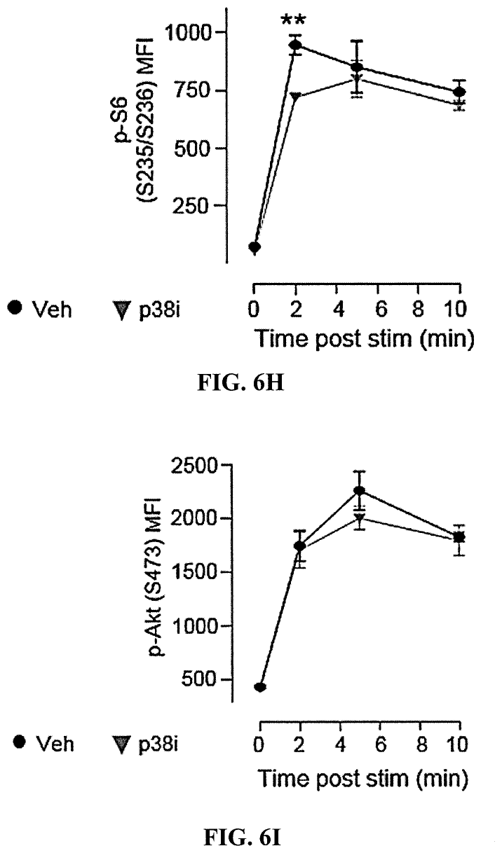

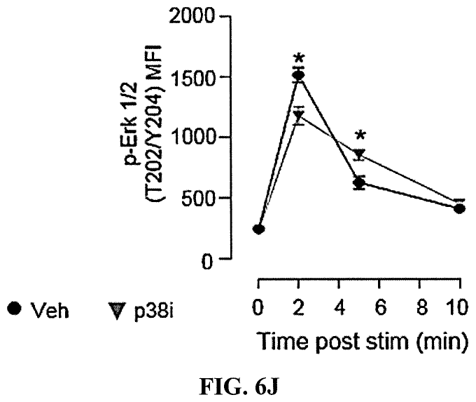

Mouse T cells were stimulated and the numbers of cells were expanded for 5 days in vitro in presence of 0.5 μM p38 inhibitor. Intracellular staining and FACS analysis was performed to measure phosphorylation of Akt and its substrates in T cells. The results are shown in FIGS. 6A-6J. As shown in FIGS. 6A-6J, inhibition of p38 MAPK preserved memory-like characteristics of T cells independent of the Akt pathway.

### Example 4

This example demonstrates that inhibition of p38 reduces genomic stress via unique cell-intrinsic metabolic alteration.

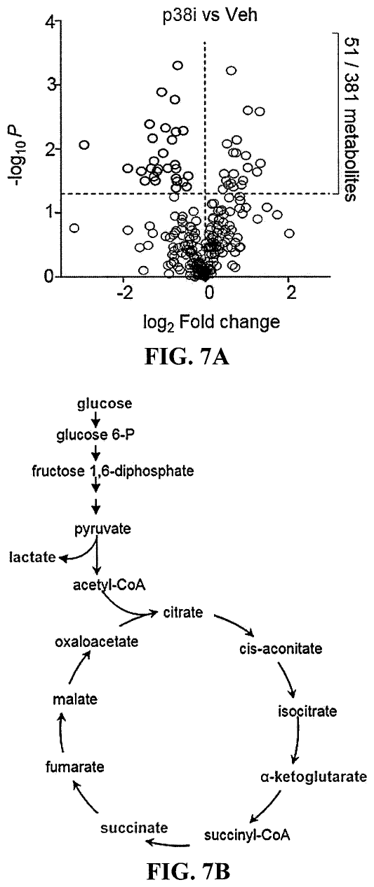

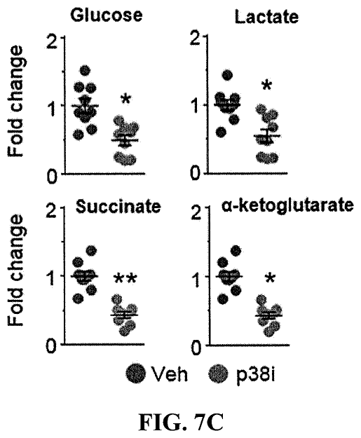

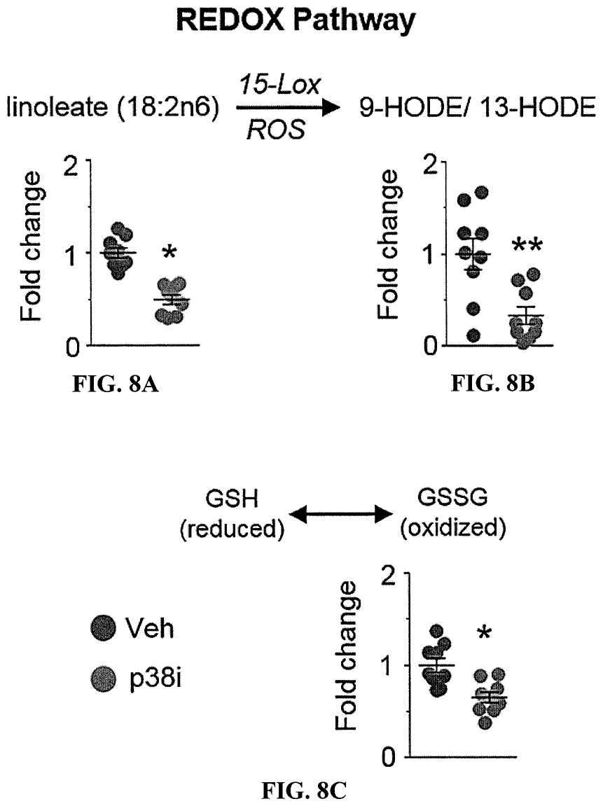

Mouse T cells were stimulated and the numbers of cells were expanded for 5 days in vitro in the presence of 0.5 μM p38 inhibitor. Cells were subjected to metabolome analysis to determine the levels of different metabolites to evaluate whether p38i alters metabolic pathways. The results are shown in FIGS. 7A and 7C and 8A-8C. FIG. 7B is a schematic illustrating glycolysis and the citric acid cycle. As shown in FIG. 7C, treatment with p38i reduced glucose, lactate, succinate, and alpha-ketoglutarate production. As shown in FIGS. 8A-8C, treatment with p38i reduced the production of linoleate (18:2n6), 9-HODE/13-HODE, and oxidized GSSG.

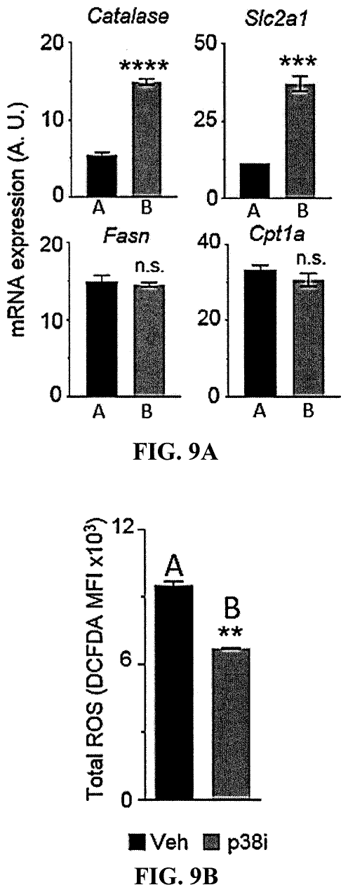

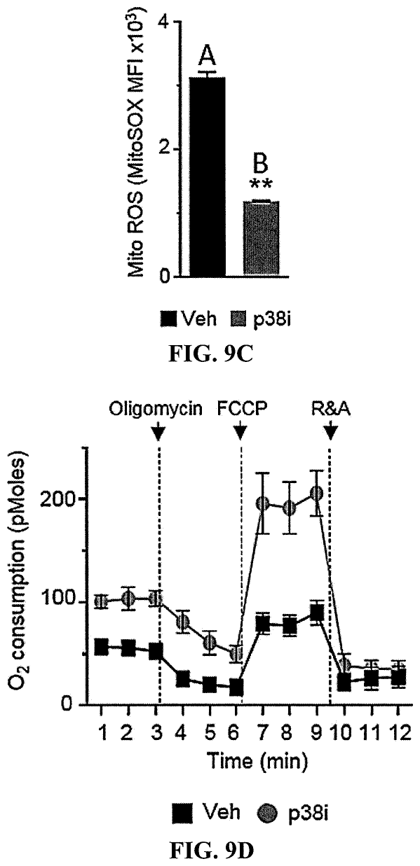

Mouse T cells were stimulated and expanded for 5 days in vitro in the presence of 0.5 μM p38 inhibitor. RNA extraction and qPCR analysis were performed to determine differences in gene expression. The results are shown in FIG. 9A. As shown in FIG. 9A, treatment with p38i reduced expression of catalase and Slc2a1.

Mouse T cells were stimulated and expanded for 10 days in vitro in the presence of 0.5 μM p38 inhibitor. Cellular and mitochondrial ROS levels were measured using FACS analysis. The results are shown in FIGS. 9B-9C. As shown in FIGS. 9B-9C, the total ROS and mitochondrial ROS were reduced following treatment with p38i.

Mouse T cells were stimulated and expanded for 5 days in vitro in the presence of 0.5 μM p38 inhibitor. SEAHORSE analysis was performed to determine spare respiratory capacity of cells. The results are shown in FIG. 9D. As shown in FIG. 9D, the total oxygen consumption increased following treatment with p38i.

### Example 5

This example demonstrates that inhibition of p38 MAPK improves the anti-tumor function of T cells for ACT.

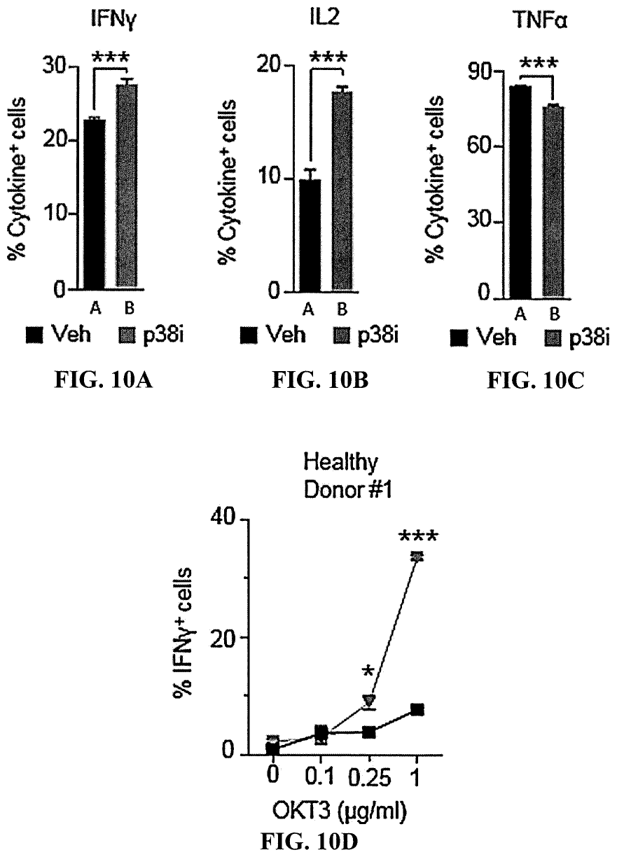

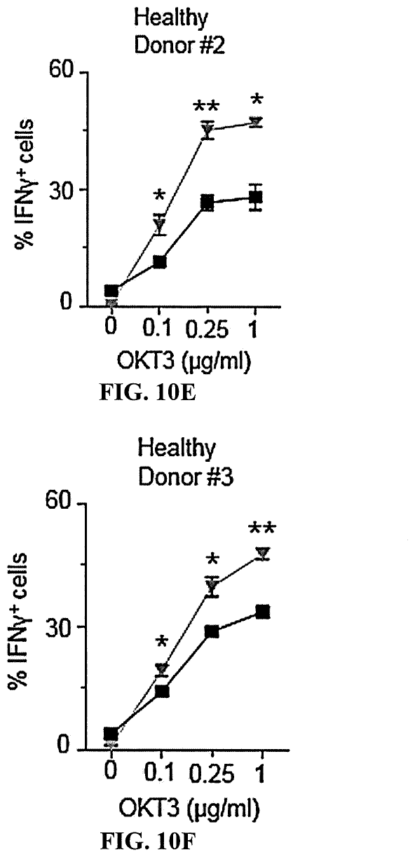

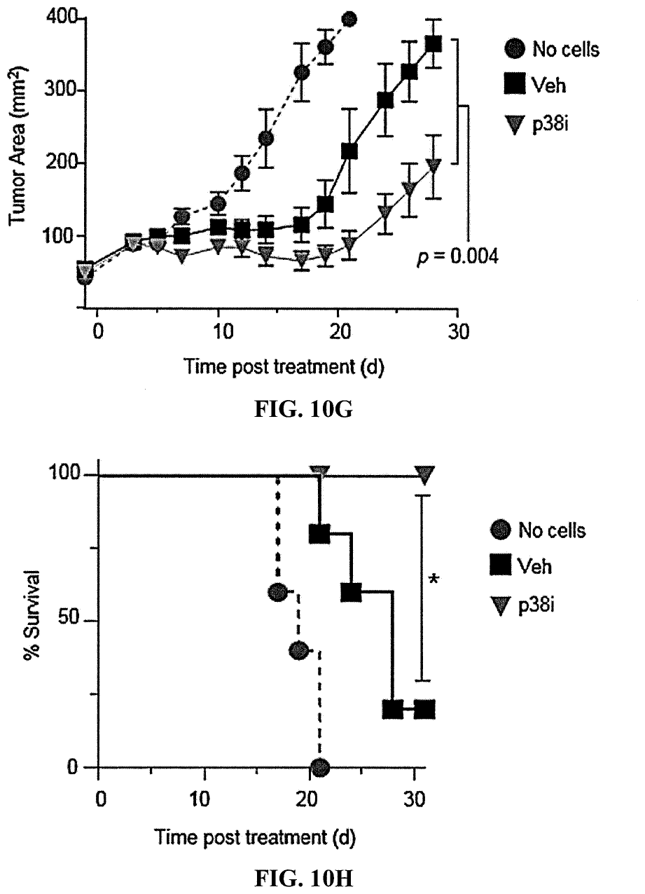

Mouse T cells were stimulated and expanded for 10 days in vitro in the presence of 0.5 μM p38 inhibitor. Cells were briefly stimulated with anti-CD3/anti-CD28 in the presence of GOLGI-STOP protein transport inhibitor and GOLGI-PLUG protein transport inhibitor to determine the cytokine production ability of cells. The results are shown in FIGS. 10A-10C. As shown in FIGS. 10A-10C, IFN-gamma and IL-2 production increased following p38i treatment. TNF alpha production decreased following p38i treatment.

Human T cells were expanded for 20-25 days in the presence of p38i. As shown in FIGS. 10D-10F, the p38i-treated cells possessed an enhanced ability to produce the effector cytokine IFN-gamma.

Subcutaneous tumor growth was measured in mice receiving adoptive cell transfer of Pmel-1 T cells which were expanded ex vivo for 10 days in the presence of p38 inhibitor. Tumor area (FIG. 10G) and overall survival (FIG. 10H) are shown. Significance for tumor growth kinetics were calculated by Wilcoxon rank-sum test. Survival significance was assessed by a log-rank Mantel-Cox test (n=5 mice per treatment group). As shown in FIGS. 10G-10H, p38i treatment enhanced the ability of the transferred cells to reduce tumor area and promote survival.

### Example 6

This example demonstrates that inhibition of p38 improves the anti-tumor function of human T cells transduced with a CAR for adoptive cell transfer.

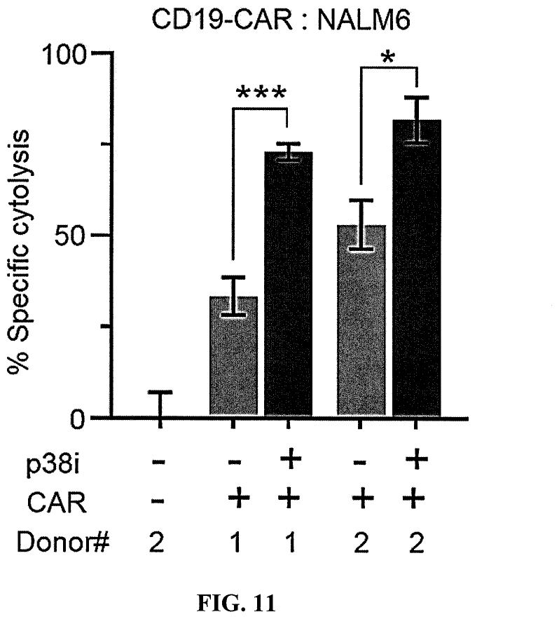

Human T cells from 2 healthy donors were transduced with a retrovirus encoding an anti-CD19 CAR and the numbers of cells were expanded for 20-25 days in the presence of p38i. Specific cytolysis was measured. The results are shown in FIG. 11. As shown in FIG. 11, CD19 antigen expressing NALM6 cells were lysed in higher numbers by T cells cultured in p38i condition. T cells and NALM6 cells were cocultured overnight.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing an isolated population of T cells for adoptive cell therapy, the method comprising culturing isolated T cells having antigenic specificity for a cancer antigen in vitro in the presence of a p38 mitogen activated protein kinase (p38 MAPK) inhibitor.

2. The method according to claim 2, further comprising introducing a nucleic acid encoding a chimeric antigen receptor (CAR) into the T cells in the presence of a p38 MAPK inhibitor and under conditions to express the CAR by the T cells.

3. The method according to claim 1, further comprising introducing a nucleic acid encoding an exogenous T cell receptor (TCR) into the T cells in the presence of a p38 MAPK inhibitor and under conditions to express the TCR by the T cells.

4. The method according to claim 1, wherein the T cells are tumor infiltrating lymphocytes (TIL).

5. The method according to claim 1, wherein the p38 MAPK inhibitor is a pharmacological p38 MAPK inhibitor.

6. The method according to claim 5, wherein the pharmacological p38 MAPK inhibitor is BIRB 796 (doramapimod), SB203580, SB202190 (FHPI), SB 239063, or LY2228820.

7. The method of claim 1, wherein the p38 MAPK inhibitor is an agent that inhibits expression of one or both of p38 MAPK mRNA and p38 MAPK protein.

8. The method of claim 7, wherein the p38 MAPK inhibitor is:
(i) an RNA interference (RNAi) agent,
(ii) a CRISPR-Cas system,
(iii) a zinc finger nuclease,
(iv) a transcription activator-like effector nucleases (TALEN) nuclease, or
(v) agent(s) which epigenetically inhibit expression of p38 MAPK.

9. The method according to claim 1, further comprising culturing the T cells in the presence of interleukin-2 (IL-2), interleukin-7 (IL-7), interleukin-15 (IL-15), interleukin-12 (IL-12) or a combination of two or more of the foregoing.

10. The method according to claim 1, wherein the population of T cells produced according to the method are CD8⁺.

11. The method according to claim 1, wherein the population of T cells produced according to the method are CD62L⁺.

12. The method according to claim 1, wherein the population of T cells produced according to the method provides an increase in any one or more of:
(i) persistence upon in vivo transfer;
(ii) proliferation upon in vivo transfer;
(iii) trafficking to tumor site(s) upon in vivo transfer;
(iv) antitumor activity upon in vivo transfer;
(v) catalase expression;
(vi) Slc2a1 expression; and
(vii) proportion of T cells with a central memory T cell (T_{CM}) phenotype
as compared to control T cells, wherein the control T cells are identical to the T cells that are cultured in the presence of a p38 MAPK inhibitor except that the control cells have not been cultured in the presence of a p38 MAPK inhibitor.

13. The method according to claim 1, comprising expanding the number of T cells in the presence of one or more non-specific T cell stimuli, one or more cytokines, and the p38 MAPK inhibitor.

14. The method according to claim 13, wherein expanding the number of T cells in the presence of the p38 MAPK inhibitor comprises culturing the cells for approximately 14 days in the presence of the p38 MAPK inhibitor.

15. The method according to claim 13, wherein the non-specific T cell stimuli are one or more of irradiated allogeneic feeder cells, irradiated autologous feeder cells, anti-CD3 antibodies, anti-4-1BB antibodies, and anti-CD28 antibodies.

16. The method according to claim 13, wherein the cytokine is IL-2.

17. An isolated population of T cells produced by the method according to claim 1.

18. A pharmaceutical composition comprising the isolated population of T cells of claim 17 and a pharmaceutically acceptable carrier.

19. A method for the treatment or prevention of cancer in a mammal, the method comprising administering to the mammal one or more T cells from the isolated population of T cells of claim 17 in an amount effective to treat or prevent cancer in the mammal.

20. The method claim 19, wherein the T cells are autologous to the mammal.
